(19) United States
(12) Patent Application Publication
(10) Pub. No.: US 2022/0160265 A1
(43) Pub. Date: May 26, 2022

(54) MACHINE-LEARNING BASED BIOSENSOR SYSTEM

(71) Applicants: Devangsingh Gajendarsingh Sankhala, Richardson, TX (US); Sriram Muthukumar, Allen, TX (US)

(72) Inventors: Devangsingh Gajendarsingh Sankhala, Richardson, TX (US); Sriram Muthukumar, Allen, TX (US)

(21) Appl. No.: 17/535,537

(22) Filed: Nov. 24, 2021

Related U.S. Application Data
(60) Provisional application No. 63/117,959, filed on Nov. 24, 2020.

(51) Int. Cl. A61B 5/1477; A61B 5/00; A61B 5/145; G16H 50/20

(52) U.S. Cl. CPC A61B 5/1477; A61B 5/7264; A61B 5/4266; G16H 10/40; A61B 5/4277; A61B 5/14507; G16H 50/20; A61B 5/14532

(57) ABSTRACT

Electrical characteristics of an electrical signal generated by an affinity-based senor are detected, where the affinity-based sensor is configured to bind to a particular biomarker within a body fluid sample and generate the electrical signal based on binding to the particular biomarker. One or more biometric characteristics of a subject are further detected from one or more other sensors. A data set comprising data describing each of the electrical characteristics and each of the one or more biometric characteristics is provided as an input to a machine learning model, which generates an output based on the input that identifies an amount of the particular biomarker present in the body fluid sample based on the input.

### CROSS-REFERENCE TO RELATED APPLICATION

This application claims priority to U.S. Provisional Application 63/117,959 filed on Nov. 24, 2020, the content of which is incorporated herein in its entirety.

### BACKGROUND

Early detection and reliable diagnosis can play a central role in making effective therapeutic decisions for treatment of diseases or managing certain physiological conditions. Detection may involve identification of disease-specific biomarkers in human body fluids that indicate irregularities in cellular regulatory functions, pathological responses, or intervention to therapeutic drugs.

Immunoassays can provide rapid and cost-effective mechanisms for detecting the presence and concentrations of analytes in a sample. Oftentimes, a single analyte (e.g. biomarker) or molecule may not be sufficient for unambiguous identification of specific diseases or for treating complex pathology conditions. In many cases, it is desirable to simultaneously detect the presence and concentration of more than one analyte in a sample, for example a variety of different analytes. More sensitive methods and devices for performing such tests are needed, that can enable users to perform quantitative measurements with higher accuracy and wider dynamic range than currently available biosensing devices.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1A is a simplified block diagram illustrating a biosensing system using electron-ionic mechanisms at fluid-sensor interfaces;

FIG. 1B is a simplified block diagram illustrating example details of embodiments of the biosensing system;

FIG. 1C is a simplified block diagram illustrating example operations and other example details of an embodiment of the biosensing system;

FIG. 2 is a simplified block diagram illustrating other example details of embodiments of the biosensing system;

FIG. 3 is a simplified block diagram illustrating yet other example details of embodiments of the biosensing system;

FIG. 4 is a simplified block diagram illustrating yet other example details of embodiments of the biosensing system;

FIG. 5 is a simplified block diagram illustrating yet other example details of embodiments of the biosensing system;

FIG. 6A is a simplified block diagram illustrating yet other example details of embodiments of the biosensing system;

FIG. 6B is a simplified block diagram illustrating yet other example details of embodiments of the biosensing system;

FIG. 6C is a simplified block diagram illustrating yet other example details of embodiments of the biosensing system;

FIG. 7 is a simplified block diagram illustrating yet other example details of embodiments of the biosensing system;

FIG. 8 is a simplified block diagram illustrating yet other example details of embodiments of the biosensing system;

FIG. 9 is a simplified circuit diagram illustrating yet other example details of embodiments of the biosensing system;

FIG. 10 is a simplified circuit diagram illustrating yet other example details of embodiments of the biosensing system;

FIG. 11 is a simplified diagram illustrating yet other example details of embodiments of the biosensing system;

FIG. 12 is a simplified circuit diagram illustrating yet other example details of embodiments of the biosensing system;

FIG. 13 is a simplified block diagram illustrating yet other example details of embodiments of the biosensing system;

FIG. 14 is a simplified block diagram illustrating yet other example details of embodiments of the biosensing system;

FIG. 15 is a simplified flow diagram illustrating example operations that may be associated with an embodiment of the biosensing system;

FIG. 16 shows a schematic of a sensing device in accordance with some embodiments;

FIG. 17 shows a sensing array comprising a plurality of sensing devices for detecting different target analytes;

FIG. 18 shows a multi-configurable sensing array comprising a plurality of sensing devices configured for simultaneous and multiplexed detection of a plurality of target analytes;

FIG. 19 shows a multi-configurable sensing array in accordance with some embodiments;

FIG. 20 shows a multi-configurable sensing system in accordance with some embodiments;

FIGS. 21A-21C show an SEM micrograph and ATR-FTIR spectra of ZnO nanostructures selectively grown on a working electrode, in accordance with some embodiments;

FIGS. 22A-22D show the functionalization of a working electrode in accordance with some embodiments;

FIGS. 23A-23D show fluid sample absorption onto different working electrodes and z-plane fragmentation using a modified EIS technique;

FIGS. 24A-24D show electrical simulation results for the sensing array of FIG. 20;

FIG. 25 shows a sensing platform comprising a test strip and a diagnostic reader device, in accordance with some embodiments;

FIG. 26 shows a sensing platform comprising a wearable device in accordance with some embodiments;

FIG. 27 is a flowchart showing a method for continuous, real-time detection of alcohol, EtG, and EtS in accordance with some embodiments.

FIGS. 28A-28F show different electrical field simulations for a multi-configurable sensing array comprising a plurality of electrodes; and

FIGS. 29A-29C show a modular sensing device in accordance with some embodiments; and

FIGS. 30A and 30B show a multi-configurable modular sensing array in accordance with some embodiments;

FIG. 31 is a diagram illustrating example training of a machine learning model;

FIGS. 32A-32C show diagrams illustrating example uses of machine learning models for use in determining an amount of a sensed biomarker;

FIGS. 33A-33C show diagrams illustrating example uses of machine learning models for use in determining an amount of a sensed biomarker;

FIG. 34 is a simplified block diagram showing a process for developing and training a machine learning model for use in determining an amount of a sensed biomarker;

FIGS. 35A-35C show an example implementation of a system utilizing a wearable sensor device to detect various biomarkers in sweat of a user;

FIG. 36 is a diagram illustrating use and evaluation of an example wearable sensor device;

FIG. 37 is a simplified block diagram illustrating an example system including a sensor device and coordinating personal computing device;

FIG. 38 is a block diagram illustrating an example use of the system of FIG. 37; and

FIG. 39 is a simplified flow diagram illustrating example processing of sensor data generated by an example sensor device.

### DETAILED DESCRIPTION

Reference will now be made in detail to exemplary embodiments of the disclosure, examples of which are illustrated in the accompanying drawings. Wherever possible, the same reference numbers will be used throughout the drawings and disclosure to refer to the same or like parts.

Provided herein are sensing devices, arrays of devices, and methods of using the same. Also provided herein are systems and devices configured to receive and analyze signals from the sensing devices or arrays, and provide an output based on the sensing results. Further provided herein are kits comprising modular sensing devices and arrays.

An example biosensor that facilitates biosensing using electron-ionic mechanisms at fluid-sensor interfaces is provided and includes a semiconductor sensing element, a first electrode and a second electrode located on a first plane of the sensing element with a first electric field being applied thereacross, a third electrode located on a second plane of the sensing element parallel to and removed from the first plane with a second electric field being applied across the first electrode and the third electrode perpendicular to the first electric field, and a dielectric substrate having a first portion that constrains a fluid including an analyte on a surface of the sensing element, and a second portion that facilitates dielectric separation of the fluid from the electrodes. The mutually perpendicular electric fields facilitate adjusting (e.g., tuning changing, modifying, etc.) a height of an electrical double layer in the fluid enabling detection and characterization of the analyte.

As used herein, the term “biosensor” can refer to any suitable sensor used in biochemical testing, biological testing, electrochemical testing, etc. The term “analyte” refers to a substance being identified, tested, characterized and otherwise measured; the analyte can comprise molecules of a single target species (e.g., glucose), or molecules of multiple target species (e.g., glucose and synthetic deoxyribose nucleic acid (DNA)). Examples of analyte include latex beads, lipid vesicles, whole chromosomes, cells and biomolecules including proteins and nucleic acids, gaseous molecules (e.g., ethylene), metal or semiconductor colloids and clusters, small molecules in the size range of sub-nanometer to millimeter, metabolites, and other such chemical molecules.

The various embodiments described herein may be useful for performing immunoassay tests on a sample, for example, to diagnose a disease or to provide information regarding a biological state or condition of a subject. The disclosed devices, arrays, systems, methods, and kits may be useful for detecting the presence and concentration of a wide variety of analytes in a sample. In many cases, the disclosed embodiments can enable simultaneous and multiplexed detection of the presence and concentration of multiple analytes in a single sample, via a common sensing platform. The various embodiments described herein are capable of detecting the presence and concentration of more than one analyte in a sample with greater specificity and/or sensitivity than currently available sensing devices or immunoassays. In many cases, the devices, arrays, systems, methods, and kits provided herein can enable a user to perform quantitative measurements with higher accuracy and wider dynamic range than currently available sensing devices or immunoassays.

As used in the specification and claims, the singular form “a”, “an” and “the” include plural references unless the context clearly dictates otherwise. For example, the term “a cell” includes a plurality of cells, including mixtures thereof.

As used in the specification and claims, the term “apparatus” may include a device, an array of devices, a system, and any embodiments of the sensing applications described herein.

As used herein, the term “about” a number refers to that number plus or minus 10% of that number. The term “about” a range refers to that range minus 10% of its lowest value and plus 10% of its greatest value.

Presently, there is a need for multiplexed immunoassays that can be used for simultaneous detection of multiple analytes in a short period of time, from a small sample volume, and at reduced costs. A key challenge lies in quantitative detection of biomarkers in a simultaneous or multiplexed manner at the early stages of a disease, especially if the sample contains very low concentrations of the biomarkers. To address this challenge, accuracy in diagnosis of the disease can be enhanced by quantification through a panel of biomarkers indicative or associated with the disease. Accordingly, there is interest and value in designing ultrasensitive sensing devices that are capable of detection of a panel of biomarkers from a single sample of human body fluids.

A number of transduction mechanisms can be used to achieve ultra-sensitive and multiplexed label-free biomarker detection. An example of such transduction mechanisms may include electrical/electrochemical-based sensing platforms, which typically involve capturing biomarkers on the surface of electrode materials. This phenomenon transduces the biological signal into a measurable electrical signal response, which can then be used to detect the presence and concentration of the biomarker in the sample. The structural and morphological characteristics of the electrode materials play an important role in achieving both sensitivity and selectivity required for ultrasensitive biomarker detection. Precise control over size and shape of the materials on a nanoscale level can yield nanostructures with enhanced chemical and physical properties, that can be tailored towards the design of robust ultrasensitive sensing platforms. For example, the availability of a large number of surface atoms in extended (out-of-plane) nanostructures can allow amplification of a biological signal response, when compared to their planar sensing electrode counterparts, thereby enabling improved sensing characteristics.

Detection of analytes can be based upon enzymatic sensing devices for the detection of glucose, cholesterol, lactic acid, uric acid, etc. Quantification of such analytes may be based upon detection of byproducts of enzymatic reactions where non-specific interactions may be an issue. Technological bottlenecks associated with non-specific interactions can be minimized by use of specific capture probes. For example, affinity-based sensing mechanisms for designing immunoassay-based sensing devices using non-faradic approaches can be used. In some cases, semiconducting nanostructures can be used to facilitate direct electron transport as their electrical properties are strongly altered by charge perturbations occurring due to biomolecular confinement and binding events. The electrical detection/sensing methods described herein can permit direct characterization of capture probe—target biomarker interaction, based on charge perturbations at the electrode/electrolyte interface.

When an electrode comprising nanostructures on its surface is exposed to an ionic solution containing biomolecules, a potential difference can be created at the electrode/electrolyte interface due to unequal distribution of charges. As a result of biomolecular binding events at the nanostructured electrode surface, redistribution of charges in the electrode and ions in the electrolyte can result in formation of a space-charge region within the nanostructures and at an electrical double layer at the electrode/electrolyte interface. Biomarker binding can be evaluated and quantified by measuring changes in electrode impedance and/or capacitance at selected frequencies. In some embodiments, changes to the space-charge capacitance and overall impedance at the electrode/electrolyte interface can be measured using both Mott-Schottky technique and a modified electrochemical impedance spectroscopy (EIS) technique which are described in detail herein. A correlation in output signal response with concentration can be determined between (and using) both detection techniques, which provide a combinatorial approach for the accurate and sensitive detection of protein biomarkers.

The electrochemical sensing devices, arrays and methods described herein can be used for detecting multiple biomarkers. The sensing devices and arrays can be designed and fabricated on various substrates. The substrates may be rigid or flexible. Examples of suitable substrates may include silicon, glass, printed circuit boards, polyurethane, polycarbonate, polyamide, polyimide, and the like. The sensing devices and arrays can be used for continuous and real-time detection, monitoring, and quantification of various chemical and biological agents in body fluids. Examples of body fluids may include blood, sweat, tears, urine, saliva, and the like. Real-time detection can be performed in a single-use or in a continuous-use manner using the sensing technology platform described herein. The challenges of multiplexed detection of specific proteins can be addressed by the present inventions, which are directed to: (1) the designs of a microelectrode sensor platform comprising an array of multi-configurable sensing device each independently functionalized for specific detection of a target biomarker(s), and (2) each sensor output/results being independently measured and transduced to provide a combinatorial outcome relating to the end physiological state being predicted.

An important aspect in affinity-based sensing devices relates to the specificity of the sensor. The term “specificity” may be described as the ability of the sensor to respond specifically to target biomolecules, but not to other similar biomolecules. Generally, current electrical-based label-free sensing devices are often unable to distinguish between specific and nonspecific interactions except via probe specificity, regardless of the readout method. Specificity is often important for detection of biomolecules in real-world samples such as blood, serum, urine, saliva, sweat, etc., where the target concentration can be much lower than the concentration of non-target biomolecules present in the samples. For instance, blood serum typically contains around 70 mg/mL total protein content; however, disease biomarker proteins may be expressed in concentrations in the lower pg/mL regime. Thus, a sensing device that can detect 1 pg/mL of the protein in a saline solution but manifests a 1 ng/mL response in blood, may not be useful in a clinical setting unless the serum is depleted of interfering plasma proteins, or if some other compensations were made.

In the various embodiments described herein, specificity to the detection of target biomarkers, within each sensor on the platform array, can be achieved through specific antibody immobilization on microelectrode surfaces having semiconducting nanostructures (e.g. ZnO), functionalized using thiol-based and/or phosphonic-based linker chemistries to achieve stable and robust immobilization of the proteins. Target protein specific monoclonal antibodies can be introduced onto the linker functionalized nanostructured ZnO surfaces in the presence of a room temperature ionic liquid (RTIL) electrolyte buffer. The properties of the RTIL can be adjusted to ensure long term stability (prevent denaturing of the protein antibody from pH, temperature and environment), and enhance the efficacy in selective binding to the nanostructured ZnO surfaces. A modified electrochemical impedance spectroscopy (EIS) technique as described herein can be used for enabling ultra-sensitive and highly-specific detection of proteins.

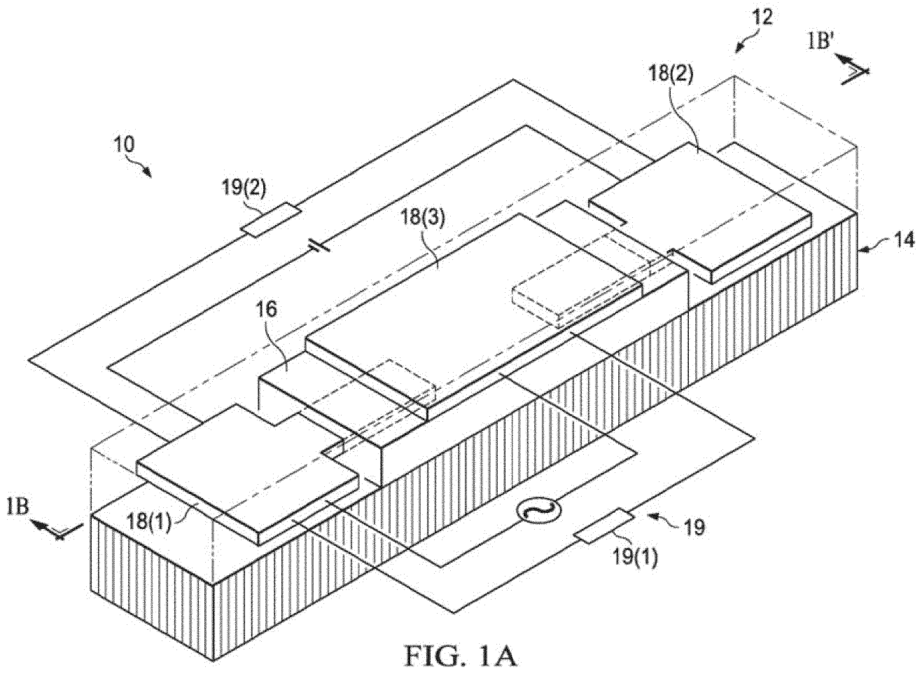

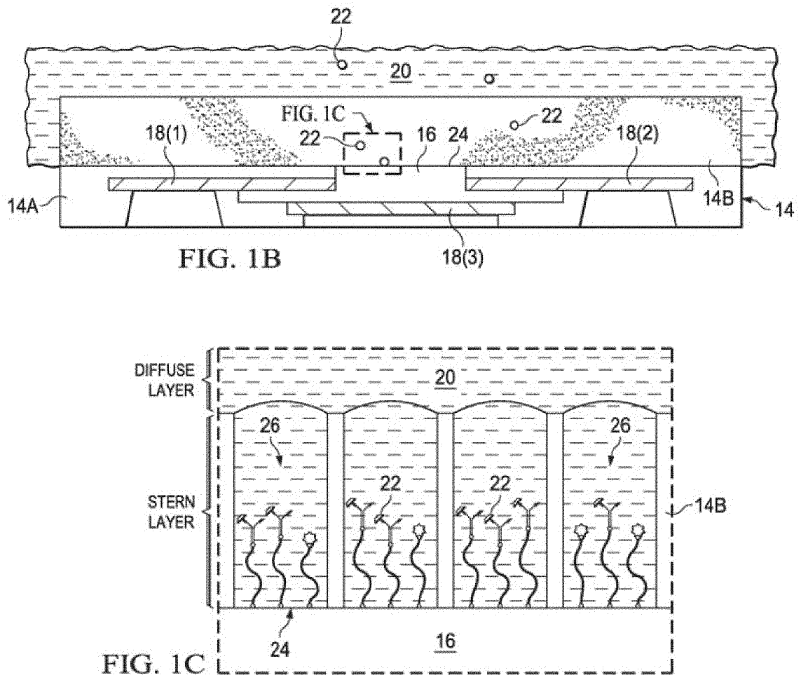

Turning to FIGS. 1A-1C, FIGS. 1A-1C are simplified block diagrams illustrating a biosensing system 10 for facilitating biosensing using electron-ionic mechanisms at fluid-sensor interfaces in accordance with one example embodiment; FIG. 1B is a cross-section along axis B-B′; and FIG. 1C is an example detail of the cross-section. FIG. 1A illustrates a biosensing system 10 comprising a biosensor 12 including a substrate 14, a sensing element 16, a plurality of electrodes 18(1)-18(3), and an output 19 comprised of two components, baseline 19(1) and response 19(2).

A transverse voltage may be applied across some of electrodes 18 (e.g., 18(1) and 18(2)); an orthogonal voltage may be applied across other electrodes 18 (e.g., 18(1) and 18(3)). Electrodes 18 (e.g., 18(1) and 18(2)) across which the transverse voltage is applied may be referred to as ‘transverse electrode;’ electrodes 18 (e.g., 18(1) and 18(3)) across which the orthogonal voltage is applied may be referred to as ‘orthogonal electrodes.’ In a general sense, ‘transverse’ and ‘orthogonal’ refer to direction of electric fields produced by the respective voltages; in various embodiments, the electric field produced by the transverse voltage is perpendicular to the electric field produced by the orthogonal voltage. In some embodiments, the transverse voltage may comprise direct current (DC) voltage and the orthogonal voltage may comprise alternating current (AC) voltage. In other embodiments, the transverse voltage may comprise AC voltage, and the orthogonal voltage may comprise DC voltage. In yet other embodiments, the transverse voltage may initially comprise AC voltage, which may be switched to DC voltage, and the orthogonal voltage may comprise AC voltage.

Baseline 19(1) comprises impedance, or capacitance, or current measured across orthogonal electrodes 18(1) and 18(3) and establishes a baseline value for the respective measurement; response 19(2) comprises impedance, or capacitance, or current measured across transverse electrodes 18(1) and 18(2). In various embodiments, comparison between baseline 19(1) and response 19(2) can indicate a signal-to-noise ratio (SNR) of the measurements and provide detection and/or measurement of concentration of an analyte 22 in a fluid 20.

Substrate 14 generally allows for fluid containment such that a portion of fluid 20 comprising analyte 22 is in contact with sensing element 16 at a fluid-sensor interface 24, as indicated in FIG. 1B. Note that fluid containment is in three dimensions, for example, both vertically and laterally (e.g., perpendicular and parallel to sensing element surface.) Fluid-sensor interface 24 comprises a zone of interaction between sensing element 16 and fluid 20. In some embodiments, fluid-sensor interface 24 comprises a surface of sensing element 16 in contact with fluid 20; in other embodiments, fluid-sensor interface 24 comprises an additional layer of linker molecules that are bound to the surface of sensing element 16; in yet other embodiments, fluid-sensor interface 24 comprises an additional layer of capture probes that bind to the linker molecules. In yet other embodiments, fluid-sensor interface 24 additionally comprises a layer of fluid 20 including an electrical double layer (EDL).

In some embodiments, as indicated in FIG. 1B, substrate 14 may comprise two separate portions, indicated as 14A and 14B. In an example embodiment, portion 14A comprises a hydrophobic biocompatible material (e.g., Parylene™) and portion 14B comprises a porous biocompatible hydrophilic membrane (e.g., polyimide, polyamide, nylon, alumina, polycarbonate, polymer, ceramic, etc.). In various embodiments, portion 14A may prevent direct interaction between fluid 20 and electrodes 18(1)-18(3), for example, providing dielectric separation (e.g., electrical isolation) of electrodes 18(1)-18(3) from fluid 20. In some embodiments, portion 14B may provide a fluid containment zone allowing analyte 22 of fluid 20 to bind to sensing element 16 at fluid-sensor interface 24.

Some of electrodes 18(1)-18(3) (e.g., 18(1) and 18(2)) may be located on one plane, and the other electrodes (e.g., 18(3)) may be located on another, different plane. In an example embodiment, transverse electrodes 18(1) and 18(2) may be located on a first plane of sensing element 16 and orthogonal electrode 18(3) may be located on a second plane of sensing element 16 parallel to and removed from the first plane.

To explain the fluid containment in more detail, as indicated in FIG. 1C, portion 14B may comprise pores 26 that provide a fluid containment zone allowing analyte 22 to bind to sensing element 16 at fluid-sensor interface 24 in the presence of an electric field. In some embodiments, pores 26 may comprise nanopores (e.g., diameter or size in the order of nanometers). In various embodiments, the electric field produced by the orthogonal voltage causes reversible aggregation of analyte 22 in fluid 20 into planar aggregates adjacent to fluid-sensor interface 24. The planar aggregation disassembles when the electric field is removed. In confined geometries, as in pores 26, the surface charge distribution on sensing element 16 and topography of bounding electrodes 18(1) and 18(2) may determine a nature of electron-ion interaction at fluid-sensor interface 24. The planar aggregation can include organization similar to self-assembly producing partial coverage, monolayer coverage or stretched coverage.

In various embodiments, fluid 20 wicks through pores 26 to make contact with sensing element 16 at fluid-sensor interface 24. In a general sense, when sensing element 16 having surface charge is immersed in fluid 20 containing ions, a diffuse ion cloud, called the “stern layer” forms in fluid 20 to screen (e.g., neutralize) sensing element 16's surface charge. Beyond the stern layer is a diffuse layer comprising ions providing an electrical gradient within fluid 20. The arrangement of a layer of (immobile) charges in the stern layer and the screening cloud of (mobile) counter-ions in the diffuse layer of fluid 20 is referred to as the electrical double layer (EDL). As noted previously, fluid-sensor interface 24 comprises the EDL. In the EDL of small but finite thickness, fluid 20 is not electroneutral. Consequently, electric fields acting on the EDL will set in motion ions in the diffuse layer, and these will in turn entrain surrounding fluid 20. The resulting flow fields reflect the spatial distribution of ionic current in fluid 20.

The diffuse layer may be polarized by the orthogonal electric field (i.e., the electric field produced by the orthogonal voltage) to effect charge perturbation associated with detection of target species of analyte 22 in fluid 20. The effective ionic content of the combination of the stern layer and the diffuse layer acts as a screen (e.g., charge screening) preventing the target species of analyte 22 from travelling to and binding to sensing element 16. However, excluded volume effect (e.g., ‘excluded volume’ of a molecule is the volume that is inaccessible to other molecules in the system as a result of the presence of the molecule) and macromolecular crowding from non-specific target species in the confined spaces (e.g., pores 26) can minimize such charge screening. Embodiments of biosensing system 10 can facilitate multiple target species detection in varying fluids; analyte 22 may comprise target species with no charge, high charge or low charge and fluid 20 may have with varying polarity levels within the broad scope of the embodiments.

For purposes of illustrating the techniques of biosensing system 10, it is important to understand the communications that may be traversing the system shown in FIG. 1. The following foundational information may be viewed as a basis from which the present disclosure may be properly explained. Such information is offered earnestly for purposes of explanation only and, accordingly, should not be construed in any way to limit the broad scope of the present disclosure and its potential applications.

Various approaches to frequent and/or continuous biosensing tend to fall into two general categories: “non-invasive” and “minimally invasive.” Non-invasive monitoring determines analyte (e.g., a substance whose chemical constituents are being identified and measured) levels by directly tracking spectroscopic changes in skin and tissue. Infrared radiation and radio wave impedance spectroscopy are examples of this technology. Progress with these approaches has been slow for various reasons, such as need for frequent calibration, reproducible sample illumination, and variances in spectroscopic backgrounds between individuals. The “minimally invasive” approach avoids direct extraction of biological fluids from the body and relies on monitoring of signal changes in the biological fluids using an intermediate sensing element. Biosensors of this type typically provide specific quantitative or semi-quantitative analytical information using a biological recognition element in combination with a transducing (e.g., detecting) element.

In a general sense, typical modalities for biochemical sensing of minimally invasive biosensors utilize affinity reactions and binding as a means to transduce (e.g., convert, change, alter, etc.) the chemical sensing into optical, electrical, or mechanical signal or a combination thereof (the basic principle being predicated on binding between components of a reaction pair (e.g. antigen/antibody, hapten/antibody, etc.) where, in some cases, one component is labeled so as to be easily analyzed by some external means). Examples of specific binding substances that have been historically targeted using biosensors include antibodies, antigens, enzymes, enzyme substrates, enzyme substrate analogs, agglutinins, lectins, enzyme cofactors, enzyme inhibitors and hormones.

For example, typical biosensors utilize at least one of three different bio-sensing modalities: (1) electrical biosensors, (2) optical biosensors, and (3) mechanical biosensors. The input to such biosensors are biological molecules (e.g., molecules from biological sources, such as animals and plants). In electrical biosensors, the transduction (e.g., conversion or conveyance of energy in one form from a donor to another form at a receptor) is biochemical to electrical; in optical biosensors, the transduction is biochemical to optical to electrical; and in mechanical biosensors, the transduction is biochemical to mechanical to electrical. The measurable outputs in electrical biosensors include current, voltage and/or impedance; the outputs in optical biosensors include light intensity, and refractive index; the outputs in mechanical biosensors include resonance frequency and mass.

In an example biosensor that senses glucose concentration, an optical conduit, such as an optical fiber has an optical system at a proximal end of the optical conduit and a sensing element attached to a distal end. The sensing element includes a binding protein that binds with a target analyte, and a reporter group that undergoes a luminescence change with changing analyte concentrations. In another example, a graphene electrode is linked to a biosensing element, which is bonded to a flexible substrate. The graphene electrode has a positive terminal end and a negative terminal end; an electrical voltage is applied to the positive and negative terminals to measure an electrical current response in proportion to a lactate concentration on the biosensing element.

In yet another example, the biosensor includes a nanotube, with a lipid bilayer around the nanotube, and a sensing element connected to the lipid bilayer, the biosensor capable of detecting variations in ion transport through a protein pore. The biosensor further includes a gate electrode; a source electrode; and a drain electrode, with the nanotube connected to the gate electrode, the source electrode, and the drain electrode. Yet another example biosensor includes a selectively permeable interface membrane, a porous protein-receiving matrix adjacent to the interface membrane, an indicating electrode, an inlet conduit through which fresh protein conjugate may flow to the protein-receiving matrix, and an outlet conduit through which spent protein conjugate may be removed from the protein-receiving matrix. The selectively permeable interface membrane may be used to separate biochemical, optical or other processes from the analyte. The biosensor's in situ probe provides continuous, real-time analysis by amperometric detection of hydrogen peroxide produced as a by-product of enzymatic oxidation of a substrate by its enzyme catalyst at the probe.

In yet another biosensor, electrochemical sensors employ an ion selective electrode to detect a reaction product of an enzyme that acts as a label for one component of a specific binding pair. The biosensor includes electrically semiconductive material to which an analyte specific binding substance is suitably immobilized. By placing the analyte specific binding agent in close proximity to the semiconducting material, a change in an electrical field occurs as a result of the binding reaction, which in turn effects a change in the properties of the semiconducting material that can be measured suitably.

Such currently available sensor technologies primarily perform biometric assessments, which may not be sufficient for determining cohesive response strategies. Moreover, they typically require complex setup and trained personnel for operation and analysis. Challenges in such currently existing wearable technologies include amplifying signals using reporter molecules, use of redox probes, and low signal-to-noise ratio (SNR) without use of amplifiers. In typical biosensor technologies, it may be desirable to enable wearable and non-invasive sensor technologies that allow users to rapidly evaluate their physiological status in a continuous manner. For example, wearable, non-invasive sensors that monitor chemical and biological agents without requiring constant recalibration may be desired for maintaining stasis in humans and surrounding environments.

Biosensing system 10 is configured to address these issues (among others) to offer a system and method for facilitating biosensing using electron-ionic mechanisms at fluid-sensor interfaces. Embodiments of biosensing system 10 provide for charge transfer modulation and characterization of analyte 22 at fluid-sensor interface 24 with sensing element 16. In various embodiments substrate 14 may comprise any suitable insulating material, flexible, or rigid, that can effectively contain (e.g., constrain, enclose, hold, surround, channel, encompass, enfold, ring, etc.) fluid 20. Examples for suitable materials for substrate 14 include polymers, ceramics, glass, or combination thereof.

In one example embodiment, substrate 14 comprises a flexible polymer having nano-pores that facilitate contact of fluid 20 in the nano-pores with sensing element 16. In another example embodiment, substrate 14 may comprise a porous membrane that allows for fluid 20 to wick to sensing element 16 and provides support to biosensor 12. Substrate 14 may also include a hydrophobic material, such as Prylene™, that forms an isolation barrier between wicked fluid 20 in the membrane and electrodes 18(1)-18(3). Parylene™ is an example of a hydrophobic biocompatible material that can form a non-conducting isolation barrier such that the output of biosensor 12 captures electron-ion interaction at fluid-sensor interface 24 between fluid 20 and sensing element 16, and does not capture any direct interaction of fluid 20 with electrodes 18(1)-18(3).

In various embodiments, sensing element 16 provides for binding (e.g., attaching, tying, tethering, adhering, etc.) of analyte 22 at fluid-sensor interface 24 and charge transfer modulation therefrom. Sensing element 16 may comprise any suitable semiconducting (e.g., semi-insulating) material that allows for signal transduction and modulation between electrodes 18(1)-18(3) and analyte 22. In a general sense, the properties of the semiconducting material that provide for its semiconductive characteristics depend on a number of electrons in that material available to move freely through the material under the influence of an externally applied electric field. Any suitable semiconducting material appropriate to the assay protocol may be used within the broad scope of the embodiments. For example, a stack formed with ZnO thin films can be functionalized with selective linker chemistry (e.g., thiol, carboxylic, amine, etc.) to conjugate with (e.g., bind to) specific target species of analyte 22. The molecules facilitating the linker chemistry are referred to as capture probes; the capture probes can comprise proteins or small molecules (e.g., antibodies, nucleic acids, etc.) that can detect a specific target species of analyte 22; the capture probes may be immobilized on the surface of sensing element 16 at fluid-sensor interface 24.

An example material of semiconducting material used in sensing element 16 is zinc oxide (ZnO). Other examples include diamond (C), silicon (Si), germanium (Ge), tin (Sn), silicon carbide (SiC), Sulphur (S₈), boron nitride (BN), boron phosphide (BP), boron arsenide (BAs, B₁₂As₂), aluminum nitride (AlN), aluminum phosphide (AIP), aluminum arsenide (AlAs), aluminum antomonide (AlSb), gallium nitride (GaN), gallium phosphide (GaP), gallium arsenide (GaAs), gallium antimonide (GaSb), indium nitride (InN), indium phosphide (InP), indium arsenide (InAs), indium antimonide (InSb), cadmium selenide (CdSe), cadmium sulphide (CdS), cadmium telluride (CdTe), zinc selenide (ZnSe), zinc sulfide (ZnS), zinc telluride (ZnTe), cuprous chloride (CuCl), copper sulfide (Cu₂S), lead selenide (PbSe), lead sulfide (PbS), lead telluride (PbTe), tin sulfide (SnS), tin sulfide (SnS₂), tin telluride (SnTe), lead tin telluride (PbSnTe), thallium tin telluride (Tl₂SnTe₅), thallium germanium telluride (Tl₂GeTe₅), bismuth telluride (Bi₂Te₃), cadmium phosphide (Cd₃P₂), cadmium arsenide (Cd₃As₂), cadmium antimonide (Cd₃Sb₂), zinc phosphide (Zn₃P₂), zinc arsenide (Zn₃As₂), zinc antimonide (Zn₃Sb₂), titanium dioxide (TiO₂), cuprous oxide (Cu₂O), cupric oxide (CuO), uranium dioxide (UO₂), uranium trioxide (UO₃), bismuth trioxide (Bi₂O₃), tin dioxide (SnO₂), barium titanate (BaTiO₃), strontium titanate (SrTiO₃), lithium niobate (LiNbO₃), lanthanum copper oxide (La₂CuO₄), lead iodide (PbI₂), molybdenum disulfide (MoS₂), gallium selenide (GaSe), tin sulfide (SnS), bismuth sulfide (Bi₂S₃), gallium manganese arsenide (GaMnAs), indium manganese arsenide (InMnAs), cadmium manganese telluride (CdMnTe), lead manganese telluride (PbMnTe), lanthanum calcium manganite (La_{0.7}Ca_{0.3}MnO₃), ferric oxide (FeO), nickel oxide (NiO), chromium bromide (CrBr₃), copper zinc tin sulfide (CZTS), tungsten sulfide (WS₂), tungsten selenide (WSe₂), vanadium dioxide (VO₂), graphene oxide, etc.

In various embodiments, electrodes 18(1)-18(3) may comprise any suitable conducting material, such as copper or gold, that does not react with fluid 20. In various embodiments, electrodes 18(1)-18(3) form Ohmic (e.g., resistive) electrical contact with sensing element 16. In various embodiments, fluid 20 may comprise any suitable fluid including liquids, gels, colloids, gases and/or combination thereof. Examples of fluid 20 include body fluids, such as sweat, blood, tears, serum, saliva, urine, etc.; and non-body fluids such as vapors (from fruits, milk, and other foods), aqueous and non-aqueous solutions, etc. Analyte 22 may correspond to various biomolecules being tested, such as glucose, lactose, ethylene, urea, salt (NaCl), etc.

Binding of analyte 22 at fluid-sensor interface 24 may occur through electro-chemical, electro-ionic, polarization, and other charge-based modes that causes work function tuning of the semiconducting material of sensing element 16, resulting in modulation of space-charge capacitance and electrical double layer capacitance. (Note that work function of a semiconductor material is a property of a surface of the material, and corresponds to a minimum energy required to remove an electron from an interior of the material to a point immediately above the surface of the material; the term “immediately” referencing a distance that is large in atomic scale, but small in terms of electrical fields). In a general sense, semiconductor interfaces, such as fluid-sensor interface 24 in fluid 20 comprising ions, experience disparate electrochemical potential at fluid-sensor interface 24.

At equilibrium, an exchange of charges occurs between sensing element 16 and fluid 20 resulting in charge redistribution at fluid-sensor interface 24. The localized charge redistribution in sensing element 16 is referred to as space-charge capacitance (C_{sc}); the localized charge redistribution in fluid 20 comprises the electrical double layer (EDL) capacitance (C_{edl}). The space-charge capacitance is typically a function of the semiconductor material of sensing element 16; different semiconductor materials exhibit different space-charge capacitances to the same electric field. Hence a measured total capacitance across fluid-sensor interface 24 at equilibrium derives from the material-specific space-charge capacitance and a capacitive impedance associated with molecules in the EDL binding to fluid-sensor interface 24.

Typically, EDL capacitance of fluid 20 may be negligible compared to the space-charge capacitance (i.e. C_{edl}>>C_{sc}) of sensing element 16. However, where biochemical binding events occur at fluid-sensor interface 24 in confined spaces due to fluid containment by portion 14B of substrate 14, the EDL capacitance can be significant and matched in magnitude to the space-charge capacitance (i.e. C_{edl}≈C_{sc}). In some embodiments, for example, where the EDL capacitance is matched in magnitude to the space-charge capacitance, changes to the EDL capacitance with varying concentrations of analyte 22 may be proportionally reflected in similar changes to the space-charge capacitance. Confinement of fluid 20 to an active area of biosensor 12 may enhance the charge transfer between sensing element 16 and fluid 20 and consequent effects. The confinement may be achieved through suitably sized pores 26 in substrate 14 (e.g., using a porous membrane, such as in portion 14B).

Further, the total capacitance across fluid-sensor interface 24 may vary with the binding interactions at fluid-sensor interface 24; the binding interactions may vary with the specific molecule binding to sensing element 16. Tunable electron-ionic mechanisms resulting from the biochemical binding events within the confined spaces at fluid-sensor interface 24 may be measured and/or characterized using electrical parameters, such as current, voltage, impedance and capacitance. In some embodiments, input voltages are applied; in other embodiments, current sources are used to generate desired voltages across electrodes 18(1)-18(3); in yet other embodiments, a steady state potential of different amounts is maintained across electrodes 18(1)-18(3). Output 19 from biosensor 12 may include impedance in some embodiments; current in other embodiments; and capacitance in yet other embodiments. Some embodiments of biosensing system 10 can tune biosensor 12 to distinguish between capacitance changes from biochemical analyte binding and from space charge modulation.

In various embodiments, fluid-sensor interface 24 comprises a portion of the EDL. In some embodiments, the sensitivity of biosensor 12 may vary with the EDL thickness; a particular EDL thickness may be conducive to detect a corresponding target species of analyte 22. The height of fluid-sensor interface 24 may be indicative of a volume of fluid 20 above the surface of sensing element 24 and can correlate with the sensitivity of biosensor 12; for example, height h₁ of fluid-sensor interface 24 may correspond to high sensitivity detection of glucose, but low sensitivity detection of cortisol; height h₂ of fluid-sensor interface 24 may correspond to high sensitive detection of cortisol, but low sensitivity detection of glucose; etc.

In some embodiments, electrokinetic focusing using polarization principles may be used to achieve particle separation (e.g., screening) in fluid 20, which may further enhance EDL capacitance modulation and/or sensitivity of biosensor 12. In a general sense, the EDL varies based on the presence or absence of specific target biomolecules in fluid 20. Charge modulation in the EDL may be further controlled by applying an orthogonal electric field (with respect to transverse electrodes 18(1) and 18(2)) to sensing element, forming an electrically modulated gate. In some embodiments, the transverse electric field provides a bias voltage (e.g., around which the response of biosensor 12 may be linear, gain may be high, etc.) and the orthogonal electric field provides a measure of the capacitance of the EDL. The orthogonal electric field can also enable pinning of the EDL and tuning a height of an electro-ionic interface height (e.g., height of fluid-sensor interface 24 including the EDL), facilitating segmenting the EDL capacitance and the space-charge capacitance enabling higher sensitivity of biosensor 12.

Because the capacitance is influenced by frequency of the electric field, AC voltage of varying amplitude and frequency may be applied to orthogonal electrodes 18(1) and 18(3) to cause changes to capacitance that can be measured with higher sensitivity. Embodiments of biosensing system 10 can facilitate separately detecting and characterizing multiple different target species of analyte 22 using the same biosensor 12, for example, by varying the orthogonal electric field with respect to the transverse electric field. The orthogonal electric field, generated between orthogonal electrodes 18(1) and 18(3) may facilitate differentiation of target species of analyte 22 from each other; for example, orthogonal electric field E₁ generated using AC voltage of amplitude V₁ and frequency f₁ may cause target species S₁ to migrate to fluid-sensor interface 24; another orthogonal electric field E₂ generating using AC voltage of amplitude V₂ and frequency f₂ may cause target species S₂ to migrate to fluid-sensor interface 24; and so on.

In some embodiments, transverse electrodes 18(1) and 18(2) are fabricated on porous membranes comprising portion 14B of substrate 14 and passivated for electrical isolation with a dielectric comprising portion 14A of substrate 14. Semiconductor material, such as ZnO, graphene, MoS₂, VO₂, etc. of sensing element 16 is deposited across transverse electrodes 18(1) and 18(2) to provide a desired surface area for biochemical binding events. Orthogonal electrode 18(3) is deposited on a surface of sensing element 16, distal from the surface where the binding reactions occur, for example, to electrically modulate the surface charge distribution and gate the flow of charges across transverse electrodes 18(1) and 18(2).

Embodiments of biosensing system 10 can include nanoporous and/or nanostructure materials for performing real-time detection of analyte 22 in fluid 20. The nanoporous and/or nanostructure material may allow wicking of fluid 20 and also enhance detection of specific biochemical binding events on the semiconducting material surface of sensing element 16 without charge screening from non-specific constituents in fluid 20. In some embodiments, a Debye length (e.g., a measure of a charge carrier's net electrostatic effect in solution, comprising a length along which the electrostatic effects persist; the Debye length is generally a radius of a sphere outside of which charges are screened) formed at fluid-sensor interface 24 may be maximized; the Debye length may be measured and quantified as the EDL (e.g., the Debye length is indicative of a thickness of the EDL). Debye length measurement and tuning may be performed with orthogonal electrodes 18(1) and 18(3). In various embodiments, baseline 19(1) may be indicative of a baseline value for the Debye length. The transverse and orthogonal electric fields, which are mutually perpendicular to each other, may facilitate detecting and characterizing multiple target species irrespective of their charge status. Further, the orthogonal electric field may be tuned to manipulate and/or measure various Debye lengths of the EDL in fluid 20, facilitating isolation of different target species according to the Debye length.

In some embodiments, EDL probing may facilitate understanding of molecular information in fluid 20, and may be suitable for ultra-low power electronics. In various embodiments, the charge screening effects may be characterized by various models (e.g., theories), including Helmholtz model, Gouy-Chapman model, and Gouy-Chapman-Stern model. For example, according to the Gouy-Chapman-Stern model, the EDL may be characterized as a capacitance in an electrical circuit, the capacitance varying according to predetermined functions (e.g., relationships, formulae) of material properties and molecular constituents of fluid 20.

In various embodiments, biosensor 12 can operate in ultra-low power modes, and can detect and/or diagnose concentration of analyte 22 for various modes of operation, including: single-species (e.g., analyte 22 comprises a single target species of interest), single input/output (I/O) mode, single-use (e.g., biosensor 12 discarded after single use); single-species, multi-I/O mode, single-use; multi-species (e.g., analyte 22 comprises more than one target species of interest), single-I/O mode, single-use; multi-species, multi-I/O mode, single-use; single-species, single-I/O mode, multi-use (e.g., biosensor 12 reusable for multiple tests); single-species, multi-I/O mode, multi-use; multi-species, single-I/O mode, multi-use; multi-species, multi-I/O mode, multi-use.

In some embodiments, analog-to-digital signal conversion may be employed for sensing and processing output 19 (e.g., baseline 19(1) and response 19(2)) from sensing element 16. In other embodiments, digital to analog signal conversion may be employed for sensing and processing. In yet other embodiments, mixed signal circuits may be employed for sensing and processing. Data communication with an end user may be included in biosensing system 10, for example, to convert analog or digital signals to meaningful user information (e.g., impedance changes in sensing element 16 converted into digital signals, which in turn are converted into a readout of target species concentration level on a display).

In some embodiments, output 19 may be communicated (e.g., through wired or wireless mechanisms) to smart portable devices, and other computing devices, for example, for further analysis. In other embodiments, output 19 may be communicated (e.g., through wired or wireless mechanisms) to light based display devices (e.g., light emitting diode (LED) display, OLED etc. based status displays)

Embodiments of biosensing system 10 may be used to detect and/or analyze presence and/or concentration of various suitable molecules that react in the presence of electro-chemical/ionic binding at fluid-sensor interface 24. Example uses include small and thin form-factor biosensor applications (e.g., non-invasive/minimally invasive diagnostics procedures), such as pin-prick strips, catheters probe tips, and body patches for detection of disease markers (glucose, cardiac, cancer, neural, infection, etc.), etc.; food packaging and monitoring; etc.

Embodiments of biosensing system 10 may be included with “detect-to-warn” and “detect-to-treat” features that can perform ultra-sensitive and highly specific detection of target agents in a non-invasive manner. In some embodiments, the analysis and quantification may be performed in real-time and data transmitted using near-field communications from user to desired locations (e.g., wearable units, hand held units, personal computing devices, medical monitoring units, etc.)

Biosensor 12 may be provisioned on various types of substrate 14 (e.g., rigid and flexible such as silicon, glass, printed circuit boards, polyurethane, polycarbonate, polyamide, and polyimide, etc.) for example, to facilitate continuous and real-time detection, monitoring, and quantification of chemical and biological agents in body fluids (e.g., blood, sweat, tears, urine, saliva, etc.) Real-time detection can be performed in single-use manner or continuous-use manner using the bio sensor technology of biosensing system 10.

Note that the numerical and letter designations assigned to the elements of FIG. 1 do not connote any type of hierarchy; the designations are arbitrary and have been used for purposes of teaching only. Such designations should not be construed in any way to limit their capabilities, functionalities, or applications in the potential environments that may benefit from the features of biosensing system 10. It should be understood that biosensing system 10 shown in FIG. 1 is simplified for ease of illustration.

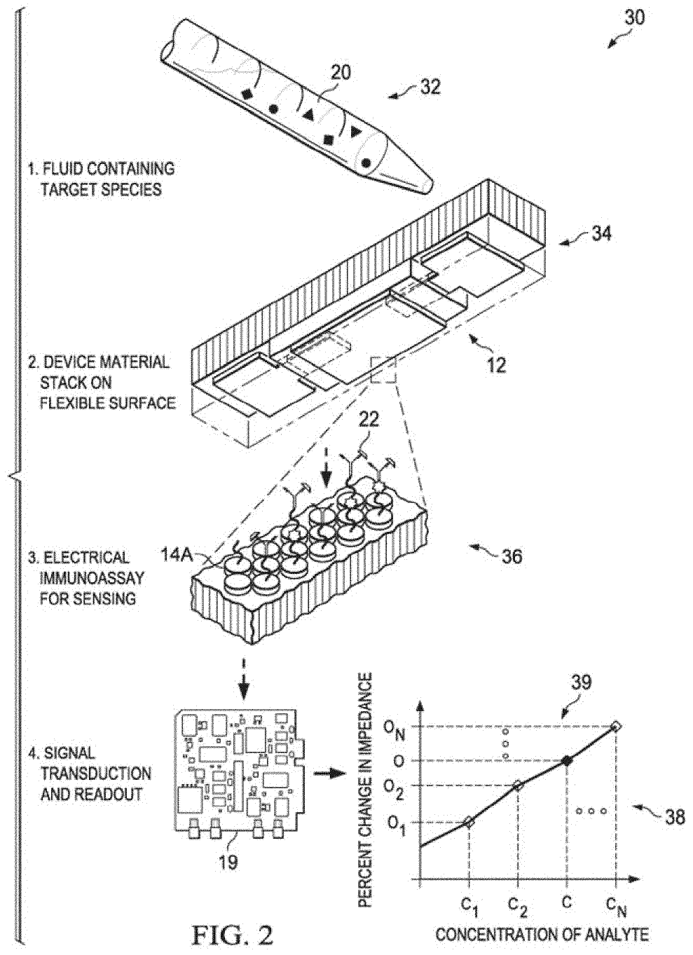

Turning to FIG. 2, FIG. 2 is a simplified block diagram illustrating example operations 30 according to an embodiment of biosensing system 10. During operation, biosensor 12 may be immersed in, or otherwise brought into contact with fluid 20 at 32. At 34, transverse voltage may be applied across transverse electrodes 18(1) and 18(2) and orthogonal voltage may be applied across orthogonal electrodes 18(1) and 18(3). The transverse voltage and orthogonal voltage generate electric fields in mutually perpendicular directions.

In a general sense, upon application of an electric field between orthogonal electrodes 18(1) and 18(3), an EDL may be generated in fluid 20. In some embodiments, the orthogonal voltage may comprise AC voltage. Such AC voltage may cause analyte 22 to additionally move normal (e.g., perpendicular, orthogonal) to the applied AC electric field direction, in a plane parallel to transverse electrodes 18(1) and 18(2), affecting the current flowing therebetween. In a general sense, the effect of AC electric fields on analyte 22 can be controlled by adjusting electric field parameters, such as amplitude, frequency, wave symmetry and phase of the AC voltage.

Further, the electric field generated by the transverse electric voltage applied across electrodes 18(1) and 18(2) may enable dielectrophoresis (DEP), in which analyte 22 is attracted to or repelled from a region of high electric field intensity in a direction perpendicular to the plane of transverse electrodes 18(1) and 18(2), thereby focusing analyte 22 at fluid-sensor interface 24. In some embodiments, an AC voltage may be initially applied across transverse electrodes 18(1) and 18(2), enabling DEP and focusing analyte 22 to fluid-sensor interface 24 at sensing element 16; subsequent switching of the voltage across transverse electrodes 18(1) and 18(2) from AC to DC, with the AC voltage across orthogonal electrodes 18(1) and 18(3) facilitates formation of the EDL in fluid 20 and modulation of current across transverse electrodes 18(1) and 18(2). Note that in some embodiments, the orthogonal voltage may also comprise DC voltage; in such embodiments, the modulation of the current between transverse electrodes 18(1) and 18(2) may not be as large as with AC voltage; nevertheless, such modulation may be sufficient to enable detection of at least a single target species of analyte 22.

At 38, a binding of analyte 22 to fluid-sensor interface 24 may be sensed (e.g., measured) through a change in impedance, capacitance, current, or voltage across sensing element 16 based on electron-ion interactions at fluid-sensor interface 24. In various embodiments, output 19 (e.g., change in impedance, current, voltage, etc.) from biosensor 12 may vary with presence, concentration and/or other characteristic of analyte 22. Output 19 may be measured using any known technique, such as potentiostat, amperometer, etc. depending on a type of output 19 (e.g., whether change in impendence, or current, etc.)

In various embodiments, biosensor 12 may be initially calibrated at 38 for a specific analyte through suitable calibration steps (e.g., fluid calibration and electronic calibration). For example, fluid 20 may comprise a liquid containing analyte 22 in a known concentration, say C₁. The transverse and orthogonal voltages may be applied across electrodes 18(1)-18(3) and output 19 measured to be, say O₁. Output 19 may comprise impedance in some embodiments, as illustrated in the figure. Output 19 may also comprise any other suitable measurement, including capacitance, current, etc. In some embodiments, O₁ may comprise response 19(2). In other embodiments, O1 may comprise a suitable combination of baseline 19(1) and response 19(2). Next, concentration of analyte 22 may be changed in fluid 20 to another known concentration, say C₂. The transverse and orthogonal voltages may be applied across electrodes 18(1)-18(3) and output 19 measured to be, say O₂. The process may be continued until a range of concentrations has been measured, from C₁ to C_{N}. A calibration chart 39 may be generated with analyte concentrations C₁, C₂, . . . C_{N} charted against corresponding outputs O₁, O₂, . . . O_{N}. Calibration chart 39 may provide an expected analyte concentration (within range C₁-C_{N}), for a known output (within range O₁-O_{N}), and vice versa. After testing with an unknown analyte concentration to obtain corresponding output 19, say O, the calibration chart may be used to obtain the corresponding analyte concentration, C, therefrom. Although one particular calibration technique has been described herein, any suitable calibration technique may be used within the broad scope of the embodiments.

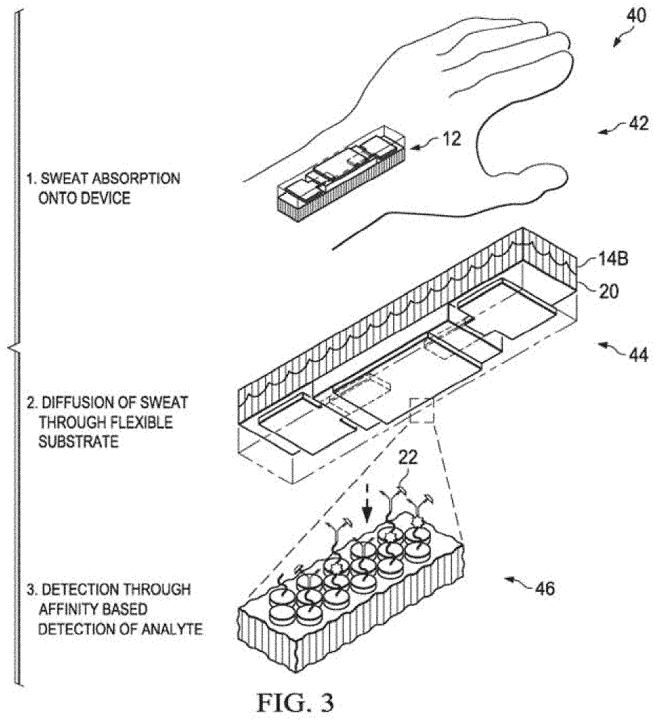

Turning to FIG. 3, FIG. 3 is a simplified block diagram illustrating example operations 40 that may be associated with an embodiment of biosensing system 10. At 42, biosensor 12 may be attached (e.g., removably, for example, using an appropriate adhesive) to skin. Sweat, comprising fluid 20, may diffuse through the porous membrane portion 14B of substrate 14 at 22. At 46, transverse and orthogonal voltages may be applied to electrodes 18(1)-18(3), resulting in electron-ion interaction between analyte 22 (e.g., salt) in fluid 20 and sensing element 16 at fluid-sensor interface 20. The interaction may be sensed through a change in output 19, which may indicate an amount of analyte 22 in the sweat.

Note that a similar procedure may be followed to measure any suitable secretion, including tears. In a general sense, sweat may be noisier than tears. Similar procedures may be followed for blood testing using a finger prick, similar to a glucose sensor; urine testing using a test strip comprising biosensor 12, similar to a typical pregnancy tester; and saliva testing with biosensor 12 inserted into a mouth guard or similar device.

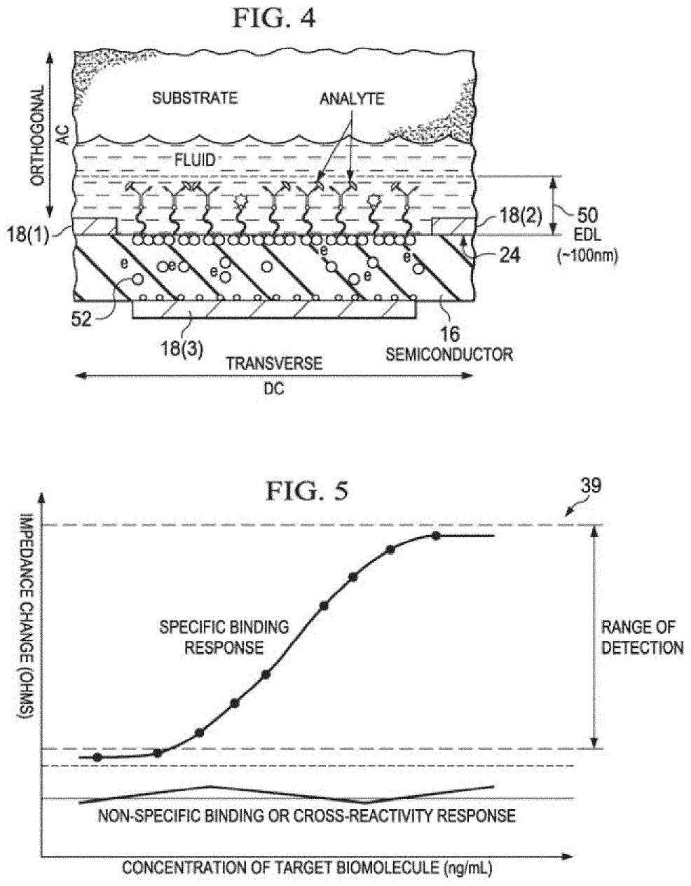

Turning to FIG. 4, FIG. 4 is a simplified block diagram illustrating example details according to an embodiment of biosensing system 10. A porous membrane of portion 14B of substrate 14 may allow fluid 20 to contact sensing element 16 along fluid-sensor interface 24 through pores 26. A transverse voltage is applied across sensing element 16 between transverse electrodes 18(1) and 18(2). An orthogonal voltage is applied across sensing element in a direction perpendicular to the transverse voltage across electrodes 18(1) and 18(3). In some embodiments, the transverse voltage is direct current (DC) voltage, whereas the orthogonal voltage is alternating current (AC) voltage.

The biological target molecule, comprised in analyte 22, reaches fluid-sensor interface 24 through portion 14B of substrate 14 and generates an electrical output based on its presence, concentration, or other characteristic. In various embodiments, the voltage across sensing element 16 modulates the electrical field at fluid-sensor interface 24, causing polarization of charges and generation of capacitance of EDL 50 in fluid 20 and capacitance of space-charge 52 (also referred to as charge depletion layer) in sensing element 16 from binding of analyte 22 to fluid-sensor interface 24. Positive or negative charges are accumulated at fluid-sensor interface 24, depending on the type of semiconductor material of sensing element 16 (e.g., n-type, p-type, steady state potential (e.g., work function differences)) and the target species (e.g., negative, positive, or neutral) comprised in analyte 22 binding to fluid-sensor interface 24. Charge modulation occurs as a result of the applied electric fields and also from modification to the bound target species; the charge modulation is measured as output 19, for example, through a change in current flow, or as impedance of the circuit.

Turning to FIG. 5, FIG. 5 is a simplified diagram illustrating an example calibration chart 39 according to an embodiment of biosensing system 10. According to calibration chart 39, the concentration of a target species (e.g., target biomolecule) may be plotted along the X-axis in ng/mL; the corresponding impedance of sensing element 16 may be plotted along the Y-axis in ohms. Note that the Y-axis can plot any suitable output, including percentage change in impedance; capacitance; etc. Detection of a specific target species of analyte 22 is achieved through an affinity based mechanism, wherein the target species binds to sensing element 16 through specific capture molecules (e.g., synthetic DNA, Peptide nucleic acid (PNA), antibodies, etc.) The affinity binding produces charge perturbation at fluid-sensor interface 24 and can be measured as output 19. Sometimes, one or more interfering species other than the target species of analyte 22 can be present in fluid 20, which can also interact with fluid-sensor interface 24. Binding of the specific target species to fluid-sensor interface 24 is referred to as specific binding response; binding of the interfering species to fluid-sensor interface 24 is referred to as cross-reactivity response (or non-specific binding response). In some embodiments, the cross-reactivity response may be subtracted out of the specific binding response through appropriate logic circuits or algorithms.

In some embodiments, the change in measured impedance may be small for small concentrations; likewise, the change in measured impedance may be small when the concentration of the target species in fluid 20 is high. In other words, biosensor 12 may be tuned to provide greater sensitivity to concentrations of target species within a specific range. Such a zone of greater sensitivity may be referred to as a range of detection. In the range of detection, small changes in concentration may correspond to relatively large changes in measured impedance. The sensitivity of biosensor 12 to the target species may be tuned using various electrode designs, material selection for sensing element 16, and other parameters based on particular needs and availability.

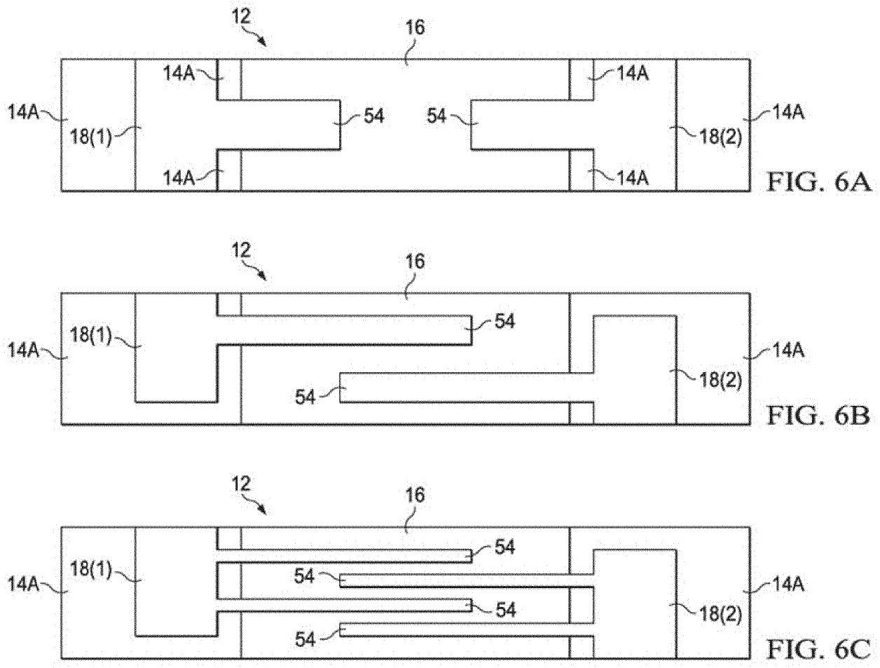

Turning to FIGS. 6A-6C, FIGS. 6A-6C are simplified block diagrams illustrating example details of biosensor 12. Electrodes 18(1)-18(3) may comprise various nanostructures in a planar dimension. Spatial patterning of electrodes 18(1)-18(3) can affect the placement and shape of the planar aggregation of analyte 22 at fluid-sensor interface 24, thereby affecting the sensitivity of biosensor 12. For example, the specific shape of electrodes 18(1) and 18(2) can affect the impedance and thereby the ionic current in fluid 20 at the vicinity of fluid-sensor interface 24.

In FIG. 6A, electrodes 18(1) and 18(2), across which the transverse voltage is applied may be situated on the same plane. Each of electrodes 18(1) and 18(2) may comprise digits 54 that may extend over sensing element 16. Digits 54 may be parallel along the length and face each other over sensing element 16. Digits 54 may be tailored for particular electrical modulation properties desired for specific target species of analyte 22. For example, in FIG. 6B, each of electrodes 18(1) and 18(2) may comprise digits 54 that are offset from each other along their widths and overlap along their lengths. In FIG. 6C, each of electrodes 18(1) and 18(2) may comprise a plurality of interleaving digits 54, overlapping along their lengths and offset along their widths. Note than any number of digits 54 (or other design features of electrodes 18(1)-18(3)) may be included within the broad scope of the embodiments.

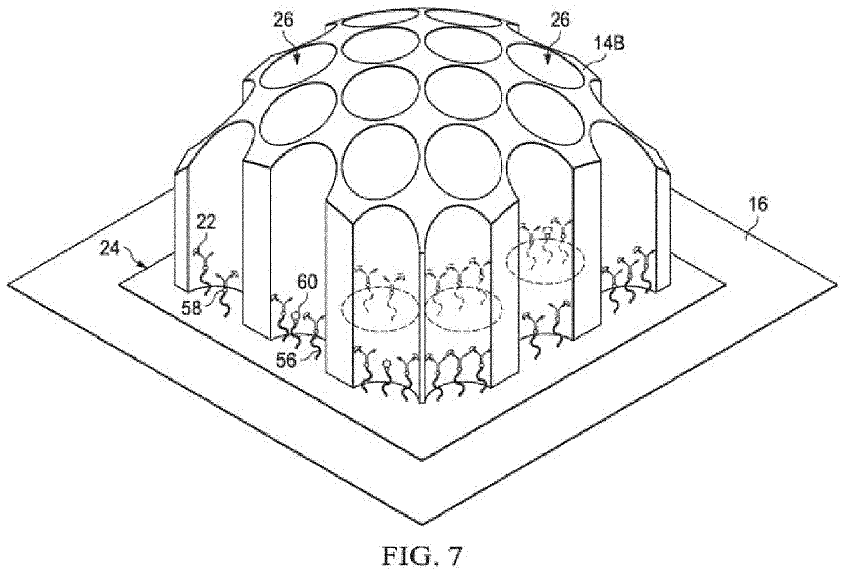

Turning to FIG. 7, FIG. 7 is a simplified block diagram illustrating example details associated with binding according to an embodiment of biosensing system 10. Based on the method and physical conditions used for deposition of sensing element 16, selective surface tuning can be achieved thereon, for example, to enhance sensitivity of biosensor 12 to specific target species of analyte 22. In an example embodiment, in which sensing element 16 comprises ZnO, enriched zinc terminated sites or enriched oxygen terminated sites may be deposited on ZnO, for example. The Zn-terminated site and the O-terminated sites can influence immobilizing of biomolecular capture probes based on preferential binding of linker molecules to either Zn-terminated sites or O-terminated sites. Such mechanisms can improve efficiency and sensitivity of biosensor 12. Note that other embodiments can include Graphene oxide or MoS₂ instead of ZnO.

In some embodiments, analyte 22 may bind directly to sensing element 16 at fluid-sensor interface 24. In other embodiments, to achieve affinity binding of target species, the surface of sensing element 16 comprising fluid-sensor interface 24 is functionalized with various linker molecules 56. Analyte 22 binds to linker molecules 56, effecting charge modulation at fluid-sensor interface 24. Dithiobis succinidyl propionate is an example of a thiol linker molecule 56. Any other suitable linker molecule, such as carboxylic molecules, hydroxyl molecules, etc. may be used within the broad scope of the embodiments.

In yet other embodiments, to achieve affinity binding of target species, the surface of sensing element 16 comprising fluid-sensor interface 24 is functionalized with various linker molecules 56, to which capture probes 58 are bound. Although additional molecules may be bound to capture probes 58, and so on, beyond three levels of binding, the charge modulation and characterization signal may become weak and difficult to isolate from noise, affecting sensitivity of biosensor 12. Capture probes 58 may connect linker molecules 56, which are attached to the surface of sensing element 16, to analyte 22. Examples of capture probes 58 includes aptamers, antibodies, enzymes, peptides, and amino acid and nucleic acid sequences.

In some embodiments, blocking molecules 60 may neutralize linker molecules 56 without capture probes 58 and minimize binding of interfering species that can cause signal attenuation and cross-reactivity responses. Examples of blocking molecules 60 include SuperBlock™, albumin based solutions that can block unbound organic—NH groups on linker molecules 56.

Note that for ease of illustration, pore 26 is illustrated as a through-hole pore. Various other pore configurations may be included within the broad scope of the embodiments. For example, pore 26 may comprise intercalated pores in some embodiments. In other embodiments, pore 26 may comprise hierarchical pores (e.g., pore in pore). Various commercially available materials may be used to fabricate substrate 14 to include suitable pore configurations. For example, Merocel™ is one of a commercially available porous material of the intercalated “sponge” like material. Other commercially available materials include those sold by Whatman Membranes™ from GE Life Sciences™, Advantec™, etc. Hierarchical pores may be seen in diatoms, and similar pore configuration used in synthesizing appropriate substrate materials.

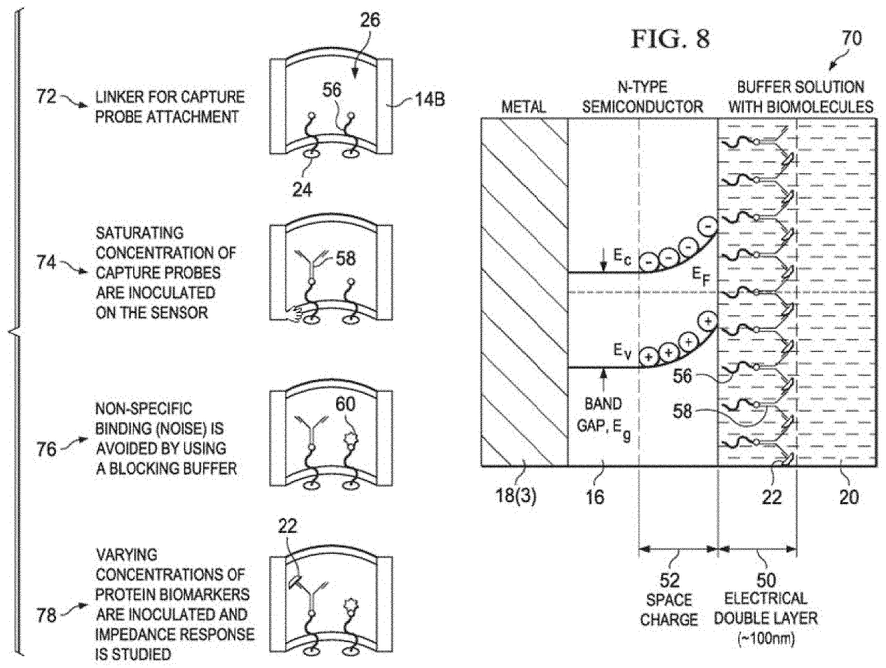

Turning to FIG. 8, FIG. 8 is a simplified diagram illustrating example operations 70 and details according to an embodiment of biosensing system 10. At 72, linker molecule 56 (e.g., dithiobis succinimidyl propionate) may be bound to the surface of sensing element 16 at fluid-sensor interface 24, comprising the surface of sensing element 16 that can be exposed to fluid 20. At 74, a saturating concentration (e.g., maximum concentration loadable onto sensing element 16) of capture probes 58 may be inoculated (e.g., introduced, such as with a fixed volume of solution, in a metered manner) on biosensor 12. Capture probes 58 bind to at least some linker molecules 56. At 76, non-specific binding noise may be avoided by adding a blocking buffer containing blocking molecules 60. At 78, varying concentrations of analyte 22 (e.g., protein biomarkers) are inoculated and impedance response is studied to generate calibration chart 39.

An example cross-section is also shown, comprising gold electrode 18(3) in Ohmic contact with n-type metal oxide semiconductor sensing element 16, which is in contact with fluid 20. The electrical voltage supplied to sensing element 16 may provide sufficient energy to polarize fluid 20 and activate sensing element 16. Charge depletion layer 52 and electrical double layer 50 may vary in size and electrical properties based on the binding interaction between analyte 22 and sensing element 16 at fluid-sensor interface 24. Note that charge depletion layer 52 indicates modulation of electro-ionic charge distribution at fluid-sensor interface 24. In the example embodiment shown, sensing element 16 is a n-type semiconductor and at steady state, the energy bands of the semiconductor material of sensing element 16 are bent to align Fermi levels, resulting in charge build-up on the semiconductor side of fluid-sensor interface 24. Charge depletion layer 52 may also be referred to in this Specification as the “space charge” region. Conversely, for a p-type semiconductor material of sensing element 16, the space charge region is formed by energy band bending in the opposite direction. In a general sense, the space charge region forms in response to distortion of the semiconductor material's valence and conduction bands (“band bending”) in the vicinity of fluid-sensor interface 24.

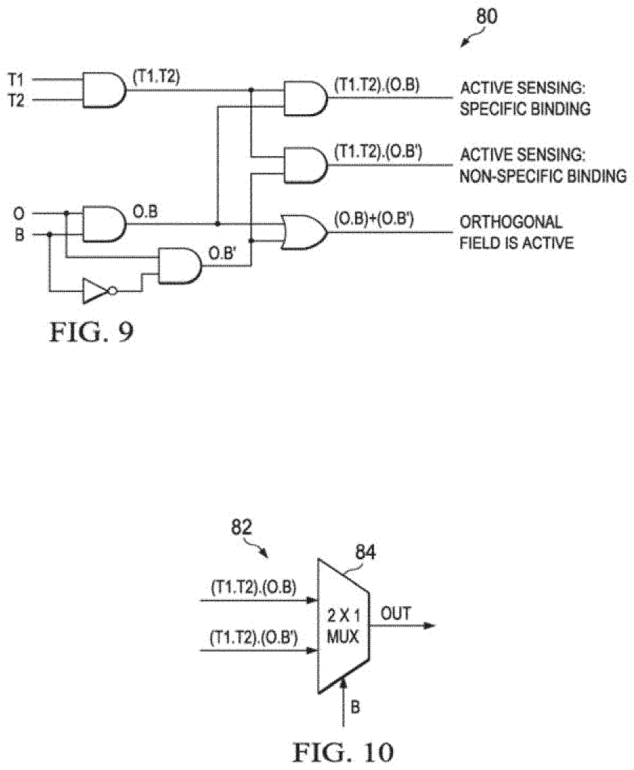

Turning to FIG. 9, FIG. 9 is a simplified circuit diagram illustrating example details of a digital logic circuit 80 representing an embodiment of biosensing system 10. Digital logic circuit 80 facilitates communicating output 19 in a user readable format. In some embodiments, digital logic circuit uses digitized analog signal as inputs. T1 and T2 represent an electrode pair (e.g., electrodes 18(1) and 18(2)) applying an analog voltage signal to biosensor 12. O represents the orthogonal voltage input by electrode 18(3), and corresponds to a clock/power signal indicating the operational state of biosensor 12. B represents the signal corresponding to modulation to the electric field at fluid-sensor interface 24 from a specific target species; the B signal may be digitized to 0 to indicate absence of the target species and 1 to indicate presence of the target species. B′ represents the signal due to any non-specific molecule or binding in the absence of the target species. The output from digital logic circuit 80 may be input to a microcontroller (not shown).

Digital logic circuit 80 makes a decision regarding the presence or absence of the target species based on user-defined (e.g., predetermined) inputs T1,T2 and O and measured signal B. Output (T1.T2).(O.B) represents active sensing of binding of target species with a 0 signal indicating absence of the target species, and a 1 signal indicating presence of the target species; output (T1.T2).(O.B′) represents active sensing of binding of any non-target species with a 0 signal indicating absence of the non-target species, and a 1 signal indicating presence of the non-target species; and output (O.B).(O.B′) represents whether the orthogonal voltage field is active, with a 0 signal indicating that the field is not active, and a 1 signal indicating that the field is active.

Turning to FIG. 10, FIG. 10 is a simplified circuit diagram illustrating example details of a digital logic circuit 82 representing an embodiment of biosensing system 10. In some embodiments, a multiplexer 84 (e.g., an integrated circuit) may be used for decision making based on outputs of digital logic circuit 80. Output (T1.T2).(O.B) of digital logic circuit 80 may be input as 1 signal to multiplexer 84 when specific binding from target species occurs is detected and as 0 signal when non-specific binding occurs. Likewise, output (T1.T2).(O.B′) of digital logic circuit 80 may be input as 1 when target species is not present and as 0 when target species is detected. An OUT signal is output from multiplexer; the OUT signal can digital 0 or 1 based on the presence or absence of the target species.

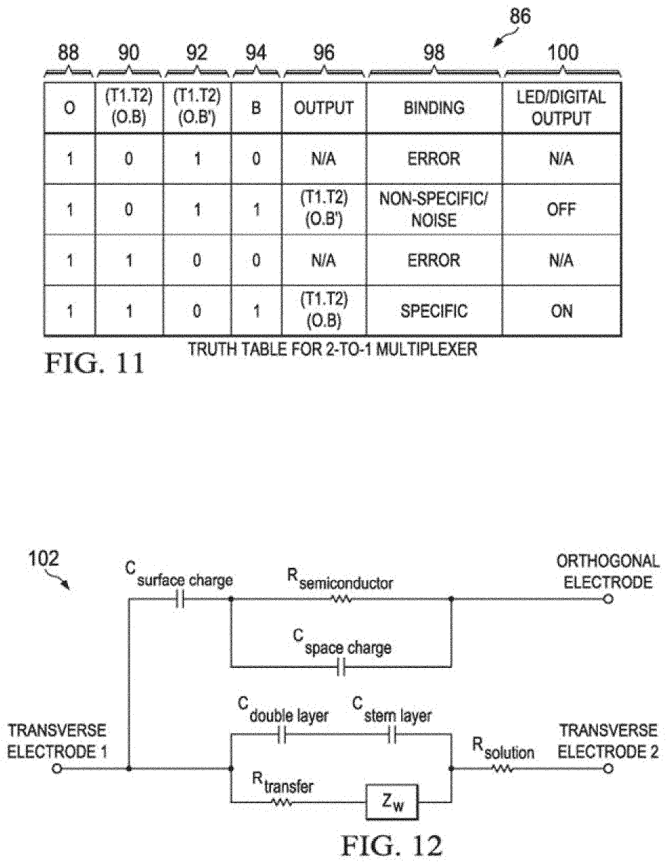

Turning to FIG. 11, FIG. 11 is a simplified diagram illustrating an example truth table 86 for multiplexer 84 of digital logic circuit 82 according to an embodiment of biosensing system 10. Column 86 represents a clock/power signal indicating the operational state of biosensor 12. Column 90 represents active sensing of binding of target species with a 0 indicating absence of the target species, and a 1 indicating presence of the target species. Column 92 represents active sensing of binding of any non-target species with a 0 indicating absence of the non-target species, and a 1 indicating presence of the non-target species. Column 94 represents the signal corresponding to modulation to the electric field at fluid-sensor interface 24 from a specific target species; the B signal may be digitized to 0 to indicate absence of the target species and 1 to indicate presence of the target species.

Column 96 represents the OUT signal of multiplexer 84 to the corresponding inputs as indicated in columns 88-94. Column 98 represents an interpretation by a microprocessor based on inputs and processed signal out from biosensor 12. The OUT signal from multiplexer 84 can be used to turn on a light emitting diode (LED) or other suitable display or indicator. The status of the indicator corresponding to the various inputs and outputs as indicated in respective rows is represented in column 100. Thus, according to truth table 86, if the orthogonal voltage has been turned on (O=1), and the target species is detected (B=1; (T1.T2)(O.B)=1), along with absence of non-target species ((T1.T2)(O.B′)=0), the LED light turns on (LED output=ON).

Turning to FIG. 12, FIG. 12 is a simplified diagram illustrating an example circuit model 102 according to an embodiment of biosensing system 10. Equivalent circuit model 102 can be used to represent the three electrode configuration of biosensor 12 and comprehend output 19 comprising an analog signal measured at electrodes 18(1)-18(3). C_{surface} charge represents capacitance of the surface charges at fluid-sensor interface 24; R_{semiconductor} represents resistance of sensing element 16; and C_{space charge} represents capacitance of charge depletion layer 52. C_{surface charge}, R_{semiconductor} and C_{space charge} influence signal output from biosensor 12 at orthogonal electrode 18(3). C_{double layer}, represents the capacitance of EDL 50; C_{stern layer} represents capacitance of the Stern layer in fluid 20; R_{transfer} and Z_{w} represent resistive parameters of fluid 20 at fluid-sensor interface 24 that influence signal output from biosensor 12 as measured at transverse electrodes 18(1) and 18(2).

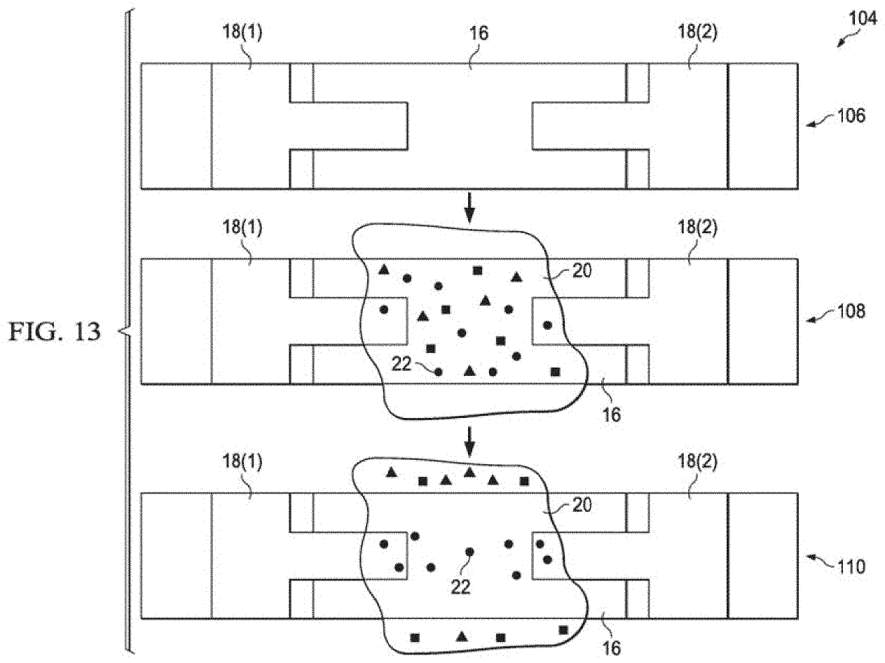

Turning to FIG. 13, FIG. 13 is a simplified diagram illustrating example operations 104 associated with an embodiment of biosensing system 10. Use of ultra-low sample volumes (e.g., less than 100 microliters) can cause non-uniform distribution of analyte 22 at fluid-sensor interface 24, resulting in high noise and spurious artifacts in the measured output signal. In various embodiments, electrokinetic focusing may be used to direct charged or uncharged species of analyte 22 to a specific region of fluid-sensor interface 24 at sensing element 16, thereby reducing the non-uniform distribution of analyte 22. ‘Electrokinetic focusing’ as used in this Specification refers to using electrokinetic transport (e.g., electrophoretic migration of ions) to enable spatial confinement of fluid 20 and analyte 22 to fluid-sensor interface 24 at sensing element 16. Electrokinetic focusing can reduce detection time and enable the detection of charged and uncharged target species of analyte 22. In various embodiments, particle oscillation from gradient electric fields and dielectrophoresis (DEP) are used to effect electrokinetic focusing.

In some embodiments, a gradient electric field is applied transversely across electrodes 18(1) and 18(2) on the same (e.g., X-Y) plane. The gradient electric field causes local polarization of fluid 20 and target species of analyte 22, driving analyte 22 towards fluid-sensor interface 24 at sensing element 16. Under the influence of the gradient electric field, analyte 22 undergoes polarization (note that analyte 22 can be charged or uncharged prior to the application of the gradient electric field). The gradient electric field orthogonal to electrodes 18(1) and 18(2) within a microenvironment (e.g., a few 100 nm around each molecule of analyte 22) of analyte 22 causes polarization of analyte 22. The polarization and particle oscillation can be affected by the harmonic or resonance frequencies associated with the fluctuating gradient electric field. Such particle oscillation effects are compatible with single- and multi-phase sinusoidal voltage.

According to DEP, a force is exerted on any dielectric particle when it is subjected to a non-uniform electric field. In a general sense, all dielectric particles exhibit dielectrophoretic activity in the presence of electric fields; however, the strength of the force depends strongly on the medium and particles' electrical properties, on the particles' shape and size, and on the frequency of the electric field. Consequently, fields of a particular frequency can manipulate specific particles with relatively greater selectivity. DEP and particle oscillation are used to achieve targeted spreading of analyte 22 uniformly on sensing element 16.

At 106, a transverse voltage is applied across electrodes 18(1) and 18(2), causing a gradient electric field around them. At 108, a small volume (e.g., less than 1-10 microliters) of fluid 20 including analyte 22 is introduced on sensing element 16. In the absence of electrokinetic focusing, the movement of analyte 22 to fluid-sensor interface 24 at sensing element 16 is diffusion driven, which can be slow and non-uniform. At 110, under electrokinetic focusing based on particle oscillation from gradient electric fields and DEP, analyte 22 is spatially contained to a small, uniform region on sensing element 16, facilitating low noise measurements from biosensor 12. In some embodiments, electrokinetic focusing may be used together with impedance spectroscopy (e.g., measurement of dielectric properties of fluid 20 as a function of voltage frequency) to detect and measure analyte 22 in fluid 20 with biosensor 12.

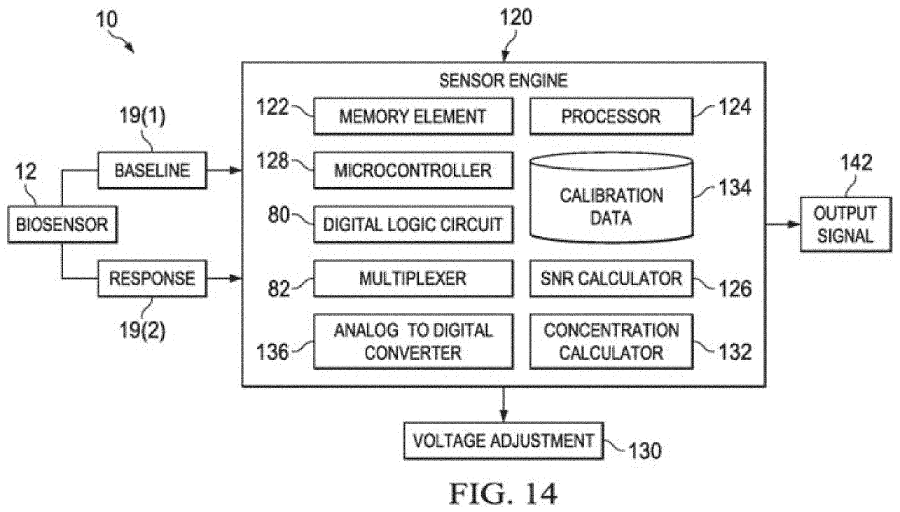

Turning to FIG. 14, FIG. 14 is a simplified block diagram illustrating example details of biosensing system 10. Baseline 19(1) and response 19(2) from biosensor 12 may be fed to a sensor engine 120. Sensor engine 120 comprises a memory element 122 and a processor 124. A SNR calculator 126 in sensor engine 120 compares baseline 19(1) and response 19(2) and determines the SNR of the measurements. A microcontroller 128 may generate a voltage adjustment 130 to orthogonal voltage across orthogonal electrodes 18(1) and 18(3) to vary the SNR. Voltage adjustment is continued until a maximum SNR is achieved. A concentration calculator 132 may compare response 19(2) with stored calibration data 134 to estimate analyte concentration corresponding to measured response 19(2). In some implementations, concentration calculator may utilize machine learning models and generate calculations from an output of the machine learning model, such as discussed herein. In various embodiments, stored calibration data 134 can comprise calibration chart 39 of the foregoing figures. In some embodiments, the calculated analyte concentration may be verified and transmitted to an external device, such as a tethered wireless display.

An analog-to-digital converter (ADC) 136 in sensor engine 120 may digitize baseline 19(1) and response 19(2) and feed the digital signals to digital circuit logic 80. In some embodiments, digitized response 19(2) may correspond to {T1, T2} of the foregoing figures and digitized baseline 19(1) may correspond to {O, B} of the foregoing figures. Digital circuit logic 80 may transform the digital signals to an output that is fed to multiplexer 82 in sensor engine 120. Multiplexer 82 may generate an output signal 142 depending on the values from digital circuit logic 80. Output signal 142 may light up an LED, or generate other suitable displays accordingly.

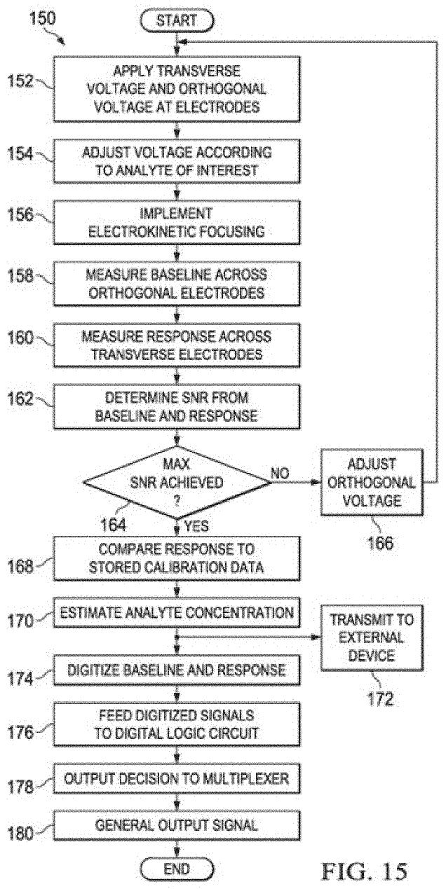

Turning to FIG. 15, FIG. 15 is a simplified flow diagram illustrating example operations 150 that may be associated with an embodiment of biosensing system 10. At 152, an input transverse voltage is applied to transverse electrodes 18(1) and 18(2) and an input orthogonal voltage is applied to orthogonal electrodes 18(1) and 18(3), the orthogonal voltage creating an electric field that is orthogonal to the electric field created by the transverse voltage. In some embodiments, the transverse voltage may comprise DC voltage, and the orthogonal voltage may comprise AC voltage. In some embodiments, instead of voltage, current may be applied across electrodes 18(1) and 18(3) to generate the transverse and orthogonal electric fields. In yet other embodiments, a steady state potential may be applied across electrodes 18(1) and 18(3) to general the transverse and orthogonal electric fields. In various embodiments, a microcontroller or microprocessor may be used to adjust a gain of biosensor 12. For example, a ratio of output signals to input voltage may be calculated to determine the gain of biosensor 12, with higher gain indicating higher sensitivity in some embodiments. The input voltages (or current, or steady state potential) may be adjusted accordingly to obtain better gain of biosensor 12.

At 154, voltage and frequency range of the AC voltage may be adjusted according to analyte 22 of interest in fluid 20. For example, sensitivity of biosensor 12 may be large for a particular target species at a specific combination of voltage amplitude and frequency of the AC voltage—in other words, biosensor 12 can detect small variations in concentrations of the particular target species at the specific combination of voltage amplitude and frequency of the AC voltage. The sensitivity may change if the target species changes, or the combination of voltage amplitude and frequency of the AC voltage changes. Conversely, biosensor 12 may detect a different target species with a different combination of voltage amplitude and frequency of the AC voltage. The sensitivity variation with voltage amplitude and frequency may be determined apriori; in some embodiments, biosensor 12 may be preconfigured to operate at a specific combination of voltage amplitude and frequency to detect a particular target species.

At 156, electrokinetic focusing may be optionally implemented through transverse electrodes 18(1) and 18(2), for example, adjusting the electric field generated by the transverse voltage to cause particle oscillation and dielectrophoretic effects on analyte 22 in fluid 20. At 158, baseline 19(1), for example, impedance, or capacitance, or current may be measured across orthogonal electrodes 18(1) and 18(3). At 160, response 19(2), for example, impedance, or capacitance, or current may be measured across transverse electrodes 18(1) and 18(2). At 162, response 19(2) may be compared to baseline 19(1) to determine the SNR of the measurements. In some embodiments, electrokinetic focusing may be performed after determining SNR; if the SNR is lower than a predetermined threshold, electrokinetic focusing may be performed, and otherwise, it may be neglected.

In some embodiments, tuning (e.g., adjusting) the height (e.g., thickness) of fluid-sensor interface 24 (e.g., Debye length tuning, EDL tuning) during electrokinetic focusing is achieved with orthogonal electrodes 18(1) and 18(3) (e.g., by varying a voltage, current, or steady state potential across electrodes 18(1) and 18(3) until a desired Debye length measurement is achieved). The height tuning enhances the target species attraction to fluid-sensor interface 24 and adds to the gradient electric field effect from transverse electrodes 18(1) and 18(2). At 164, a determination may be made whether maximum SNR is achieved. If not, the operations step to 166, at which the orthogonal voltage is adjusted and the operations repeated until maximum SNR is achieved.

At 168, response 19(2) is compared to stored calibration data 134. At 170, the analyte concentration is estimated based on calibration data 134. For example, calibration data 134 may comprise calibration chart 39. Response 19(2) may be plotted against various known analyte concentrations in calibration chart 39. A specific value of response 19(2) obtained at operation 160 may be plotted on calibration chart 39, and the corresponding analyte concentration estimated therefrom. At 172, the estimated analyte concentration may be verified and transmitted to an external end-user device, such as a computer, server, smartphone, display, etc.

Alternatively, or additionally, at 174, baseline 19(1) and response 19(2) may be digitized by ADC 136. At 176, the digitized signals may be fed to digital logic circuit 80. At 178, decisions output by digital logic circuit 80 may be fed to multiplexer 82. At 180, multiplexer 82 may generate output signal 142 depending on the input from digital circuit logic 80. Output signal 142 may light up an LED, or generate other suitable displays accordingly.

Embodiments of biosensing system 10 described herein may be used in myriad applications. Note that the electron-ion mechanism of sensing may remain constant across the different applications, whereas linker molecules 56 and capture probes 58 for binding with analyte 22 may vary across the different applications. For example, some embodiments of biosensing system 10 may be used in skin-graft sensors. Portion 14B of substrate 14 may comprise a flexible, nanoporous membrane, which may be placed in direct contact with the skin and used for continuous, periodic monitoring of various molecules present in perspired sweat by the wearer. The information collected can be used to understand body response and behaviors, for example, to aid in disease diagnosis under various situations such as outpatient, inpatient, post-surgical etc.

In another example, some embodiments of biosensing system 10 may be used in smart catheters. Miniaturized catheters in microscale may be used for continuous drug delivery and in-vivo monitoring of injuries to blood vessels, tissues etc. Flexible, nanoporous biosensors as described herein can be integrated inside the catheters to perform biochemical detection, for example, to quantitatively study the molecular environment surrounding damaged, under-treatment tissue or blood vessel of interest. In addition, the microscale nature of biosensor 12 can enable analysis of inflation pressure, upstream blood pressure and downstream blood pressure.

In yet another example, some embodiments of biosensing system 10 may be used in smart tissue sensors. The sensor platform as described herein can be used for continuously monitoring tissue development and growth without interfering with the tissue itself. Patterned and controlled growth of semiconductor nanostructures arrays (such as ZnO) can be used to create conformal and biomimetic architectures that favor growth of tissue and other structural biological elements. Biosensor 12 can be integrated with semiconductor nanostructure arrays to continuously monitor the rate of growth, biochemical environment and the influence of catalysts on tissue development.

In yet another example, some embodiments of biosensing system 10 may be used in smart food sensors. Biosensor 12 as described herein can be used for real-time monitoring of packaged food quality. Various appropriate linker molecules 56 and capture probes 58 that bind with specific food breakdown byproducts released at very low concentrations may be used to estimate the quality of the packaged food. Some embodiments of biosensor 12 may be implemented in simple household food packages, which can include plastic covers and other sealable materials, as well as industrial grade food packaging processes.

In yet another example, some embodiments of biosensing system 10 may be used in bacterial sensors and/or smart bottles. The detection of bacterial quantity and type in water, milk, etc. can establish its safety and usability levels for consumption. Biosensor 12 described herein may be conjugated with nucleic acid vectors or capture probes 58 that can detect cyanobacteria, algae and other classes of bacteria that make the fluids unsafe for consumption. Biosensor 12 can be integrated onto a bottle used for collecting/storing the fluid (examples: water, milk, baby products, etc.) In yet another example, some embodiments of biosensing system 10 may be built on contact lens polymeric materials to detect biochemical markers in tears to quantitatively evaluating glaucoma and diabetes. In yet another example, some embodiments of biosensing system 10 may be used in a blood prick sensor for cancer detection, vascular disease detection, etc. In yet another example, some embodiments of biosensing system 10 may be used in urine testing strips for cancer detection. In yet another example, some embodiments of biosensing system 10 may be integrated into a mouth guard to test saliva for disease detection.

Note that only a few example applications are described herein; various other applications using integrated sensors within wearable or flexible fabric materials and other substrates may be included within the broad scope of the embodiments. Integrated sensors may also be envisioned within medical instruments such as catheters, probes, patches for non-communicable disease diagnosis such as cardiac, cancer, Alzheimer's, etc.

Some embodiments of biosensing system 10 as described herein provides rapid analyte detection and/or sensor devices and methods of use thereof in the identification of a binding event. Such methods find application in inter alia, immunoassays, screening assays, enzymatic assays, diagnostic assays, screening assays, assays for the identification of biological and/or environmental toxins, and others, as will be appreciated by one skilled in the art.

In various embodiments, nanostructures on the biosensor surface (e.g., surface of sensing element 16 proximate fluid-sensor interface 24) can be formed under controlled manufacturing conditions consistent with microchip scale and photomask processes, for example, to produce highly uniform and/or miniaturized and/or high-density array sensor devices. Biosensor 12 described herein may also be fabricated via microfabrication technology, or microtechnology, in one embodiment, applying the tools and processes of semiconductor fabrication to the formation of, for example, physical structures, such as electrodes 18(1)-18(3) and sensing element 16. Microfabrication technology allows for example, to precisely design features (e.g., wells, channels) with dimensions in the range of <1 mm to several centimeters on chips made, for example, of silicon, glass, or plastics. In some embodiments, NEMS or nanotechnology, for example, using nanoimprint lithography (NIL), may be used to construct the devices described herein.

According to various embodiments, biosensor 12 described herein may be adapted such that analysis of a species of interest may be conducted, in one embodiment, in biosensor 12 described herein, or in another embodiment, downstream of biosensor 12 described herein, for example, in a separate server coupled to the device. It is to be understood that the devices described herein may be useful in various analytical systems, including bioanalysis microsystems. Although the biosensor system has been described with respect to particular devices and methods, it will be understood that various changes and modifications can be made without departing from the scope of the embodiments.

In another example, a sensing device is provided for detecting one or more target analytes in a fluid sample. The sensing device may include a substrate comprising two or more electrodes. A plurality of semiconducting nanostructures may be disposed on at least one of the electrodes. A plurality of capture reagents may be attached to the plurality of semiconducting nanostructures. The plurality of capture reagents are configured to selectively bind to the one or more target analytes in the fluid sample, thereby effecting changes to electron and ion mobility and charge accumulation in different regions of the semiconducting nanostructures and the fluid sample. The changes to the electron and ion mobility and charge accumulation are detectable with aid of sensing circuitry, and can be used to determine a presence and concentration of the one or more target analytes in the fluid sample.

Embodiments of the present disclosure are also directed to a method of detecting one or more target analytes in a fluid sample. The method may include providing a sensing device comprising (1) a substrate comprising two or more electrodes, (2) a plurality of semiconducting nanostructures disposed on at least one of said electrodes, and (3) a plurality of capture reagents attached to the plurality of semiconducting nanostructures. The method may include applying the fluid sample containing the one or more target samples to the sensing device. Additionally, the method may include detecting, with aid of sensing circuitry, changes to electron and ion mobility and charge accumulation in different regions of the semiconducting nanostructures and the fluid sample when the plurality of capture reagents selectively bind to the one or more target analytes in the fluid sample. The method may further include determining a presence and concentration of the one or more target analytes based on the detected changes to the electron and ion mobility and charge accumulation.

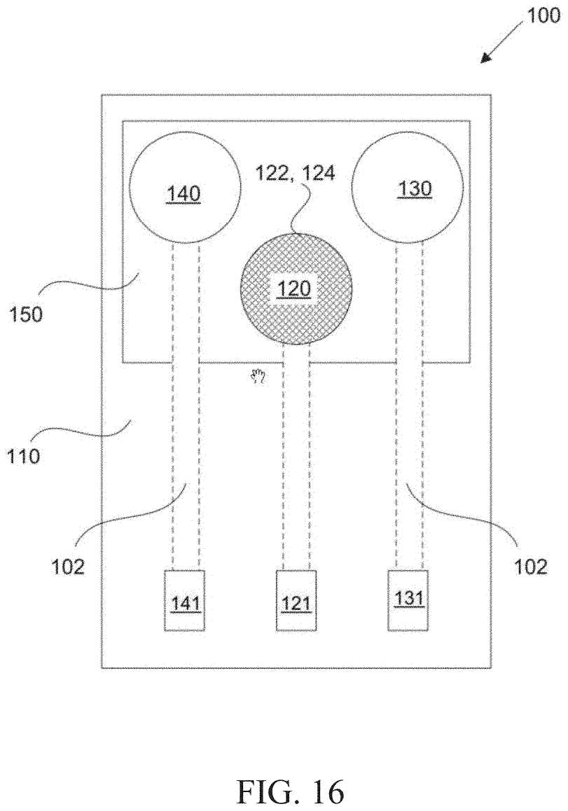

FIG. 16 shows a schematic of a sensing device 100 in accordance with some embodiments. The sensing device 100 may be used to conduct one or more immunoassays for detecting one or more target analytes in a sample. The sensing device may contain a plurality of capture reagents for conducting the one or more immunoassays. The capture reagents may be disposed or immobilized on a surface of at least one electrode of the sensing device. Generally, the sensing device comprises materials suitable for performing biosensing, by providing appropriate materials for immobilizing or otherwise providing various capture reagents to perform the immunoassay.

Referring to FIG. 16, the sensing device 100 may comprise a substrate 110. The substrate may be flexible or rigid. The substrate may include materials such as polyimide, silicon, glass, printed circuit boards (PCB), polyurethane, polycarbonate, polyamide, or the like. In some embodiments, the substrate may be an organic substrate comprising flexible PCB materials. In some embodiments, the substrate may be a flexible and porous polyimide substrate that allows very low volumes of fluid adsorption within its pores, which in turn facilitates more effective conjugation and thus improved sensitivity in the detection of one or more target analytes present in the fluid sample. In some embodiments, the substrate may be capable of flexing or bending a large number of cycles without substantially impacting the accuracy and sensitivity of the sensing device.

In some embodiments, the substrate may comprise test strips for aiding lateral transport of a sample fluid to electrodes on the sensing device. Non-limiting examples of test strips may include porous paper, or a membrane polymer such as nitrocellulose, polyvinylidene fluoride, nylon, Fusion 5™, or polyethersulfone.

In some embodiments, the sensing device may be provided on a single electrochemical test strip. For example, the sensing device need not include multiple electrochemical test strips for performing the simultaneous and multiplexed detection of a plurality of target analytes.

The sensing device 100 may comprise two or more electrodes disposed on the substrate. For example, in the embodiment shown in FIG. 16, a working electrode (WE) 120, a reference electrode (RE) 130, and a counter electrode (CE) 140 may be disposed on the substrate 110. Any number or type of electrodes may be contemplated. The electrodes may be exposed to a sample suspected to contain one or more target analytes. A working electrode (WE) as described anywhere herein may be referred to interchangeably as a sensing electrode, a sensing working electrode, detection electrode, or the like. The WE 120 may comprise a conducting electrode stack. The WE 120 may further comprise a semiconducting sensing element (e.g., a plurality of semiconducting nanostructures 122) formed on its surface, as described in detail elsewhere herein. The RE 130 and CE 140 may each comprise a conducting electrode stack, and need not comprise sensing elements on their surfaces. For example, the RE 130 and CE 140 need not include molecules that are used for functionalizing the sensing element on the WE 120. The CE 140 and RE 130 may be electrochemically inert/stable, and may collectively form an electrochemical cell with the WE 120 when the electrodes come into contact with the fluid sample (electrolyte or ionic liquid).

The electrodes may be formed of various shapes and/or sizes. The electrodes may have a substantially circular or oval shape, for example as shown in FIG. 16. In some embodiments, the electrodes may have a regular shape (e.g. polygonal shapes such as triangular, pentagonal, hexagonal, etc.) or an irregular shape. The electrodes may be of the same size or different sizes. The electrodes may have the surface areas or different surface areas. The ratio of the surface areas of WE:CE:RE may be given by x:y:z, where x, y and z may be any integer. In some instances, z may be larger than x and y, such that the RE 130 has a larger surface area than each of WE 120 and CE 140. For example, the ratio of the surface areas of WE:CE:RE may be 1:1:2, 1:1:3, 1:1:4, 1:1:5, 1:1:6, or any other ratio. In some preferred embodiments, the ratio of the surface areas of WE:CE:RE may be 1:1:4, but is not limited thereto.

The electrodes on the sensing device 100 may be electrically connected to a plurality of contact pads via conducting layer traces 102 embedded or formed on the substrate. Each electrode may be connected to a contact pad. For example, the working electrode 120 may be connected to a first contact pad 121, the reference electrode 130 may be connected to a second contact pad 131, and the counter electrode 140 may be connected to a third contact pad 141. In some alternative embodiments, two or more electrodes may be connected to a contact pad. Optionally, an electrode may be connected to two or more contact pads. The contact pads may be located at a distance from the electrodes. In some embodiments, the contact pads and electrodes may be located at opposite ends of the substrate. The contact pads may be provided on a same surface of the substrate 110 as the electrodes. Alternatively, the contact pads may be provided on a different surface of the substrate 110 as the electrodes. For example, the contact pads and the electrodes may be provided on opposite surfaces of the substrate.

The conducting layer traces 102 may be formed of a metal, e.g. Cu. The electrodes 120, 130, and 140 may include a surface finish formed on the conducting layer traces. Non-limiting examples of surface finishes may include electroless nickel deposited on a copper trace, or an immersion gold/immersion silver/electrolytic gold deposited on an electroless nickel surface.

In some embodiments, different surface finishes on a flexible printed circuit board substrate may comprise the following exemplary thickness ranges: (1) For Immersion Silver, 8-15 micro-inches of 99% pure silver over Cu trace layer with good surface planarity, which may be a preferred surface finish for RE 130. In some cases, the post immersion silver surface finish may be chemically modified to form an Ag/AgCl surface that offers excellent electrochemical stability. (2) For Electroless Nickel Immersion Gold (ENIG), 2-8 micro-inches Au layer over 120-240 micro-inches electroless Ni layer over Cu trace layer. (3) For Electroless Nickel Electroless Palladium Immersion Gold (ENEPIG), 2-8 micro-inches Au layer over 4-20 micro-inches electroless Pd layer over 120-240 micro-inches electroless Ni layer. The Pd layer can eliminate corrosion potential from immersion reaction. Au surfaces are relatively stable/inert, offer wide electrochemical window and can be used for the WE 120 and CE 140. It should be appreciated that the above thickness values are merely exemplary, and that different thickness values may be contemplated for different surface finishes depending on the desired electrical and sensing properties.

Semiconducting nanostructures may be disposed on at least one of the electrodes to aid in sensing of one or more target analytes. For example, a sensing element comprising a layer of semiconducting nanostructures 122 may be deposited over the surface of the WE 120. The WE 120 may include one or more of the surface finishes described herein. The choice of semiconducting nanostructures 122 may be determined based on the catalytic properties of the semiconducting material. In some embodiments, metal oxide nanostructured surfaces can offer immobilization when selectively functionalized with thiol and phosphonic acid linker chemistries to form specific interactions with the protein biomolecules, that can lead to enhancements in specific output signal response and enhanced specificity in biomarker detection.

Non-limiting examples of semiconducting materials that can be used on a working electrode may include the following: Diamond, Silicon, Germanium, Gray tin (αSn), Sulfur (αS), Gray selenium, Tellurium, Silicon carbide (3CSiC), Silicon carbide (4HSiC), Silicon carbide (6HSiC), Boron nitride (cubic), Boron nitride (hexagonal), Boron nitride (nanotube), Boron phosphide, Boron arsenide, Aluminium nitride, Aluminium phosphide, Aluminium arsenide, Aluminium antimonide, Gallium nitride, Gallium phosphide, Gallium, arsenide, Gallium antimonide, Indium nitride, Indium, phosphide, Indium arsenide, Indium antimonide, Cadmium selenide, Cadmium, sulfide, Cadmium telluride, Zinc oxide, Zinc selenide, Zinc sulfide, Zinc telluride, Cuprous, chloride, Copper sulfide, Lead selenide, Lead(II) sulfide, Lead telluride, Tin sulfide, Tin sulfide, Tin telluride, Bismuth, telluride, Cadmium phosphide, Cadmium arsenide, Cadmium antimonide, Zinc phosphide, Zinc arsenide, Zinc antimonide, Titanium dioxide (anatase), Titanium dioxide (rutile), Titanium dioxide (brookite), Copper(I) oxide, Copper(II) oxide, Uranium, dioxide, Uranium, trioxide, Bismuth, trioxide, Tin dioxide, Lead(II) iodide, Molybdenum disulfide, Gallium, selenide, Tin sulfide, Bismuth sulfide, Iron(II) oxide, Nickel(II) oxide, Europium(II) oxide, Europium(II) sulfide, Chromium(III) bromide, Arsenic sulfideOrpiment, Arsenic sulfideRealgar, Platinum, silicide, Bismuth(III) iodide, Mercury(II) iodide, Thallium(I) bromide, Silver sulfide, Iron disulfide, Lead tin, telluride, Thallium tin telluride, Thallium germanium telluride, Barium titanate, Strontium, titanate, Lithium niobate, Lanthanum copper oxide, Gallium manganese arsenide, Indium manganese arsenide, Cadmium manganese telluride, Lead manganese telluride, Copper indium selenide (CIS), Silver gallium sulfide, Zinc silicon phosphide, Copper tin sulfide (CTS), Lanthanum calcium manganite, Copper zinc tin sulfide (CZTS), or Copper zinc antimony sulfide (CZAS).

Non-limiting examples of semiconductor alloy materials that can be used on a working electrode may include the following: Silicon-germanium, Silicontin, Aluminium gallium arsenide, Indium gallium arsenide, Indium gallium phosphide, Aluminium indium arsenide, Aluminium indium antimonide, Gallium arsenide nitride, Gallium arsenide phosphide, Gallium arsenide antimonide, Aluminium gallium nitride, Aluminium gallium phosphide, Indium gallium nitride, Indium arsenide antimonide, Indium gallium antimonide, Cadmium zinc telluride (CZT), Mercury cadmium telluride, Mercury zinc telluride, Mercury zinc selenide, Aluminium gallium indium phosphide, Aluminium gallium arsenide phosphide, Indium gallium arsenide phosphide, Indium gallium arsenide antimonide, Indium arsenide antimonide phosphide, Aluminium indium arsenide phosphide, Aluminium gallium arsenide nitride Indium gallium arsenide nitride, Indium aluminium arsenide nitride, Gallium arsenide antimonide nitride, Copper indium gallium selenide (CIGS), Gallium indium nitride arsenide antimonide, or Gallium indium arsenide antimonide phosphide.

In some preferred embodiments, the plurality of semiconducting nanostructures 122 may comprise ZnO. ZnO is suitable for detecting biomolecules for a wide range of disease biomarkers due to its multifunctional characteristics and ability to form anisotropic nanostructures. The properties of ZnO such as good biocompatibility, wide band gap, non-toxicity, fast electron transfer, high isoelectricpoint (IEP: 9.5), favorable surface for linker chemistry binding, ease in formation of highly c-axis oriented nanostructures at low temperatures (<100° C.) and on various substrates including flexible polymeric substrates, and heightened sensitivity to adsorbed molecules render ZnO an attractive material of choice for affinity sensing applications and with both direct current (DC) and alternating current (AC) electrochemical methods. ZnO is preferred for designing sensors based on electrical transduction. Furthermore, ZnO with its single crystalline state is advantageous in the integration with flexible polymeric substrates, and offers low-cost of ownership manufacturing processes.

It is noted that any semiconducting materials with appropriate functionalization can be utilized on the working electrode(s) of the sensing device. In some embodiments, the metal oxide thin films and nanostructures of ZnO, TiO₂, CNT-TiO₂, SnO₂, ZrO₂, etc. can be used for design of glucose oxide, cholesterol oxidase and other enzymatic sensing devices. For catalytic based sensing devices, the choice of metal/semiconductor (examples: Ag, Au, Pd, Ni, Zn, Co, W, Mo, Mn, and their respective alloys such as ZnO, TiO₂, MnO₂, MoS₂, etc.) as the sensing electrode material may also be dependent on the electrocatalytic properties of the material and the stability of the material at the temperature of operation of the sensor, the pH range of the buffer solution containing the target analytes, and the electrochemical potential window for the detection of the target analytes.

In some embodiments, the plurality of semiconducting nanostructures 122 may be thermally grown on the working electrode in a configuration that aids in radial diffusion of the sample around the plurality of semiconducting nanostructures. As an example, the formation of ZnO nanostructures is described in detail with reference to FIGS. 6A-6C.

A plurality of capture reagents 124 may be attached to the plurality of semiconducting nanostructures 122 on the surface of the working electrode 120. The plurality of capture reagents are configured to selectively bind to one or more target analytes in a fluid sample, thereby effecting changes to electron and ion mobility and charge accumulation in different regions of the semiconducting nanostructures and the fluid sample. The changes to the electron and ion mobility and charge accumulation are detectable with aid of sensing circuitry, and can be used to determine a presence and concentration of the one or more target analytes in the fluid sample.

The capture reagents 124 may include an antibody or antibody fragment, an antigen, an aptamer, a peptide, a small molecule, a ligand, a molecular complex or any combination thereof. Essentially, the capture reagents may be any reagents that have specific binding activity for different target analytes. In some cases, a first capture reagent and a second capture reagent may be antibodies or antibody fragments that specifically bind to epitopes present on a first target analyte and a second target analyte, respectively. Immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. In some cases, the antibody is an antigen-binding antibody fragment such as, for example, a Fab, a F(ab′), a F(ab′)2, a Fd chain, a single-chain Fv (scFv), a single-chain antibody, a disulfide-linked Fv (sdFv), a fragment comprising either a VL or VH domain, or fragments produced by a Fab expression library. Antigen-binding antibody fragments, including single-chain antibodies, can comprise the variable region(s) alone or in combination with the entirety or a portion of the following: hinge region, CH1, CH2, CH3 and CL domains. Also, antigen-binding fragments can comprise any combination of variable region(s) with a hinge region, CH1, CH2, CH3 and CL domains. Antibodies and antibody fragments may be derived from a human, rodent (e.g., mouse and rat), donkey, sheep, rabbit, goat, guinea pig, camelid, horse, or chicken. Various antibodies and antibody fragments may be designed to selectively bind essentially any desired analyte. Methods of generating antibodies and antibody fragments are well known in the art.

The terms “selective” or “specific” binding may be used herein interchangeably. Generally speaking, a ligand that selectively or specifically binds to a target means that the ligand has a high binding affinity for its target, and a low binding affinity for non-target molecules. The dissociation constant (K_{d}) may be used herein to describe the binding affinity of a ligand for a target molecule (e.g., an analyte). The dissociation constant may be defined as the molar concentration at which half of the binding sites of a target molecule are occupied by the ligand. Therefore, the smaller the K_{d}, the tighter the binding of the ligand to the target molecule. In some cases, a ligand has a dissociation constant (K_{d}) for a target molecule of less than 1 mM, less than 100 μM, less than 10 μM, less than 1 μM, less than 100 nM, less than 50 nM, less than 25 nM, less than 10 nM, less than 5 nM, less than 1 nM, less than 500 pM, less than 100 pM, less than 50 pM, or less than 5 pM.

The plurality of semiconducting nanostructures may comprise surfaces that are functionalized with a linking reagent. The capture reagents may be immobilized onto the surfaces of the semiconducting nanostructures via the linking reagent, which is described in detail with reference to FIGS. 7A-7D.

The sensing device is capable of determining the presence and concentration of one or more target analytes in a sample, without the use of any visual markers or labels conjugated to the capture reagents. In various embodiments, the capture detection reagents need not be conjugated or otherwise attached to a detectable label. A detectable label may be a fluorophore, an enzyme, a quencher, an enzyme inhibitor, a radioactive label, one member of a binding pair or any combination thereof. In contrast, other known protein sensing devices often require a label attached to the target protein for detection and quantification. Labeling a biomolecule can drastically change its binding properties, and the yield of the target-label coupling reaction can be highly variable which may affect the detection of protein targets.

The sensing device disclosed herein can circumvent the issues associated with labeling, by using label-free methods for protein detection. Many protein sensors are affinity-based which uses an immobilized capture reagent that binds a target biomolecule. The challenge of detecting a target analyte in solution lies in detecting changes at a localized surface. The use of nanomaterials (e.g. semiconducting nanostructures) as capture surfaces can be particularly beneficial when designing ultra-sensitive electrical sensing devices that rely on measured current and/or voltage to detect binding events. Electrical sensing techniques, such as the modified electrochemical impedance spectroscopy (EIS) technique described herein, have the ability to rapidly detect protein biomarkers at low concentrations. Impedance measurements can be especially useful since they do not require special labels and are therefore suitable for label-free capture operation.

Referring to FIG. 16, the substrate 110 may include a test zone 150 for receiving a sample. The test zone may correspond to a portion or region of the sensing device that is configured to receive or accept a sample. The test zone may be located anywhere on the sensing device, for example at or near an end portion of the substrate. A sample may be applied to the test zone by, e.g., inserting the end portion of the device containing the test zone into a container holding the sample, by pipetting a fluid sample directly onto the test zone, or by holding the test zone of the device under a fluid stream. Generally, the sample is a fluid sample. In other cases, the sample is a solid sample that is modified to form a fluid sample, for example, dissolved or disrupted (e.g., lysed) in a liquid medium.

In some embodiments, a test zone may optionally include a pad or other contact surface. In some cases, the pad may be composed of a woven mesh or a fibrous material such as a cellulose filter, polyesters, or glass fiber. The test zone may further include, without limitation, pH and ionic strength modifiers such as buffer salts (e.g., Tris), viscosity enhancers to modulate flow properties, blocking and resolubilization agents (e.g., proteins (such as albumin), detergents, surfactants (such as Triton X-100, Tween-20), and/or filtering agents (e.g., for whole blood)).

Generally, the sample applied to the test zone 150 may be a fluid sample or a solid sample modified with a liquid medium. In various aspects, the sample is a biological sample. Non-limiting examples of biological samples suitable for use with the immunoassay devices of the disclosure include: whole blood, blood serum, blood plasma, urine, feces, saliva, vaginal secretions, semen, interstitial fluid, mucus, sebum, sweat, tears, crevicular fluid, aqueous humour, vitreous humour, bile, breast milk, cerebrospinal fluid, cerumen, enolymph, perilymph, gastric juice, peritoneal fluid, vomit, and the like. The biological sample can be obtained from a hospital, laboratory, clinical or medical laboratory. In some cases, the immunoassay test using the sensing device is performed by a clinician or laboratory technician. In other cases, the immunoassay test using the sensing device is performed by the subject, for example, at home.

The biological sample can be from a subject, e.g., a plant, fungi, eubacteria, archaebacteria, protist, or animal. The subject can be an organism, either a single-celled or multi-cellular organism. The subject can be cultured cells, which can be primary cells or cells from an established cell line, among others. Examples of cell lines include, but are not limited to, 293-T human kidney cells, A2870 human ovary cells, A431 human epithelium, B35 rat neuroblastoma cells, BHK-21 hamster kidney cells, BR293 human breast cells, CHO Chinese hamster ovary cells, CORL23 human lung cells, HeLa cells, or Jurkat cells. The sample can be isolated initially from a multi-cellular organism in any suitable form. The animal can be a fish, e.g., a zebrafish. The animal can be a mammal. The mammal can be, e.g., a dog, cat, horse, cow, mouse, rat, or pig. The mammal can be a primate, e.g., a human, chimpanzee, orangutan, or gorilla. The human can be a male or female. The sample can be from a human embryo or human fetus. The human can be an infant, child, teenager, adult, or elderly person. The female can be pregnant, suspected of being pregnant, or planning to become pregnant. The female can be ovulating. In some cases, the sample is a single or individual cell from a subject and the biological sample is derived from the single or individual cell. In some cases, the sample is an individual micro-organism, or a population of micro-organisms, or a mixture of micro-organisms and host cells.

In some cases, the biological sample comprises one or more bacterial cells. In some cases, the one or more bacterial cells are pathogens. In some cases, the one or more bacterial cells are infectious. Non-limiting examples of bacterial pathogens that can be detected include Mycobacteria (e.g. M. tuberculosis, M. bovis, M. avium, M. leprae, and M. africanum), rickettsia, mycoplasma, chlamydia, and legionella. Some examples of bacterial infections include, but are not limited to, infections caused by Gram positive bacillus (e.g., Listeria, Bacillus such as Bacillus anthracia, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, Mycobacterium tuberculosis, Mycobacterium avium, Mycobacterium intracellulare, Mycobacterium kansaii, Mycobacterium gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, and Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like.

The biological sample may comprise one or more viruses. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picornavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma—associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses Al-22, 24 (CAl-22 and CA24, CA23 (echovirus 9)), human coxsackieviruses (Bl-6 (CBl-6)), human echoviruses 1-7, 9, 11-27, 29-33, vilyuish virus, simian enteroviruses 1-18 (SEV1-18), porcine enteroviruses 1-11 (PEVI-11), bovine enteroviruses 1-2 (BEV1-2), hepatitis A virus, rhinoviruses, hepatoviruses, cardio viruses, aphthoviruses and echoviruses. The virus may be phage. Examples of phages include, but are not limited to T4, T5, λ phage, T7 phage, G4, P1, Thermoproteus tenax virus 1, M13, MS2, Qβ, ϕX174, Φ29, PZA, Φ15, BS32, B103, M2Y (M2), Nf, GA-1, FWLBc1, FWLBc2, FWLLm3, B4. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotic leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, L P, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O' nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

The biological sample may comprise one or more fungi. Examples of infectious fungal agents include, without limitation Aspergillus, Blastomyces, Coccidioides, Cryptococcus, Histoplasma, Paracoccidioides, Sporothrix, and at least three genera of Zygomycetes. The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agent include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

The biological sample may comprise one or more parasites. Non-limiting examples of parasites include Plasmodium, Leishmania, Babesia, Treponema, Borrelia, Trypanosoma, Toxoplasma gondii, Plasmodium falciparum, P. vivax, P. ovale, P. malariae, Trypanosoma spp., or Legionella spp. In some cases, the parasite is Trichomonas vaginalis.

In some cases, the biological sample is a sample taken from a subject infected with or suspected of being infected with an infectious agent (e.g., bacteria, virus). In some aspects, the biological sample comprises an infectious agent associated with a sexually-transmitted disease (STD) or a sexually-transmitted infection (STI). Non-limiting examples of STDs or STIs and associated infectious agents that may be detected with the devices and methods provided herein may include, Bacterial Vaginosis; Chlamydia (Chlamydia trachomatis); Genital herpes (herpes virus); Gonorrhea (Neisseria gonorrhoeae); Hepatitis B (Hepatitis B virus); Hepatitis C (Hepatitis C virus); Genital Warts, Anal Warts, Cervical Cancer (Human Papillomavirus); Lymphogranuloma venereum (Chlamydia trachomatis); Syphilis (Treponema pallidum); Trichomoniasis (Trichomonas vaginalis); Yeast infection (Candida); and Acquired Immunodeficiency Syndrome (Human Immunodeficiency Virus).

In some cases, the sample can be from an environmental source or an industrial source. Examples of environmental sources include, but are not limited to, agricultural fields, lakes, rivers, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, and pharmaceutical compounding centers. The sample can be a forensic sample (e.g., hair, blood, semen, saliva, etc.). The sample can comprise an agent used in a bioterrorist attack (e.g., influenza, anthrax, smallpox).

In some cases, more than one sample can be obtained from a subject or source, and multiple immunoassay tests using a single sensing device or apparatus described herein can be performed. In some cases, 2, 3, 4, 5, 6, 7, 8, 9, 10, 15, 20, 25, 30, 35, 40, 45, 50 or more samples can be obtained. In some cases, more than one sample may be obtained over a period of time, for example, to monitor disease progression or to monitor a biological state or condition (e.g., cardiac conditions). Generally, the sensing devices of the disclosure are configured for repeated or continuous use. Alternatively, the sensing devices can be one-time use (e.g., disposable).

In some cases, the subject is affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample can be from a subject who has a specific disease, disorder, or condition, or is suspected of having (or at risk of having) a specific disease, disorder or condition. For example, the biological sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample can be from the cancer and/or normal tissue from the cancer patient. In some cases, the sample is a biopsy of a tumor.

The biological sample can be processed to render it competent for performing any of the methods using any of the devices or kits provided herein. For example, a solid sample may be dissolved in a liquid medium or otherwise prepared as a liquid sample to facilitate flow along the test strip of the device. In such cases where biological cells or particles are used, the biological cells or particles may be lysed or otherwise disrupted such that the contents of the cells or particles are released into a liquid medium. Molecules contained in cell membranes and/or cell walls may also be released into the liquid medium in such cases. A liquid medium may include water, saline, cell-culture medium, or any solution and may contain any number of salts, surfactants, buffers, reducing agents, denaturants, preservatives, and the like.

Generally, the sample contains or is suspected of containing one or more target analytes. In various aspects, the sample may contain at least a first analyte and a second analyte. The term “analyte” as used herein may refer to any substance that is to be analyzed using the methods and devices provided herein. The immunoassay sensing devices and arrays disclosed herein may be configured to simultaneously detect the presence of 1, 2, 3, 4, 5, 6, 7, 8, 9, 10 or more analytes in a sample. The immunoassay sensing devices and arrays disclosed herein can be capable of simultaneous and multiplexed detection of multiple target analytes in a single sample.

Non-limiting examples of analytes may include proteins, haptens, immunoglobulins, hormones, polynucleotides, steroids, drugs, infectious disease agents (e.g., of bacterial or viral origin), drugs of abuse, environmental agents, biological markers, and the like. In one case, the immunoassay detects at least a first analyte, wherein the first analyte is luteinizing hormone (LH). In another case, the immunoassay detects at least a first analyte, wherein the first analyte is human chorionic gonadotropin (hCG). In another case, the immunoassay detects at least a first analyte and a second analyte, wherein the first analyte is estrone-3-glucoronide (E3G) and the second analyte is luteinizing hormone (LH). In another case, the immunoassay detects at least a first analyte and a second analyte, wherein the first analyte is a surface antigen on a first viral particle (e.g., Influenza A) and the second analyte is a surface antigen on a second viral particle (e.g., Influenza B). In another case, the immunoassay detects at least a first analyte, wherein the first analyte is 25-hydroxyvitamin D, 25-hydroxyvitamin D2 [25(OH)D₂], or 25-hydroxyvitamin D3 [25(OH)D₃]. In another case, the immunoassay detects at least a first analyte and a second analyte, wherein the first analyte is triiodothyronine (T3) and the second analyte is thyroxine (T4). In another case, the immunoassay detects at least a first analyte, wherein the first analyte is an allergen. Non-limiting examples of allergens may include: Balsam of Peru, fruit, rice, garlic, oats, meat, milk, peanuts, fish, shellfish, soy, tree nuts, wheat, hot peppers, gluten, eggs, tartrazine, sulfites, tetracycline, phenytoin, carbamazepine, penicillin, cephalosporins, sulfonamides, non-steroidal anti-inflammatories (e.g., cromolyn sodium, nedocromil sodium, etc.), intravenous contrast dye, local anesthetics, pollen, cat allergens, dog allergens, insect stings, mold, perfume, cosmetics, semen, latex, water, house dust mites, nickel, gold, chromium, cobalt chloride, formaldehyde, photographic developers, fungicide, dimethylaminopropylamine, paraphenylenediamine, glyceryl monothioglycolate, toluenesulfonomide formaldehyde.

The sensing device may be used to test for the presence or absence of at least a first analyte and a second analyte in a sample. In some cases, the sensing device may be used to determine an amount or a relative amount of at least a first and second analyte in a sample.

The presence or absence of analytes may be indicative of a disease or disorder in a subject. The presence or absence of analytes may be indicative of a biological state or condition of a subject. In some cases, the presence or absence of analytes indicates that a subject has or is at risk of developing a disease. In some cases, the presence or absence of analytes indicates that a subject has a disorder (e.g., thyroid disorder). In some cases, the presence or absence of analytes indicates that a subject has a deficiency (e.g., vitamin deficiency). In some cases, the presence or absence of analytes indicates that a product (e.g., a food or drink product) contains an allergen.

The sensing device 100 may be an electrochemical sensing device configured for both catalytic and affinity-based detection of one or more target analytes in a sample. A catalytic sensor(s) or catalytic sensing utilizes molecules (such as enzymes) that catalyze a biochemical reaction on the sensing surface with the target molecule and detection based on the resulting products. An affinity-based sensor(s) or affinity-based sensing is designed to monitor binding of the target molecule and uses other specific binding molecules (e.g., proteins, lectins, receptors, nucleic acids, whole cells, aptamers, DNA/RNA, antibodies or antibody-related substances, etc.) for biomolecular recognition.

In many embodiments, the sensing devices or arrays disclosed herein can be configured to simultaneously detect and quantitate different isoforms of a single protein. The molecules associated with the catalysis-based reaction may be anchored onto the sensing surface (e.g. working electrode) through an affinity-based mechanism to ensure that the chemical reaction(s) occurs in proximity of the sensing surface for enhanced sensitivity of detection. The output electrical signals for both catalytic and affinity sensors/sensing is measured in current, voltage, and impedance.

Amperometric (i.e. DC current—DC voltage—time) and impedimetric sensors are electroanalytical methods for characterization of the surface phenomena and changes at the sensing electrode surfaces. Amperometric sensors can measure changes to electric current resulting from either catalytic mechanisms and/or affinity binding mechanisms occurring at the sensing electrode surfaces under an applied field/potential and that are related to the concentration of the target species or analytes present in the solution. Voltammetry and chronoamperometry are subclasses of amperometry. In voltammetry, current is measured by varying the potential applied to the sensing electrode. In chronoamperometry, current is measured at a fixed potential, at different times after the start of sensing.

The aforementioned sensors and sensing methods are particularly well-suited for detection of catalytic processes and their associated effects modulated due to kinetic and thermodynamic properties. Signal transduction and quantification occurs through the dynamic transfer of electrons resulting from the catalytic processes and/or the associated chemical reactions to the sensing electrode surface. Specificity in detection of target species or analytes can be achieved through the choice of the catalytic processes and the higher reaction rate kinetics occurring within the electrochemical potential window, which can result in amplified signals through the sensing electrode surface.

Impedimetric sensors are well-suited for detection of binding events on the sensing electrode surface. Analytes can interact with the sensing electrode through selective treatments applied to the electrode surface in the form of cross-linkers (e.g., antibodies, nucleic acids, ligands, etc.) that are covalently conjugated onto sensing electrode surface. The impedance Z of the sensor can be determined by applying a voltage perturbation with a small amplitude and detecting the current response. The impedance Z is the quotient of the voltage-time function V(t) and the resulting current-time function I(t), and given as follows:

$$Z = \frac{V ( t )}{I ( t )} = \frac{{V}_{0} sin ( 2 II f t )}{{I}_{0} sin ( 2 IIf t + ϕ )} = \frac{1}{Y}$$

where V₀ and I₀ are the maximum voltage and current signals, f is the frequency, t the time, ϕ the phase shift between the voltage-time and current-time functions, and Y is the complex conductance or admittance. The measured impedance associated with biomolecule binding is a complex value, since the current can differ in terms of not only the amplitude but also it can show a phase shift ϕ compared to the voltage-time function. Thus, the value can be described either by the modulus |Z| and the phase shift ϕ or alternatively by the real part ZR and the imaginary part ZI of the impedance. Therefore, the results of an impedance measurement can be illustrated in two different ways: using a Bode plot, which plots log |Z| and ϕ as a function of log f, or using a Nyquist plot, which plots ZR and ZI. Both of these plots can be used to establish calibration responses of the sensing device towards real-time detection and quantification of the target species or analytes. Sensitivity and specificity in detection can be achieved through deconstruction of the Nyquist and Bode plots, by identifying the frequency range where the electrical double layer effects due to the binding events of the target species occur and quantifying the change in impedance with concentration within this range.

In various embodiments, when a working electrode comprising ZnO nanostructures is exposed to a sample (e.g., an ionic solution comprising biomolecules), a potential difference is generated at the electrode/electrolyte interface due to the unequal distribution of charges. As a consequence of biomolecular binding events at the surface of the ZnO nanostructures, redistribution of charges in the working electrode and ions in the electrolyte can result in formation of a space-charge region within the ZnO nanostructures and an electrical double layer at the interface between the electrode and the electrolyte. Evaluation and quantification of biomarker binding can be achieved by measuring the changes in electrode resistance or capacitance at selected frequencies.

The changes to space-charge capacitance and overall impedance at the ZnO nanostructures/electrolyte interfaces can be characterized by respectively using a direct current (DC)-based Mott-Schottky technique and an alternating current (AC)-based electrochemical impedance spectroscopy (EIS) technique towards detection of target analytes or biomarkers. Correlation in output signal response with concentration can be established between the DC and AC electrochemical detection techniques.

As previously described, the plurality of capture reagents of the sensing device are configured to selectively bind to one or more target analytes in a sample, thereby effecting changes to electron and ion mobility and charge accumulation in different regions of the semiconducting nanostructures and the sample. The changes to the electron and ion mobility and charge accumulation can be detected with aid of sensing circuitry, and can be used to determine a presence and concentration of the one or more target analytes in the sample. The changes to the electron and ion mobility and charge accumulation can be transduced into electrical impedance and capacitance signals. The signals may be indicative of interfacial charge modulation comprising of the changes to the electron and ion mobility. Additionally, the signals may be indicative of capacitance changes to a space-charge region formed in the semiconducting nanostructures upon binding of the one or more target analytes to the capture reagents. The changes may comprise simultaneous modulation to the ion mobility in one or more regions adjacent or proximal to the semiconducting nanostructures.

The sensing circuitry may comprise hardware, software, or a combination of software and hardware. The sensing circuitry may comprise a single or multiple microprocessors, field programmable gate arrays (FPGAs), or digital signal processors (DSPs). The sensing circuitry may be electrically connected to the sensing device. In some embodiments, the sensing circuitry may be part of the sensing device, for example the sensing circuitry may be assembled or disposed on the substrate. Alternatively, the sensing circuitry may be remote to the sensing device.

The sensing circuitry can be configured to implement a plurality of electrochemical detection techniques for detecting the capacitance changes and impedance changes. The plurality of electrochemical detection techniques may comprise, for example (1) a modified Electrochemical Impedance Spectroscopy (EIS) technique for measuring the impedance changes and (2) Mott-Schottky technique for measuring the capacitance changes. The modified EIS technique is capable of distinguishing the electrical impedance signals from background noise at low concentrations of the target analytes in the sample. The sensing circuitry can be configured to analyze the electrical impedance and capacitance signals by concurrently analyzing a set of Nyquist plots obtained via the modified EIS technique and a set of Mott-Schottky plots obtained via the Mott-Schottky technique. The modified EIS technique may comprise (1) sectioning an interfacial charge layer into a plurality of spatial dielectric z-planes along a direction orthogonal to the interface between the fluid sample and the semiconducting nanostructures, and (2) probing each of the plurality of z-planes with a specific frequency selected from a range of frequencies. Specific binding of different target analytes to the capture reagents may occur at known spatial heights within the interfacial charge layer. Accordingly, the sensing circuitry can be configured to determine the presence and concentration of each of the different target analytes by measuring the capacitance and impedance changes at specific frequencies corresponding to their respective z-planes at the known spatial heights within the interfacial charge layer.

The inherent non-stoichiometric nature of ZnO may result in generation of oxygen vacancies, and the ease in forming surface bonds with hydroxyl molecules and other ions can render the ZnO surface sensitive to the pH of the biofluids and environment. Thus, ZnO-based sensing devices may develop drifts in signal output over time, independent of detection modality, especially when exposed to varying pH solutions in the presence of enzymatic reactions that involve generation of hydrogen peroxide. In addition, protein biomolecules can easily denature when exposed to temperature, environment, and pH outside the established range of their stability.

To mitigate the above effects, a sample may be provided in a room temperature ionic liquid (RTIL) electrolyte buffer in some embodiments. The stability and reliability of the bound proteins to the functionalized nanostructured ZnO surfaces can be improved with the use of RTIL as the electrolyte solvent buffer containing the specific protein antibodies, and that can conjugate with the functionalized ZnO surface during the immunoassay steps. The RTIL can also provide stability of the bound proteins during subsequent storage and handling and from exposure to environment. In simple electrolyte solvent solutions, the protein charge is typically determined by the equilibrium protonation of hydroxyl- and amino-groups, and depends on the pH of the environment, whose variations can even reverse the sign of the overall charge. In contrast, for RTILs, dispersion energy, ion size, and additional H-bonding sites can be useful in determining protein characteristics. Unlike molecular solvents that are charge neutral, RTILs are molten salts at room temperature composed solely of polyatomic cations and anions.

The properties of RTILs can be changed according to the requirement by modifying their constituents (cation and anion). Although they can stabilize the protein over a wide range of temperature, the thermal stability of proteins depends on the appropriate choice of RTILs as proteins are not homogeneously stable in all type of RTILs. In some cases, the stability and activity of proteins is affected by many factors such as polarity, hydrophilicity vs. hydrophobicity and hydrogen-bond capacity of RTILs, excipients, and impurities. RTILs containing chaotropic (large-sized and low charged, weakly hydrated ions that decrease the structure of water) cations and kosmotropic (small-sized and high charged, strongly hydrated ions that increase the structure of water) anions can optimally stabilize the biological macromolecules. In some embodiments, the kosmotropicity order of anions and cations can be determined by using viscosity B-coefficients and other parameters such as hydration entropies, hydration volumes, heat capacity, NMR B-coefficients and ion mobility.

In one embodiment, RTILs containing chaotropic cations and kosmotropic anions can be selected to independently and optimally stabilize the target proteins chosen i.e. cTnl and/or cTnT, NT-proBNP, and CRP. Intermixing of protein biomolecules and ensuring cross-reactivity response is well below the noise threshold in signal transduction response from each of the bound antibodies in the detection of their specific target proteins can be achieved.

In some embodiments, the plurality of semiconducting nanostructures may be disposed on two or more electrodes comprising of a first electrode and a second electrode. A first capture reagent may be attached to the semiconducting nanostructures on the first electrode and configured to selectively bind to a first target analyte. A second capture reagent may be attached to the semiconducting nanostructures on the second electrode and configured to selectively bind to a second target analyte. The sensing device is capable of simultaneously determining the presence and concentrations of the first and second target analytes upon binding of the target analytes to the respective capture reagents.

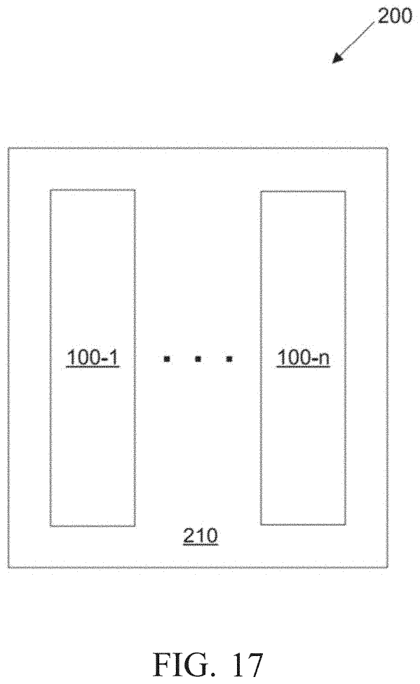

In some embodiments, the first electrode may be part of a first sensing device, and the second electrode may be part of a second sensing device. The first and second sensing devices may be provided on a common sensing platform. For example, FIG. 17 shows a sensing array 200 comprising a plurality of sensing devices 100 for detecting a plurality of different target analytes in a fluid sample. The array may comprise two or more sensing devices (e.g., 100-1 through 100-n, where n can be any integer greater than two) disposed on a common substrate 210. Alternatively, the sensing devices may be provided separately and then assembled onto the substrate 210. The sensing devices may each comprise a working electrode having a plurality of semiconducting nanostructures disposed thereon and a capture reagent attached to the semiconducting nanostructures. The sensing devices may or may not have the same type of semiconducting nanostructures or materials. The sensing devices may comprise different capture reagents that are configured to selectively bind to the different target analytes in the fluid sample. The selective binding is configured to effect changes to electron and ion mobility and charge accumulation in different regions of the semiconducting nanostructures and the fluid sample. Each of the sensing devices can be configured to determine a presence and concentration of a different target analyte in the fluid sample based on detected changes to the electron and ion mobility and charge accumulation.

A method of detecting a plurality of different target analytes in a fluid sample may include providing the sensing array described herein, and applying the fluid sample containing one or more target analytes to the sensing array. The method may include using each of the sensing devices to determine the presence and concentration of a different target analyte in the fluid sample, based on the detected changes to the electron and ion mobility and charge accumulation in the different regions of the semiconducting nanostructures and the fluid sample.

In some embodiments, an array 200 may comprise a first sensing device 100-1 and a second sensing device 100-2 capable of simultaneously determining the presence and concentrations of first and second target analytes upon binding of the target analytes to the respective capture reagents. In some embodiments, the first and second target analytes may comprise different isoforms of a same type of biomarker. In some embodiments, the target analytes may comprise a plurality of cardiac biomarkers, and the plurality of capture reagents may comprise a plurality of antibodies that are specific to the plurality of cardiac biomarkers.

As noted previously, there is a need for the rapid, quantitative, specific, and multiplex detection and measurement of target analyte concentrations at point of care. The ability to perform multiplexed detection can provide significant advantages for point of care diagnostics in that it allows for the simultaneous monitoring of multiple markers in a single sample. The multiplexing can support the performance of both negative and positive controls in the same sample. Together, these attributes can significantly improve the specificity and sensitivity with which certain diseases and physiological conditions can be detected and diagnosed.

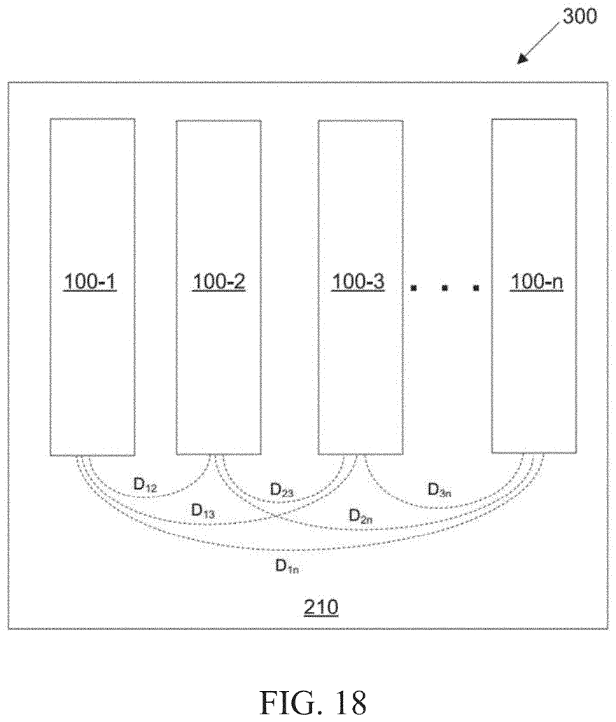

The array 200 shown in FIG. 17 is capable of simultaneous and multiplexed detection of different target analytes present in a fluid sample using a plurality of electrochemical detection techniques. FIG. 18 shows a multi-configurable sensing array 300 comprising a plurality of sensing devices 100-1, 100-2, 100-3 through 100-n. The electrodes of the sensing devices can be connected to sensing circuitry configured for simultaneous acquisition and multiplexing of electrical signals from the sensing devices. The sensing devices can be configured for both catalytic and affinity-based sensing. A working electrode in each sensing device can be independently functionalized for specific detection of a target analyte which may be a biomarker. Different sensing devices in the array 300 may comprise different capture reagents that are configured to selectively bind to the different target analytes in the fluid sample. The output from each sensing device may be independently measured and transduced (e.g., amperometric or impedometric) to provide a combinatorial/multiplexed result relating to the end physiological state being predicted. For example, D₁₂ may be the multiplexed result between sensing devices 100-1 and 1002; D₂₃ may be the multiplexed result between sensing devices 100-2 and 1003; D₁₃ may be the multiplexed result between sensing devices 100-1 and 1003; D₁ₙ may be the multiplexed result between sensing devices 100-1 and 100-n, and so forth. In some embodiments, the output from more than two sensing devices, or all of the sensing devices, may be independently measured and transduced (e.g., amperometric or impedometric) to provide a combinatorial/multiplexed result relating to the end physiological state being predicted. For example, D_{123 . . . n} may be the multiplexed result between sensing devices 100-1, 100-2, 100-3 through 100-n. Any number or combination of multiplexed results from the sensing devices may be contemplated. The output from the two or more sensing devices can be weighed the same (e.g. each output accorded a same weight) or weighed differentially (e.g. different outputs accorded different weights). In some embodiments, the output from a sensing device may be compared or correlated with the output(s) of one or more other sensing devices. For example, the output from sensing device 100-1 may be compared or correlated with the output(s) of one or more other sensing devices (e.g., 100-2, 100-3) to improve specificity and sensitivity in detecting and diagnosing certain diseases and physiological conditions.

The multi-configurable array 300 can be configured for detection of multiple analytes that may be useful in disease detection. In some embodiments, the array can be used for paired and simultaneous detection of disease markers in body fluids in a non-invasive manner such as: (a) Inflammatory marker, interleukin-6 (IL-6) and diabetes marker, Glucose in human sweat; and/or (b) Inflammatory markers, interleukin-6 (IL-6) and C-reactive protein (CRP) and muscular dystrophy markers, creatine kinase (CK-MB) in finger pricked capillary blood. In some embodiments, the array can be integrated with other sensors within wearable fabric, devices, and medical instruments such as strips, catheters, probes, patches for non-communicable disease diagnosis such as cardiac, cancer, Alzheimer's, muscular dystrophy, inflammatory markers, etc.

The array 300 may be capable of supporting simultaneous detection of multiple target analytes in a single sample volume. The volume may be 150 μL, 140 μL, 130 μL, 120 μL, 110 μL, 100 μL, 90 μL, 80 μL, 70 μL, 60 μL, 50 μL, 40 μL, 30 μL, 20 μL, 10 μL, 1 μL, or any value therebetween. In some embodiments, the array 300 may be capable of supporting simultaneous detection of multiple target analytes in a single, submilliliter sample volume (e.g. <30 μL). In some embodiments, simultaneous and multiplexed detection of the target analytes can be completed in a short time (e.g., on the order of a few minutes or less), and using <20 μL of sample volume. In some embodiments, simultaneous and multiplexed detection of the target analytes can be achieved using about 10-20 μL of sample volume.

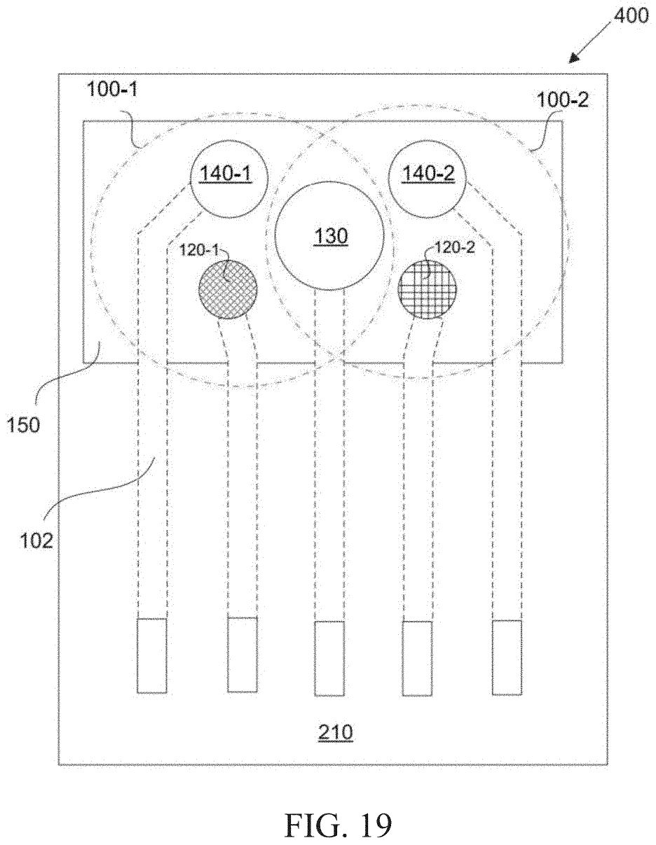

FIG. 19 shows an array 400 comprising a first sensing device 100-1 and a second device 100-2 in accordance with some embodiments. The first and second sensing devices may be similar to the sensing devices described elsewhere herein. In the example of FIG. 19, the first and second sensing devices may share a common reference electrode (RE) 130, instead of each sensing device having its own reference electrode. The common reference electrode can provide a stable and known electrode potential to the electrochemical cell comprising of the first and second sensing devices. The first and second sensing devices can operate based on the same reference electrode potential, thereby permitting simultaneous and multiplexed detection of target analytes, and calibration of results between the two sensing devices.

The first sensing device 100-1 may comprise a working electrode (WE) 120-1 and a counter electrode (CE) 140-1. The second sensing device 100-2 may comprise a working electrode (WE) 120-2 and a counter electrode (CE) 140-2. The common RE 130 may be disposed between the working electrodes of the two sensing devices. The common RE 130 may also be disposed between the counter electrodes of the two sensing devices. The WE 120-1, RE 130, and CE 140-1 may be located in proximity to each other in a first region of the substrate 210. The WE 120-2, RE 130, and CE 140-2 may be located in proximity to each other in a second region of the substrate 210. The first and second regions may be part of a test zone 150. The first sensing device may comprise a first capture reagent configured to selectively bind to a first target analyte. The second sensing device may comprise a second capture reagent configured to selectively bind to a second target analyte. In some embodiments, the common RE 130 may have a larger surface area than each of the working electrodes and counter electrodes. For example, the surface areas of WE:CE:RE may be designed in the ratio of 1:1:4 to ensure sufficient output signal response due to binding events at the working electrodes.

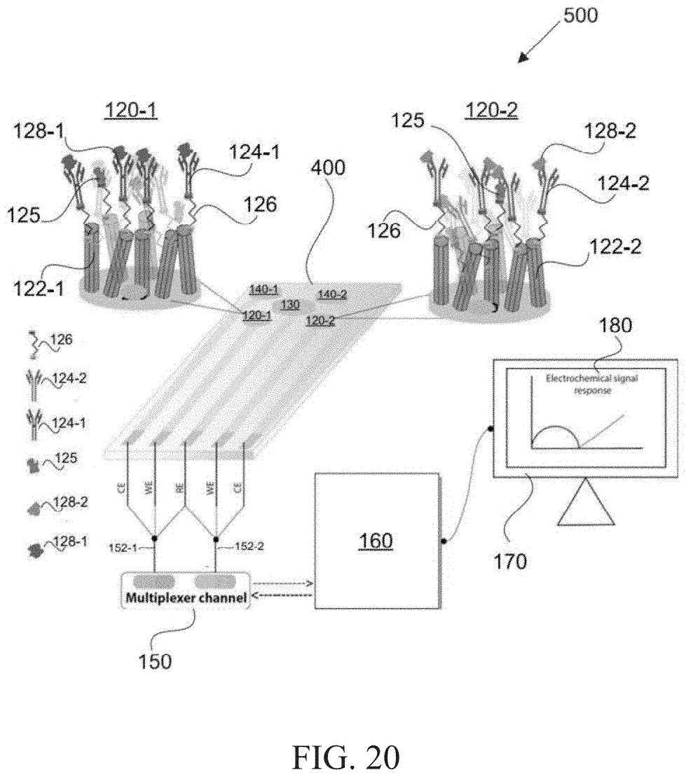

FIG. 20 shows a sensing system 500 in accordance with some embodiments. The system 500 may comprise a multi-configurable array of sensing devices, for example array 400 described with reference to FIG. 19. The array 400 may comprise a first sensing device and a second sensing device as described elsewhere herein. The first sensing device may include a first working electrode (WE) 120-1 and a first counter electrode (CE) 140-1. The second sensing device may include a second working electrode (WE) 120-2 and a second counter electrode (CE) 140-1. The first and second sensing devices may share a common reference electrode (RE) 130.

FIG. 20 further shows a magnified schematic view of the functionalized working electrode (WE) 120 of each sensing device. As previously described, each working electrode can be independently functionalized for specific detection of a target biomarker(s). The output from each sensing device can be independently measured and transduced (e.g., amperometric or impedometric) to provide a multiplexed outcome relating to the end physiological state being predicted.

Referring to FIG. 20, a plurality of semiconducting nanostructures 122 may be disposed on the WEs 120. For example, first semiconducting nanostructures 122-1 may be disposed on the surface of the first WE 120-1, and second semiconducting nanostructures 122-2 may be disposed on the surface of the second WE 120-2. In some embodiments, the first and second semiconducting nanostructures may be formed of a same semiconductor or semiconductor alloy material. Alternatively, the first and second semiconducting nanostructures may be formed of different types of semiconductor or semiconductor alloy material. In some instances, each of the first and second semiconducting nanostructures may comprise two or more types of semiconductor or semiconductor alloy material. The semiconducting nanostructures can be grown or deposited on the surface of the working electrodes. In some embodiments, the first and second semiconducting nanostructures may comprise ZnO nanostructures, as described in more detail with reference to FIGS. 6A-C.

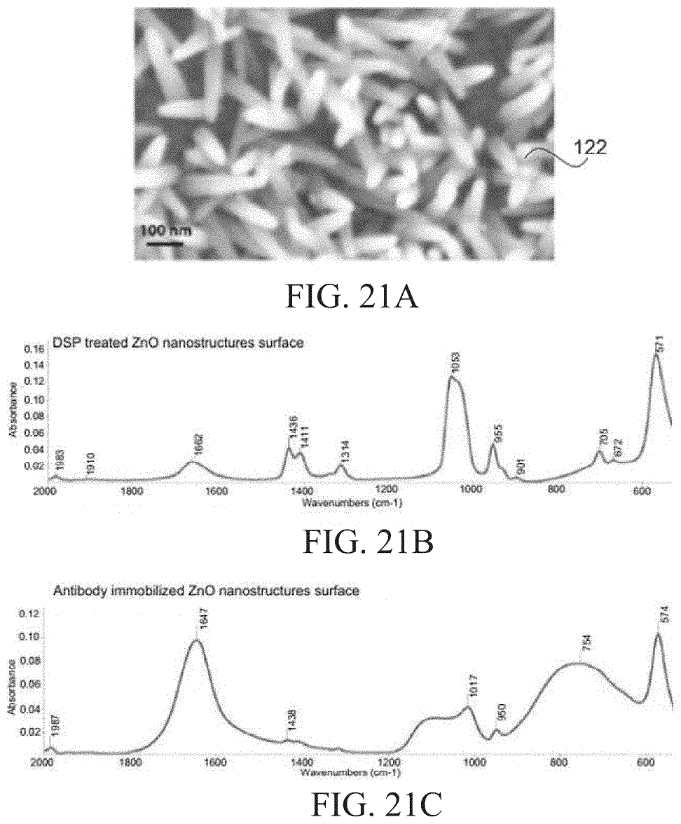

FIG. 21A shows an SEM micrograph of ZnO nanostructures that are selectively grown on the working electrodes of the sensing array using low temperature aqueous hydrothermal growth mechanism. The nanostructures may be elongated, and may include nanorods or nanopillars. In some embodiments, the nanostructures may have an aspect ratio of about 1:4. The nanostructures may be formed having different shapes, sizes, dimensions, and/or aspect ratios depending on the growth conditions. In some embodiments, the ZnO nanostructures may be grown by tuning the chemical reactions between the precursors Zn(NO3)2.6H2O and HMTA dissolved in water. The thermal decomposition and hydrolysis reactions of these precursors results in the formation of zinc hydroxyl species which upon dehydration form ZnO nuclei. Pre-seeded regions on the working electrodes can then act as nucleation sites for the aligned growth of ZnO nanostructures. The higher surface energy difference between polar and non-polar planes derives faster growth of ZnO along polar planes resulting in c-axis oriented crystalline growth of wurtzite ZnO nanostructures. The SEM micrograph in FIG. 21A shows the morphology of synthesized ZnO nanostructures as vertically grown hexagonal shaped rod-like structures and uniform growth on the working electrodes. The SEM characterization indicates uniform growth of hexagonal shaped ZnO nanostructures at the pre-seeded working electrodes. The as-synthesized ZnO nanostructures can be used to aid detection of various target analytes (e.g. cardiac biomarkers) using the sensing array of FIGS. 4 and 5 as described elsewhere herein.

FIG. 21B is an ATR-FTIR spectra showing evidence of DSP functionalization on nanostructured ZnO sensing surface in the range between 2000 cm⁻¹ and 500 cm⁻¹. FIG. 21C is an ATR-FTIR spectra showing evidence of antibody immobilization on nanostructured ZnO sensing surface in the range between 2000 cm⁻¹ and 500 cm⁻¹. Referring to FIG. 21B, functionalization of ZnO nanostructures with linking reagent (e.g. thiol-based DSP linker molecules) can provide binding sites for immobilization of the capture reagent (e.g. antibodies). The peak at 571 cm⁻¹ is associated with the ZnO nanostructures and is stable as the immunoassay is being conducted on the sensing array. The peaks observed at 1053 cm⁻¹ and 1314 cm⁻¹ are assigned to stretching vibrations of v(C-O) and v(N-O) respectively. The spectral features v(C-O) is characteristic of the ester linkage and v(N-O) represents the symmetric stretch of nitro groups both of which disappears with immobilization of the antibody molecule. The other succinimidyl identifier groups that show evidence of DSP binding to ZnO surfaces are the carbonyl stretch in primary amides (v(C═O)) at 1662 cm⁻¹ and bending vibrations of alkane stretch (v(C—H)) with two peaks at 2915 cm⁻¹ and 3000 cm⁻¹ (not shown). Bands assigned at 1411 cm⁻¹ and 1436 cm⁻¹ are characteristic of methylene scissors deformation in the bound DSP molecule. Referring to FIG. 21C, appearance of broad band between 1200 cm⁻¹ and 1020 cm⁻¹ in the spectra is characteristic of v(C—C, C—N) and confirms aminolysis of NHS groups in DSP with primary amines in antibody establishing a stable conjugation of the antibody to the linker functionalized ZnO nanostructure surfaces grown on Au working electrodes.

The ATR-FTIR spectral of the surface functionalized ZnO nanostructures (shown in FIGS. 6B and 6C) can be obtained using an FTIR spectrometer equipped with a deuterated, L-alanine doped triglycine sulfate (DLaTGS) Detector with KBr window and validation motor. The spectrometer can be fitted with a sampling stage equipped with a 60° diamond ATR crystal and the sample can be held with a swivel clamp that applied an even and constant force during the acquisition of the spectra. Each FT-IR spectrum collected on the sample represents the average of 200 scans at 4 cm⁻¹ resolution in the scan range of 4000-400 cm⁻¹.

The samples for FTIR analysis can be prepared as follows: (1) deposit a thin layer of gold (dimensions) on the glass slides followed by ZnO seed deposition; (2) clean the glass slides subsequently in acetone, isopropyl alcohol and deionized water prior to use; (3) grow the ZnO nanostructures on seeded substrates and wash with DI water to remove growth residues; (4) treat the nanostructured ZnO substrates with 10 mM DSP in DMSO for an hour; (5) after DSP functionalization, rinse the substrates with DMSO to remove unbound molecules and stored with silica desiccants for analysis. Some of the samples are washed α-cTnl antibody. After 30 minutes, the antibody treated substrates are washed with PBS and the FTIR analysis is then performed.

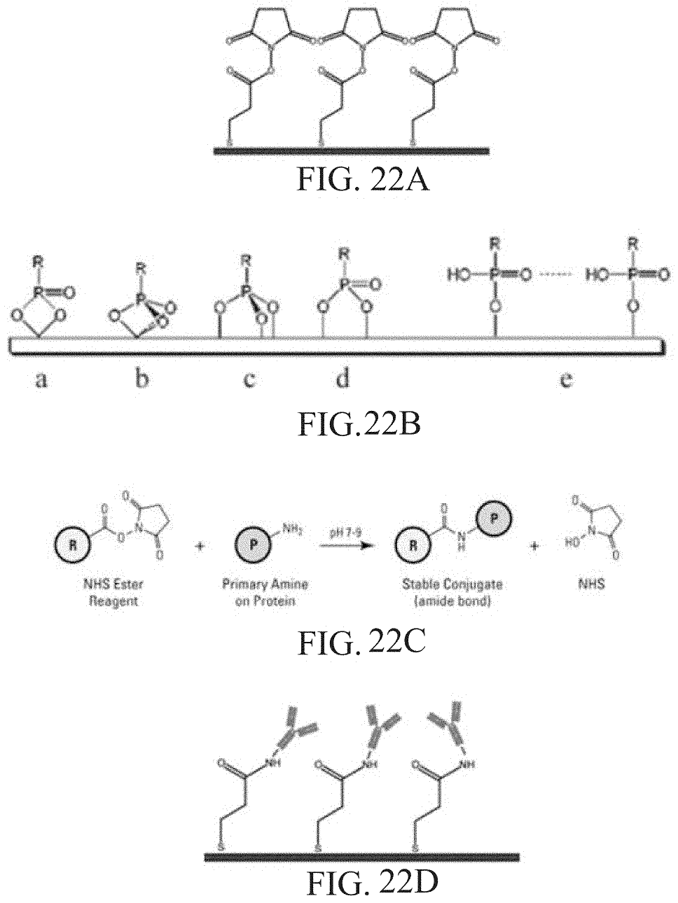

Referring back to FIG. 20, a plurality of capture reagents 124 may be directly or indirectly attached to the plurality of semiconducting nanostructures 122. In some embodiments, a sample comprising the target analytes 128 may be provided with a blocking buffer. The blocking buffer may comprise a protein 125 that can block or cap the binding sites of excess linking reagents that did not bind to a capture reagent. The blocking buffer can improve the signal-to-noise ratio of the sensing device. As shown in FIG. 20, a first capture reagent 124-1 may be attached to the first semiconducting nanostructures 122-1 on the first electrode 120-1, and configured to selectively bind to a first target analyte 128-1. A second capture reagent 124-2 may be attached to the second semiconducting nanostructures 122-2 on the second electrode 120-2, and configured to selectively bind to a second target analyte 128-2. In some embodiments, the semiconducting nanostructures 122-1 and 122-2 may be functionalized with a linking reagent 126, and the capture reagents 124-1 and 124-2 may be immobilized onto the semiconducting nanostructures 122-1 and 122-2 via the linking reagent 126, as described in more detail with reference to FIGS. 22A-C.

In some embodiments, a working electrode may preferably include a Au surface which offers ease of functionalization with organic linker molecules with thiol, carboxylic, etc. terminal ends. The terminal ends of the organic linker molecules bind to the Au surface through adsorption processes and are thermodynamically stable. In some embodiments, the WE may have an immersion Au surface finish which has energetically favored sites for binding of the terminal ends of the organic linker molecules in comparison to other types of thin film Au deposition methods (example: evaporation, sputtering, etc.). In other embodiments, the WE may have an immersion Ag surface, except the Ag surface tends to oxidize more easily than Au surface. A sensing WE with semiconducting ZnO, TiO₂, or MoS₂ layers can be functionalized with selective linker chemistry that subsequently conjugate with capture reagents (e.g. biomolecules, small organic molecules, etc.) required for target analyte recognition. In some embodiments, a sensing WE with semiconducting ZnO, TiO₂, or MoS₂ layers can be functionalized with non-biological chemical capture reagents, for example for the detection of certain chemicals or chemical compounds in the sample.

The selection of linker molecules can be influenced by several factors including bond-stability, position of functional groups, pH, presence/absence of amine groups for interaction with antibody, surface charge etc. The availability of different functional groups in linker molecules can enable the immobilization of antibody through stable covalent linkage, and the antibody-antigen interactions provide specificity for detection of target analytes. In the embodiments described herein, binding of capture reagents and subsequent biomolecules to the affinity immunoassay leads to changes in the ion diffusion profile near the nanostructures and hence changes in electrical properties (capacitance, resistance, etc.). The electrochemical detection methods described herein include means to directly characterize the capture reagent—target analyte interactions based on charge perturbations at the electrode-electrolyte interface. In some embodiments, functionalization may include the use of thiol and phosphonic acid terminated groups on ZnO nanostructures or thin films.

FIG. 22A shows the functionalization of a sensing WE using the linker molecule dithiobis(succinimidyl propionate) (DSP) in accordance with an embodiment. The DSP contains an amine-reactive N-hydroxysuccinimide (NHS) ester at each end of an 8-carbon spacer arm containing a cleavable disulfide bond. The DSP reacts with the Au surface to form stable Au-thiol bonds from which the amine-reactive NHS ester extend. The NHS esters react with primary amines at pH 7-9 to form stable amide bonds, along with release of the N-hydroxy-succinimide leaving group. Proteins, including antibodies, generally have several primary amines in the side chain of lysine (K) residues and the N-terminus of each polypeptide that are available as targets for NHS-ester crosslinking reagents. FIG. 22B shows the functionalization of a sensing WE using phosphoric based organic linker molecules in accordance with another embodiment, that can form stable Au-phoshonic bonds represented by bond configurations a-e. Capture reagents (e.g., biomolecules) can include proteins, small molecules, antibodies, nucleic acids, etc., and can be customized for the binding and detection of specific target analytes of interest. The process of immobilizing the capture reagents on the functionalized sensing WE surfaces and the subsequent detection of biomarkers may be described as an assay. FIG. 22C shows a schematic reaction for amine-reactive NHS ester reagents with primary amines on a protein at pH 7-9 to form stable amide bonds, along with release of the N-hydroxy-succinimide leaving group. Proteins, including antibodies, generally have several primary amines in the side chain of lysine (K) residues and the N-terminus of each polypeptide that are available as targets for NHS-ester crosslinking reagents. FIG. 22D illustrates a DSP functionalized sensing WE surface forming stable amide bonds with the primary amine groups of a selected antibody of interest.

Accordingly, the multi-configurable sensing array described herein may comprise sensing working electrodes that can be independently functionalized with the appropriate linker chemistry and different capture reagents that are specific to the detection of different target analytes. Affinity-based sensors/sensing can leverage the above functionalization strategies. In catalytic-based sensors/sensing, binding of catalysts to the electrode surfaces can ensure that the chemical reaction and electron transfer occur in proximity to the electrode surfaces.

Referring back to FIG. 20, the sensing system 500 may further comprise a multiplexer 150, sensing circuitry 160, and computing device 170. The array 400 may be electrically connected to the multiplexer 150 and the sensing circuitry 160. The multiplexer may comprise a plurality of channels 152 for multiplexing electrical signals received from the array. The first sensing device 100-1 may be connected to a first channel 152-1 and the second sensing device 100-2 may be connected to a second channel 152-2. Referring to FIG. 20, the first WE 120-1, CE 140-1, and RE 130 may be connected to the first channel 152-1. The second WE 120-2, CE 140-2, and RE 130 may be connected to the second channel 152-2. The multiplexer 150 may be in two-way communication with the sensing circuitry 160. For example, the sensing circuitry can be configured to apply modulation signals to the array via the multiplexer. Output signals from the first and second channels may be transmitted to the sensing circuitry for simultaneous and multiplexed detection of the different target analytes present in the fluid sample.

The sensing circuitry 160 can be configured to take electrochemical measurements. In some embodiments, the sensing circuitry may comprise a potentiostat. The sensing circuitry may be capable of signal generation and signal conditioning. In some embodiments, the sensing circuitry may include converters such as analog-to-digital converters (ADC) and digital-to-analog converters (DAC). The sensing circuitry 160 can be configured to selectively apply a plurality of modulation signals to the two sensing devices 100-1 and 100-2 to enable detection of the plurality of different target analytes in the fluid sample. The sensing circuitry can be configured to individually and selectively control, activate, or modulate the two sensing devices. The plurality of modulation signals can be configured to aid in enhancing detection sensitivity of the different target analytes. The sensing arrays described herein can include any number of electrodes (e.g. working electrodes, counter electrodes, and reference electrodes) in various types of configurations. The sensing circuitry can be configured to individually and selectively control, activate, or modulate any number of sensing devices by applying different signals to the electrodes, for example as shown by the electrical field simulations in FIGS. 17A-17F.

As previously described, the first and second sensing devices 100-1 and 100-2 may comprise different capture reagents 124-1 and 124-2 that are configured to selectively bind to different target analytes 128-1 and 128-2 in a fluid sample. The selective binding is configured to effect changes to electron and ion mobility and charge accumulation in different regions of the semiconducting nanostructures 122-1 and 122-2 and the fluid sample. Each of the sensing devices can be configured to determine a presence and concentration of a different target analyte in the fluid sample based on detected changes to the electron and ion mobility and charge accumulation.

The sensing circuitry 160 can be configured for simultaneous acquisition and multiplexing of electrical signals from the sensing devices 100-1 and 100-2. The sensing circuitry is configured to analyze the electrical signals comprising of impedance and capacitance signals. The signals may be indicative of interfacial charge modulation comprising of the changes to the electron and ion mobility. The signals may include capacitance changes to space-charge regions formed in the semiconducting nanostructures upon binding of the different target analytes to the corresponding capture reagents. The changes may comprise simultaneous modulation to the ion mobility in one or more regions adjacent to the semiconducting nanostructures.

The sensing circuitry 160 can be configured to implement a plurality of electrochemical detection techniques for detecting the impedance changes and the capacitance changes. In some embodiments, the plurality of electrochemical detection techniques may comprise a modified EIS technique for measuring the impedance changes and Mott-Schottky technique for measuring the capacitance changes. The modified EIS technique is capable of distinguishing the electrical impedance signals from background noise at low concentrations of the different target analytes in the fluid sample.

The array 400 is capable of simultaneous and multiplexed detection of the different target analytes present in the fluid sample using the plurality of electrochemical detection techniques with aid of the sensing circuitry 160. The sensing circuitry 160 can be configured to perform the simultaneous and multiplexed detection by analyzing the electrical impedance and capacitance signals to determine the presence and concentration of each of the different target analytes. The sensing circuitry can be configured to perform the simultaneous and multiplexed detection substantially in real-time upon binding of the different target analytes to the corresponding capture reagents on the semiconducting nanostructures. The sensing circuitry can be configured to analyze the impedance and capacitance signals by concurrently analyzing a set of Nyquist plots obtained via the modified EIS technique and a set of Mott-Schottky plots obtained via the Mott-Schottky technique.

In some embodiments, the modified EIS technique may comprise (1) sectioning an interfacial charge layer for each of the two or more sensing devices into a plurality of spatial dielectric z-planes along a direction orthogonal to the interface between the fluid sample and the semiconducting nanostructures, and (2) probing each of the plurality of z-planes with a specific frequency selected from a range of frequencies. Specific binding of different target analytes to the corresponding capture reagents may occur at known spatial heights within the plurality of interfacial charge layers for the two or more sensing devices. The sensing circuitry can be configured to determine the presence and concentration of each of the different target analytes by measuring the capacitance and impedance changes at specific frequencies corresponding to their respective z-planes.

In some embodiments, the sensing circuitry 160 may be connected to a computing device 170. The sensing circuitry may or may not be part of the computing device. The computing device may be configured to process and/or display results obtained via the above-described electrochemical detection techniques. For example, the computing device can be configured to display an electrochemical signal response 180 which may include a set of Nyquist plots obtained via the modified EIS technique and/or a set of Mott-Schottky plots obtained via the Mott-Schottky technique. In some embodiments, the electrochemical signal response may be displayed on the computing device 170 for further analysis or data manipulation by a user.

In some embodiments, the first target analyte 128-1 may be cTnl antigen, and the first capture reagent 124-1 may be an antibody that is specific to the cTnl antigen. The second target analyte 128-2 may be cTnT antigen, and the second capture reagent 124-2 may be an antibody that is specific to the cTnT antigen. The semiconducting nanostructures 122-1 and 122-2 on the WEs 120-1 and 120-2 may comprise ZnO nanostructures. The linker reagent 126 may comprise a DSP linker. The surfaces of the ZnO nanostructures may be functionalized with the DSP linker for attaching the antibodies to the nanostructures. Accordingly, the first and second sensing devices can be used for electrochemical detection of the different cardiac biomarker Troponin isoforms cTnl and cTnT. Baseline electrical characterization of the array of sensing devices can be verified based on an electrochemical impedance response at a predefined frequency (e.g., 100 Hz). The detection of cTnl and cTnT in the sample can be achieved using the modified EIS and Mott-Schottky techniques described as follows.

In a conventional EIS technique, impedance changes occurring at the electrode-electrolyte solution interface can be identified and quantified. However, the challenge in using conventional EIS for protein detection has been the inability to distinguish the impedance signal from background noise as the concentration of the target protein diminishes in the complex test solutions such as human serum.

In the modified EIS technique described in various embodiments herein, a small AC voltage (for example <100 mV peak-to-peak) can be applied over a range of frequencies (e.g. from 1 Hz to 15 KHz) across the sensing electrodes (WEs) of a sensing device or an array of sensing devices. In the presence of a fluid on the sensing surface, an electrical double layer (EDL) is formed at the sensing electrode/fluid interface. The capacitive impedance of the EDL reflects the composition of the ions/biomolecules/interferents present at the interface. In conventional EIS, the total capacitive impedance of the EDL is measured and hence it is not possible to distinguish the signal from specific binding events and non-specific interactions, especially when the concentration of the target materials or analytes is very low as compared to the interferent material.

In the modified EIS technique disclosed herein, the EDL can be sectioned along the z-direction, i.e. in the orthogonal direction to the sensing electrode-electrolyte solution interface with subnanometer precision. Each spatial z-plane within the electrical double layer can be probed with a specific frequency. Since the specific binding of the protein with an immobilized antibody capture probe is expected to occur at a known spatial height within the EDL, protein binding even at ultra-low concentrations can be extracted with precision and accuracy by measuring the capacitive impedance changes at a specific frequency corresponding to the z plane in which the protein binding event occurs. The modified EIS technique disclosed herein is advantageous in that resolution is not diminished in the presence of complex media with high concentrations of interferent material.

In the modified EIS technique, the EDL at the sensing electrode/electrolyte buffer interface can be fragmented and analyzed at varying heights from the interface by measuring the impedance response at multiple frequency planes. Specific interactions between a target protein and its specific antibody capture probe can be selectively identified through a maximal change to the measured impedance at a specific frequency which maps to the height from the interface where antibody-target analyte binding happens. The use of the modified EIS technique can enhance specificity of detection. The use of ZnO can aid in achieving heightened sensitivity by leveraging the ionic and semiconducting nature of the semiconducting material. Also, the use of ZnO nanostructures can enhance signal response as a result of biomolecule confinement.

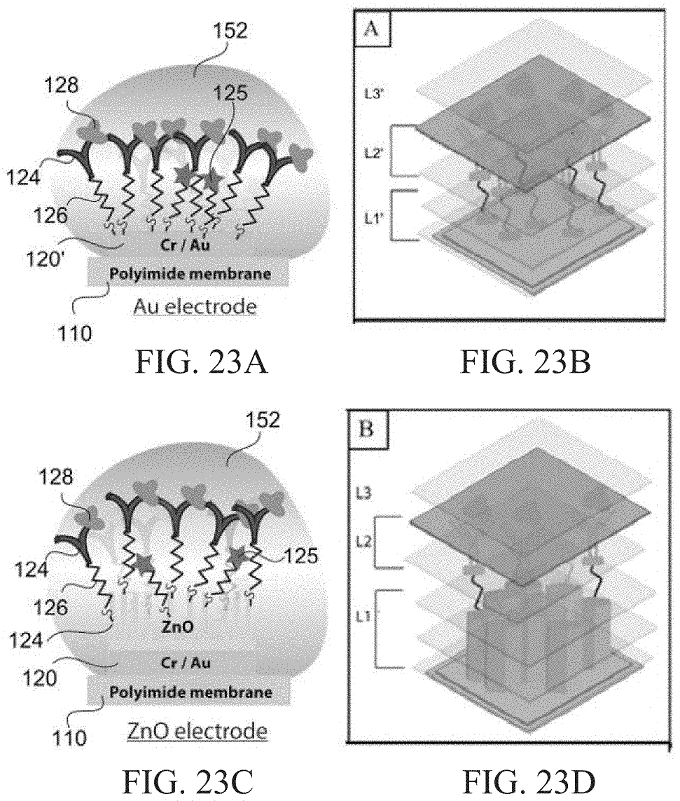

FIG. 23A illustrates fluid sample absorption onto a working electrode (WE) 120′ disposed on a substrate 110. The substrate may comprise a polyimide membrane. The WE 120′ may be a Au electrode having a Cr/Au surface finish. The WE 120′ may be substantially planar. The WE 120′ may be directly functionalized with a linker 126 that selectively immobilizes a capture reagent 124 (e.g., an antibody) that is specific for a target analyte 128 (e.g., an antigen). In some embodiments, a blocking reagent 125 may be optionally included to block excess binding sites on linker 126. A sample 152 comprising target analytes 128 may be introduced to the sensing device/array and adsorbed on the WE 120′. FIG. 23B illustrates z-plane fragmentation using a modified EIS technique on a plurality of Helmholtz planes at the planar sensor surfaces of FIG. 23A. Levels L1′, L2′ and L3′ as shown may correspond to different spatial z-planes which can be probed using logarithmic frequency scanning (e.g. ranging from 1 Hz-15 kHz).

FIG. 23C illustrates fluid sample absorption onto a working electrode (WE) 120 comprising semiconducting ZnO nanostructures 122 disposed on a substrate 110. The WE 120 may functionalized with the linker 126 that selectively immobilizes a capture reagent 124 (e.g., an antibody) that is specific for a target analyte 128 (e.g., an antigen). In some embodiments, a blocking reagent 125 may be optionally included to block excess binding sites on linker 126. A sample 152 comprising target analytes 128 may be introduced to the sensing device/array and adsorbed on the WE 120. FIG. 23D illustrates z-plane fragmentation using a modified EIS technique on a plurality of Helmholtz planes at the EDL interface at the nanostructured sensor surfaces of FIG. 23C. Levels L1, L2 and L3 as shown may correspond to different spatial z-planes which can be probed using logarithmic frequency scanning (e.g. ranging from 1 Hz-15 kHz).

Comparing FIGS. 23B and 23D, it can be observed that the height L1 of the semiconducting ZnO nanostructures is greater than the height L1′ of the planar Au electrode layer. Accordingly, the semiconducting ZnO nanostructures can increase the z-height or profile of the working electrode which is advantageous. For example, since the specific binding of a target analyte with an immobilized capture reagent is expected to occur at a known spatial height within the EDL, binding events at ultra-low concentrations can be extracted with precision and accuracy by measuring the capacitive impedance changes at a specific frequency corresponding to the z plane in which the protein binding event occurs. By probing the impedance over a larger L1′ plane, the modified EIS technique can maintain its resolution in the presence of complex media with a high concentration of interfering material.

The modified EIS technique can be used to fragment the EDL along the z direction with subnanometer precision by changing the frequency of measured response for stepwise changes to the applied potential within the electrochemical window of the ionic liquid (IL)/electrolyte. Recognition and detection of specific binding events for different protein biomarkers (e.g. cTn, NT-pro BNP, and CRP) in a multiplexed manner can be achieved as a result of dielectric permittivity modulation along the frequency spectrum due to the zwitterion stabilization effect of the ionic liquids in the EDL at the IL/ZnO electrode buffer interface. Bode analysis with collected impedance spectra can be used to identify the frequency range at which capacitive behavior is dominant. The identified frequency range in performing a Nyquist analysis can be used to quantify the effect of charge transfer for varying concentrations of a target biomolecule. Thus the ZnO surfaces can enhance biomolecule detection. The maximum impedance change from different assay steps can be used to design the calibration dose response curve to correlate the concentration of bound target biomolecules and the measured changes in impedance.

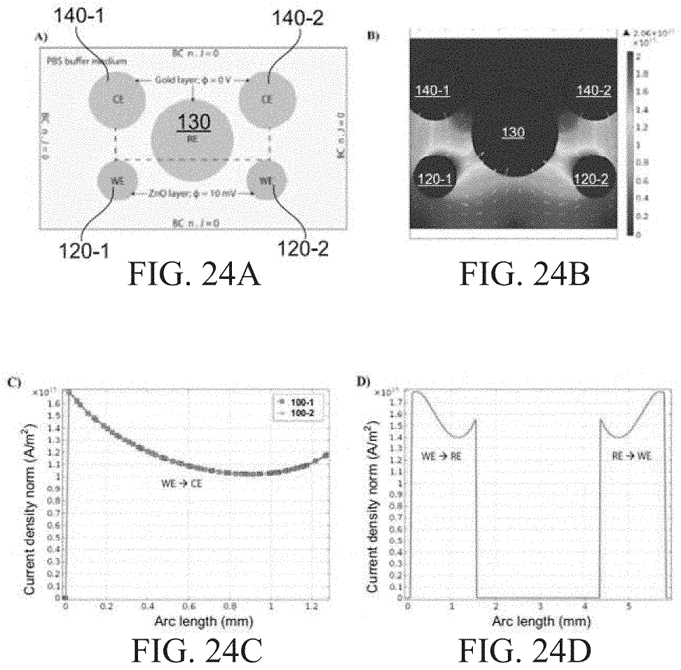

FIG. 24A shows a 2D schematic geometric model of the sensing array of FIG. 19 in COMSOL domain with applied boundary conditions. COSMOL Multiphysics is a finite element software that can be used to virtually simulate the real-time behavior of the sensing array to determine its performance. The simulation results can be used to optimize the design of the multiplexed sensing array to meet certain desired characteristics. The use of simulations can also help to reduce fabrication cost and time.

The COSMOL model encompasses the multi-electrode geometry constructed in three dimensional space. Simulations are performed using an AC/DC module with assumption of no magnetic field effects to establish that the first and second sensing devices of the array have the same baseline electrical performance. The geometric structures of each sensing device comprise three microelectrodes (WE, CE, and RE) built on polyimide substrate and surrounded by a rectangle made of PBS. Electrical properties of gold are assigned to both the counter electrodes (CEs) and the reference electrode (RE). The working electrodes (WEs) are assigned the semiconducting properties of ZnO. A constant applied potential of 10 mV is set at the WE. The boundary condition of both the RE and the CEs is set at zero potential. Electrical insulation with a von Neumann boundary condition (n.J=0) is applied to the PBS layer. The transient electric field is assumed to be confined within the multiplexed electrodes and the surrounding PBS medium and is governed by the following continuity equation.

$$∇ \cdot J = {Q}_{j} i . e . ∇ \cdot σ E = - \frac{\partial ρ}{\partial t}$$

where σ is the charge density. Based on Ohm's law, a relation between the current density, J (vector quantity) and the electric potential, V (scalar quantity) can be established. The electric field E, can be obtained from the following constitutive relation and the gradient of the scalar potential V as shown.

D=εₒεᵣE

E=−∇V

In the above equations, D is the displacement current, εₒ is the permittivity of free space and εᵣ is the relative permittivity of the material/electrolyte used. The discretization of the system into finite elements is based on physics-controlled mesh generation.

FIG. 24B shows the current distribution in the multiplexed sensing array for simulations performed with the above-described boundary conditions. The surface plot shows uniform distribution of current density between the electrodes of the sensing array. Maximum current density is observed near the surface of WEs which indicates that the output current response measured using a modified EIS technique is from the WEs. The direction of the white arrows corroborates that the electric field lines are directed away from the positive surface and that the performed simulations are correct.

FIG. 24C shows the variation in measured current density with distance between WE and CE in the sensing array along the vertical dotted lines depicted in FIG. 24A. FIG. 24D shows the variation in measured current density with distance between WE and RE in the sensing array along the horizontal dotted line depicted in FIG. 24A. The results indicate that both WEs exhibit the same performance along their surfaces and in each three electrode setup. For points that are measured farther away from the WE, current density decreases and with a highest value of 1.7×10¹⁵ A/m² observed at its surface. The simulation results indicate that both WEs exhibit the same baseline electrical performance under ideal conditions, and thus placement of the electrodes in the multiplexed sensing array has minimal to no variation. Surface modification of the WEs can perturb the charge distribution at the electrode/electrolyte interface. These perturbations are based on realignment of electrons or holes in the electrode surface and ions in the electrolyte solution. Thus, these charge perturbations can be leveraged towards designing the sensing devices/array described herein for multiplexed detection of multiple biomarkers.

Physicians currently use a combination of imaging and laboratory analysis for disease diagnosis in a clinical setting. Samples from patients can be tested for a multitude of biomolecular markers. This type of analysis, while precise and repeatable, requires significant processing time and hence not applicable for POC diagnostics. The development of successful sensing device for POC disease diagnostics relies on four major attributes: rapid detection, sensitivity of detection, specificity of detection, and ease of use. The incorporation of these key features can allow clinicians to efficiently provide the necessary feedback and care to their patients regarding diagnosis, prognosis and response to therapy. However, current handheld POC devices for cardiac biomarkers often lack the ability to provide diagnostics in real-time and with high accuracy and consistency at patient bedside outside the ED and hospital environment such as primary care, assisted/independent living care, and ambulatory environments.

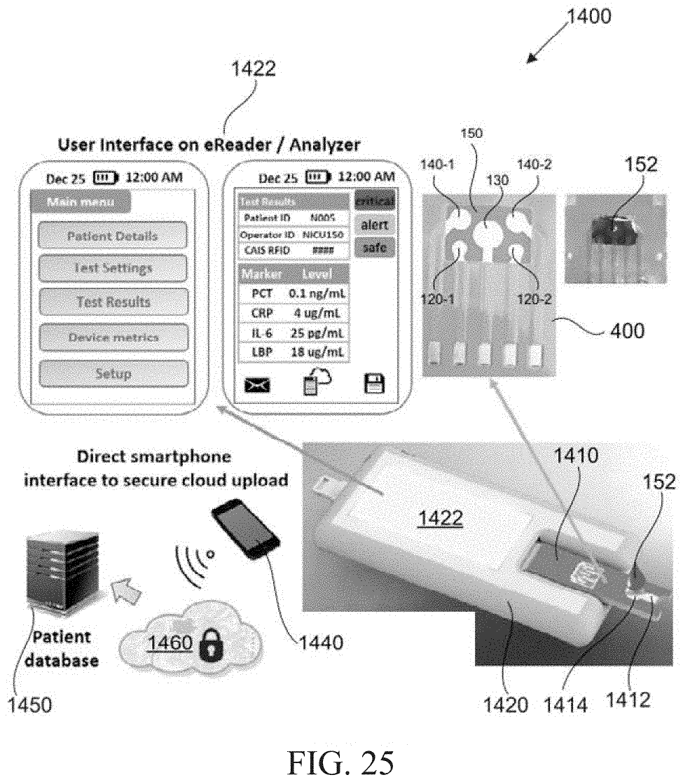

The above needs can be addressed using the sensing platform shown in FIG. 25 in accordance with some embodiments. The sensing platform may be configured to perform immunoassays as described elsewhere herein.

Referring to FIG. 25, a sensing platform 1400 may include a test strip 1410 and a diagnostic reader device 1420. The test strip may include a sensing device or sensing array. For example, the sensing array 400 shown in FIG. 19 may be provided on the test strip. In some cases, the test strip is composed of a material comprising a plurality of capillary beds such that, when contacted with a sample fluid, the sample fluid is transported laterally across the test strip. The sample fluid may be flowed along a flow path of the test strip from a proximal end to the distal end of the test strip. The sample is flowed by capillarity or wicking. Non-limiting examples of test strips may include porous paper, or a membrane polymer such as nitrocellulose, polyvinylidene fluoride, nylon, Fusion 5™, or polyethersulfone.

The test strip 1410 may also include a wicking pad 1412. The wicking pad may be composed of, e.g., filter paper. Other optional features may include a cover for supporting and/or protecting the test strip. The cover may be composed of a sturdy material such as plastic (e.g., high-impact polystyrene). The cover may, e.g., may protect from inadvertent splashing of a sample onto the test strip (e.g., when the device is applied to a urine stream), and to protect the sensitive areas of the test strip (e.g., the sensing array). The cover may include various openings or windows along the test strip. For example, the cover may include a sample application zone 1414 for applying the fluid sample 152 to the wicking pad 1412.

The test strip may comprise a zone and/or region for conducting an immunoassay. The test strip may define a flow path. The zone and/or region for conducting immunoassays in accordance with the disclosure may be positioned along a flow path of the test strip such that a fluid sample may be flowed (e.g., by capillarity) from the sample application zone 1414 on a proximal end of the strip to a test zone 150 of the sensing array 400. In some alternative embodiments, instead of transporting the sample via capillary flow, the fluid sample 150 may be dispensed (e.g. by pipetting) directly onto the test zone 150.

A test strip may comprise sensing array that are functionalized to detect analytes of interest. Test strips comprising different types of sensing arrays can be provided. The sensing arrays may have different sensing electrode materials (e.g. semiconducting materials), linker chemistries, and capture reagents for binding with a variety of different target analytes, depending on the desired sensing/biosensing application and end physiological state to be predicted.

The diagnostic reader device 1420 can be configured for use with the test strip. The reader device can be a hand-held electronic device. The reader device can be configured to receive the test strip. For example, the test strip can be inserted into a receiving port or chamber of the reader device, thereby establishing electrical connection with the reader device. The reader device may comprise, for example the multiplexer 150, sensing circuitry 160, and/or computing device 170 shown in FIG. 20. The reader device can be configured to perform electro-analytical diagnostics on the test strip substantially in real-time. The electro-analytical diagnostics may include collecting and analyzing the electrochemical signal responses as described elsewhere herein.

In the example of FIG. 25, the test strip is shown inserted into the receiving chamber of the reader device. The reader device can generate measurement results (e.g., concentration or relative amounts of analytes present in the sample) from a completed assay performed on the test strip, as described throughout. The reader device can display the measurement results on a screen 1422 of the reader device. In some embodiments, data containing the measurement results can be transmitted from the reader device to a mobile device 1440 and/or to a server. The data may be transmitted via one or more wireless or wired communication channels. The wireless communication channels may comprise Bluetooth®, WiFi, 3G, and/or 4G networks.

In some embodiments, the data containing the measurement results may be stored in a memory on the reader device when the reader device is not in operable communication with the mobile device and/or the server. The data may be transmitted from the reader device to the mobile device and/or the server when operable communication between the reader device and the mobile device and/or the server is re-established.

A network 1460 can be configured to provide communication between the various components of the embodiments described herein. The network may be implemented, in some embodiments, as one or more networks that connect devices and/or components in the network layout for allowing communication between them. For example, one or more diagnostic test devices, mobile devices and/or servers may be in operable communication with one another over a network. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth®, Near Field Communication (NFC) technologies, networks based on mobile data protocols such as General Packet Radio Services (GPRS), GSM, Enhanced Data GSM Environment (EDGE), 3G, 4G, or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

One or more reader devices, mobile devices and/or servers may be connected or interconnected to one or more databases 1450. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments. In some embodiments, the databases 1450 may include patient databases.

In some embodiments, one or more graphical user interfaces (GUIs) 1422 may be provided on the reader device 1420. Additionally or optionally, the GUIs may be provided on the mobile device 1440. The GUIs may be rendered on a display screen. A GUI is a type of interface that allows users to interact with electronic devices through graphical icons and visual indicators such as secondary notation, as opposed to text-based interfaces, typed command labels or text navigation. The actions in a GUI are usually performed through direct manipulation of the graphical elements. In addition to computers, GUIs can be found in hand-held devices such as MP3 players, portable media players, gaming devices and smaller household, office and industry equipment. The GUIs may be provided in a software, a software application, a web browser, etc. The GUIs may be provided through a mobile application. The GUIs may be rendered through an application (e.g., via an application programming interface (API) executed on the mobile device). The GUIs may show images that permit a user to monitor levels of analytes of interest.

As depicted in FIG. 25, the sensing platform may further comprise means for transmitting data generated by the reader device and sensing array. In some cases, the data may be transmitted to and/or read from a mobile device (e.g., a cell phone, a tablet), a computer, a cloud application or any combination thereof. The data may be transmitted by any means for transmitting data, including, but not limited to, downloading the data from the system (e.g., USB, RS-232 serial, or other industry standard communications protocol) and wireless transmission (e.g., Bluetooth®, ANT+, NFC, or other similar industry standard). The information may be displayed as a report 1430. The report may be displayed on the screen 1422 of the reader device 1420 or a computer. The report may be transmitted to a healthcare provider or a caregiver. In some instances, the data may be downloaded to an electronic health record. Optionally, the data may comprise or be part of an electronic health record. For example, the data may be uploaded to an electronic health record of a user of the devices and methods described herein. In some cases, the data may be transmitted to a mobile device and displayed for a user on a mobile application.

Data collected by and transmitted by the reader device may include results of the immunoassay test performed on the test strip. For example, the data may include the concentrations of different analytes present in a sample. The concentrations may include relative concentrations or absolute concentrations. For example, the GUI 1422 in FIG. 25 shows the levels of different markers such as PCT, CRP, IL-6, and LBP. The data may also include an outcome such as a diagnostic outcome or a prognostic outcome. The data may also include alerts to the user (e.g. critical, alert, safe). In some cases, the alerts may be color-coded to generate awareness to the user.

Additional data that may be transmitted by the reader device include, without limitation, patient information/details, test settings, device metrics, device setup, time and date of the immunoassay tests, system status (testing temperature, battery status, system self-testing and calibration results), error codes or error messages, etc.

Current handheld POC devices typically offer detection of a single biomarker on a single parameter test strip or cartridge. In contrast, the sensing platform 1400, particularly the sensing array 400 with multiplexer 150 and sensing circuitry 160, can provide simultaneous detection of multiple biomarkers for rapid diagnostic and prognostic on a single electrochemical test strip. The simultaneous and multiplexed detection of multiple biomarkers on a single electrochemical test strip obviates the need to use multiple discrete test strips for detecting different biomarkers.

Additionally, the sensing platform 1400 is capable of analyzing multiple biomarkers using very small volumes (e.g. 30 μL) of the fluid sample (e.g. finger-pricked blood) performed substantially in real-time at the patient's bedside.

The sensing platform can lower health care costs through reduced cost of the disposable test strip for multiple biomarker detection, and providing diagnostic and prognostic analysis at the patient bedside in non-clinical environments thus generating savings on physician costs and hospitalization costs. The data analyzed can be securely transmitted to a secure cloud server for the primary physician managing the patient to be able to access, review, and manage guidance and therapies. In the example of FIG. 25, the sensing platform can aid in assessing congestive heart failure (CHF) risk based on the measured levels of the different markers, and is therefore of immediate benefit to primary care and ED physicians. Furthermore, rapid availability of the immunoassay testing can facilitate a rule-out protocol in a busy emergency department.

An example of a POC application using the sensing platform 1400 is next described. A disposable sensing array comprising of IL/ZnO hybrid liquid/solid semiconducting electrode, is functionalized with antibodies that are receptors for the panel of protein biomarkers to be tested. A test sample comprising of ≤20 μL (1-2 drops) blood serum, blood plasma can be dispensed onto the sensor electrodes through standard capillary wicking methods common to lateral flow immunoassays, which yields immunoassay formation at the RTIL/ZnO-buffer interface. The sensing array can be connected to sensing circuitry in the reader device. The sensing circuitry may include a potentiostat, and the reader device may be a hand-held electronic device. After an incubation period sufficient for diffusion limited processes, the sensing circuitry in the reader device measures the impedance over a range of frequencies in the electrochemical window of the RTIL. Based on reference sigmodial calibration, the concentration of a panel of protein biomolecules (e.g., cTn, NT-proBNP, and CRP) can be determined and displayed on the reader device. The sensing platform 1400 is capable of ultrasensitive detection of Troponin and NT-proBNP cardiac markers with high specificity and minimal cross-reactivity in human serum samples. The protein binding and detection process for Troponin and NT-proBNP can be achieved by using a single capture immunoassay (e.g., primary monoclonal antibody-antigen interaction) without the use of any secondary antibody.

In another embodiment, the sensing platform 1400 can be used in aptasensing for K+ detection. Aptamer oligonucleotides that contain single or multiple guanine-rich segments are known to form specific four-stranded helical conformations in solution with an extraordinary selectivity for potassium. In the absence of potassium, the aptamer containing multiple guanine-rich segments adopts a random-coil structure that upon exposure to potassium ion (K+) solution displaces the equilibrium in favor of the G-quadruplex form, the G-quadruplex being a conformation of guanine-rich DNA resulting from the association of sets of four guanine residues into planar arrays. The sensing platform 1400 is capable of higher sensitivity and specificity in the detection of aptamers, as compared to the use of standard ion-selective electrodes for electrolyte sensing.

Accordingly, the sensing platform 1400 can be used for affinity-based impedimetric sensing of troponin (cTnl, cTnT) and NT-proBNP using specific antibodies and affinity based amperometric sensing of K+ and other similar ions using specific aptamers from human blood. As previously described, the human blood can be transported by capillary action on the test strip to the test zone. The test strip can be inserted into the reader device to provide rapid diagnostic and therapeutic response to a physician at the patient's bedside. The sensing platform 1400 can be used for near-patient cardiovascular diagnosis and assessment in primary care, EDs, assisted/independent living care, and ambulatory environments, towards real-time detection and monitoring levels of a panel of cardiac biomarkers (cTnl, NT-proBNP) and sodium, potassium, calcium levels from finger-pricked capillary blood.

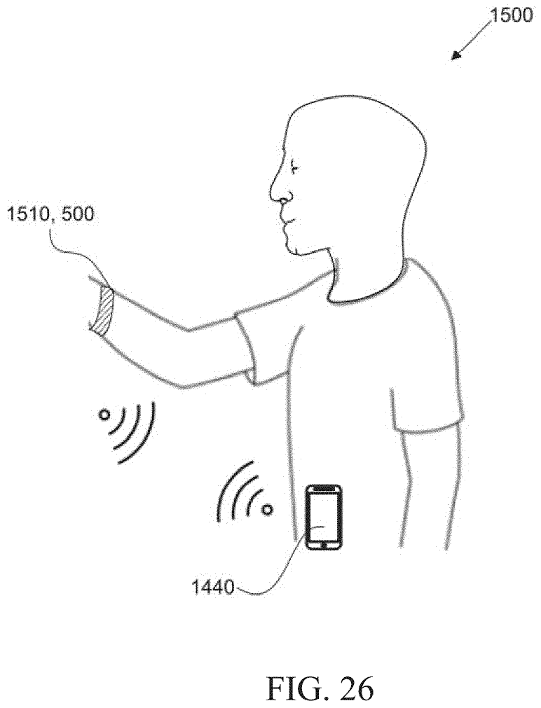

In some embodiments, the sensing devices and arrays described herein may be provided on a wearable sensing platform 1500 as shown in FIG. 26. For example, the sensing system 500 shown in FIG. 20 may be provided on a wearable device 1510. Examples of wearable devices may include smartwatches, wristbands, glasses, gloves, headgear (such as hats, helmets, virtual reality headsets, augmented reality headsets, head-mounted devices (HMD), headbands), pendants, armbands, leg bands, shoes, vests, motion sensing devices, etc. The wearable device may be configured to be worn on a part of a user's body (e.g., a smartwatch or wristband may be worn on the user's wrist). The wearable device may include one or more types of sensors. Examples of types of sensors may include heart rate monitors, external temperature sensors, skin temperature sensors, capacitive touch sensors, sensors configured to detect a galvanic skin response (GSR), and the like.

In some embodiments, the sensing system on the wearable device can be capable of transdermally monitoring alcohol content. For example, the sensing system can be configured to monitor blood alcohol levels in real time from ambient perspired sweat. A wearable device (e.g. in the form of a bracelet) can unobtrusively house the sensing systems described herein for simultaneous monitoring of Ethanol and paired Ethyl glucuronide (EtG), Ethyl Sulfate (EtS), Phosphatidylethanol (PEth) levels from ambient perspired sweat. The wearable device can be capable of transdermal measurement of blood alcohol content by detecting and quantifying ethanol paired with simultaneous detection of non-volatile metabolites EtG, EtS, PEth, etc. from ambient perspired sweat. This multi-parameter information can be transmitted via wireless data transmission from the wearable device to portable, hand-held devices such as a smart phone. EtG and EtS are stable, non-oxidative metabolites of alcohol and can be detected in body fluids including sweat. Simultaneous detection of Ethanol and paired EtG, EtS in perspired sweat using unobtrusive and comfortable wearable devices can offer the potential to dramatically improve the ability to accurately assess the responses to treatments, and build longer term behavioral patterns of the individual which is of significant value for research and clinical purposes.

The wearable sensing platform can provide enhanced ability for users and health professionals to collect consumption and exposure assessment data in a variety of scenarios, leading to a greater understanding of the relationship between personal alcohol consumption and exposures and to user physiology, psychology, and disease origins. This can be advantageous in providing assessments for susceptible and at-risk groups, such as young adults, recovering addicts, and people with existing chronic diseases. The wearable sensing platform can be configured to differentiate results for varying alcohol consumption in varying social settings, while collecting data from individuals at the point of exposure. In some cases, wearable sensing platform can also account for individual mobility/variability as people move though different, possibly spatially heterogeneous environments (e.g. via GPS triangulation).

Enzyme-based ethanol sensing technologies are generally based on monitoring of NADH in the case of ADH based sensing devices and O₂ consumption or H₂O₂ production in the case of alcohol oxidase (AOX) sensing devices. Alcohol dehydrogenase (ADH; Alcohol:NAD⁺ oxidoreductase, EC 1.1.1.1) catalyzes the reversible oxidation of primary aliphatic and aromatic alcohols other than methanol. Alcohol oxidase (AOX; Alcohol:O₂ oxidoreductase, EC 1.1.3.13) catalyzes the conversion of alcohols into corresponding aldehydes or ketones, but not the reverse reaction similar to that catalyzed by the ADH (Scheme 1a). AOX requires flavin-based cofactors, while ADH requires NAD-based cofactors. The FAD in AOX is avidly associated with the redox center of the enzyme and is involved in transferring the hydride ion originated from alcohol substrate to molecular oxygen leading to the formation of H₂O₂. The oxidation of alcohols by AOX is irreversible, due to the strong oxidizing character of O₂. The NAD⁺ (or NADP⁺) involved in ADH catalysis is a strong oxidizing agent that accepts the hydride ion directly from the substrate during the catalysis and generating the corresponding reduced form, NADH/NADPH.

In some embodiments, the sensing system on the wearable device 1510 is configured for catalytic sensing using amperometric methods, which can be used to detect the presence of alcohol in perspired human sweat through either of the above described mechanisms. The ADH or AOX enzyme would be bound to the sensing electrode surface through the linker chemistry, and NAD⁺ or FAD⁺ co factor would be applied to the sensing electrode surface. The electrochemical reaction being endothermic (negative AG) will primarily proceed in the presence of the catalyst and under an applied potential. Thus when alcohol is present in the solution, the reaction with NAD⁺ or FAD⁺ takes place at the sensing electrode surface where the catalyst ADH or AOX is respectively bound and the resulting electrons transfer is measured and used to quantify in real-time the amount of alcohol present in the solution.

In some embodiments, the sensing system on the wearable device 1510 is configured for EtG detection in pooled human sweat using affinity based sensing of bound specific antibodies to Au and ZnO surfaces using the linker chemistry and with the modified EIS technique described elsewhere herein.

The sensing system can employ affinity based impedimetric sensing of EtG and EtS, and PEth using specific antibodies and catalytic enzymatic based amperometric sensing of alcohol with affinity bound enzymes on a multi-configurable electrochemical sensing platform with human sweat sample. This can be used to monitor personal alcohol consumption and abstinence, and can also be used to establish behavioral patterns in social settings.

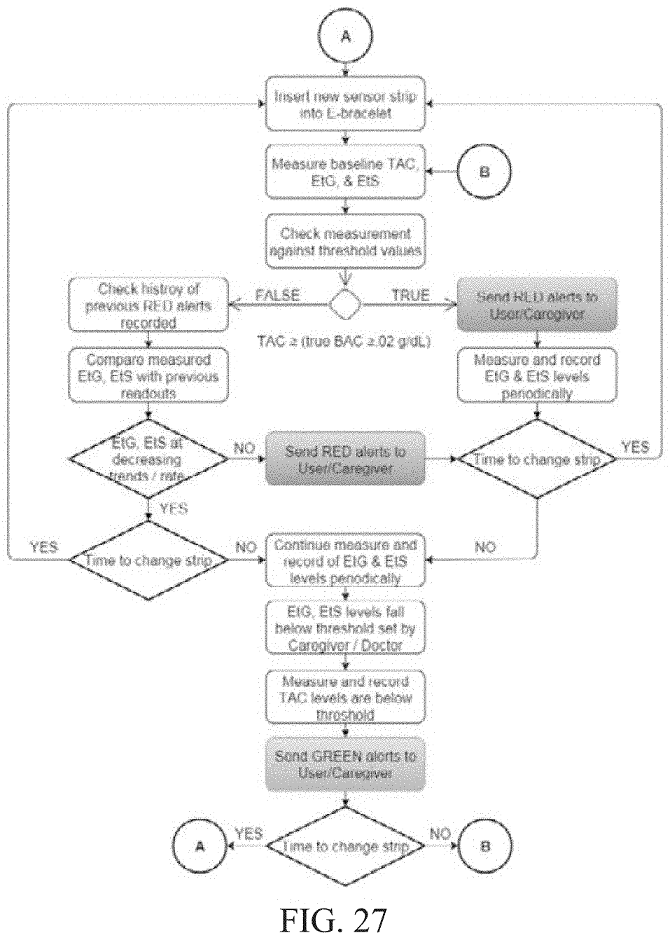

FIG. 27 is a flowchart showing a method for continuous, real-time detection of alcohol, EtG, and EtS in accordance with some embodiments. A wearable device (e.g. an e-bracelet) can be configured to receive and perform an immunoassay on a test strip. A test strip containing bodily fluids may be inserted into the wearable device, and the total alcohol content (TAC), EtG, and EtS are measured. Next, the measurements are compared against threshold values. If the TAC is greater than or equal to the threshold values, a negative alert may be sent to the user and/or to a caregiver, while the wearable device continues to measure and record the EtG and EtS levels periodically. Conversely, if the TAC is less than the threshold values, the history of previously recorded negative alerts may be analyzed. The current measured EtG and EtS levels may be compared with previous readouts, to determine if there is an increasing or decreasing trend/rate. If there is an increasing trend/rate in the measured EtG and EtS levels, a negative alert may be sent to the user/caregiver. If there is a decreasing trend/rate in the measured EtG and EtS levels, the wearable device may continue to measure and record the EtG and EtS levels periodically. When the measured EtG and EtS levels falls below predefined values set by the user/caregiver, the TAC may be measured to confirm that TAC levels are below the threshold values, and a positive alert may be subsequently sent to the user/caregiver. In some embodiments, the method may include various steps at which the user is notified by the wearable device whether the test strip needs to be changed. A person of ordinary skill in the art will recognize many variations, alterations and adaptations based on the disclosure provided herein. For example, additional steps may be added as appropriate. Some of the steps may comprise sub-steps. Some of the steps may be automated (e.g., autonomous sensing), whereas some of the steps may be manual (e.g., requiring manual handling, input or responses from a user). The systems and methods described herein may comprise one or more instructions to perform at least one or more steps of method 1500.

Various modifications can be made to the sensing devices or arrays described elsewhere herein. In some cases, the sensing devices or arrays can be modular in nature and customized for different sensing applications. For example, a substrate can be modified to receive and interchange thereon a plurality of discrete sensors. The plurality of discrete sensors may comprise different capture reagents that are configured to selectively bind to different target analytes in a fluid sample. Providing a practically unlimited diversity of discrete sensors can result in better health monitoring and outcomes for users, for a variety of biological and chemical sensing applications.

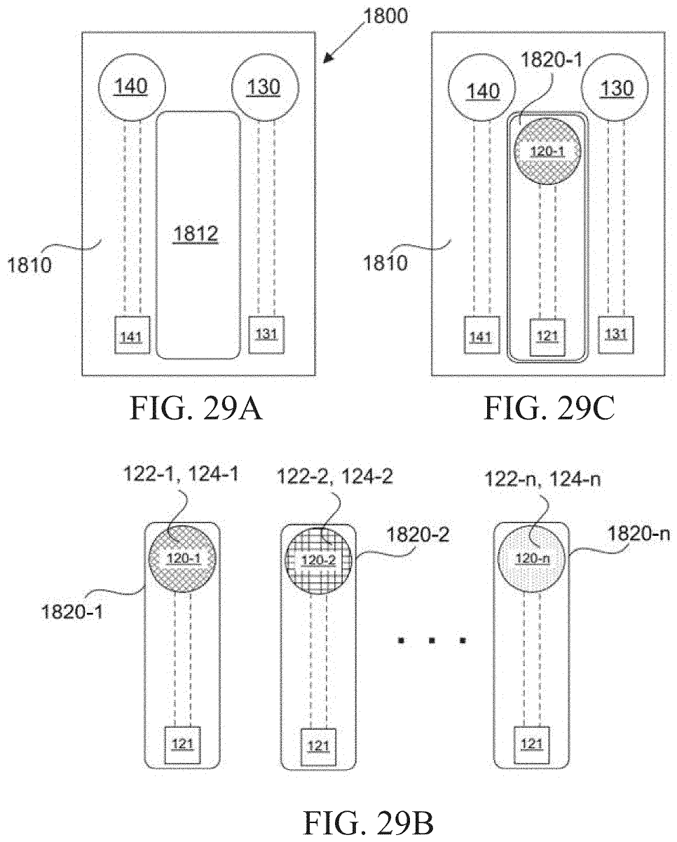

FIGS. 29A-C show an example of a modular sensing device 1800 in accordance with some embodiments. The device 1800 can be configured to detect one or more targets in a fluid sample. The device may include a base module 1810. The base module 1810 may be similar to the substrate (e.g. 110) described elsewhere herein except the base module comprises a receiving portion 1812. The receiving portion may include a recess, cavity, or slot. The base module can be configured to releasably couple to one or more discrete sensors 1820 via the receiving portion 1812.

The discrete sensor(s) are configured to be mechanically and electrically coupled to the base module. The discrete sensor(s) can be used to determine a presence and concentration of one or more target analytes in a fluid sample based on detected changes to electron and ion mobility and charge accumulation when the discrete sensor(s) are coupled to the base module and the fluid sample is applied to the sensing device.

The base module 1810 may include a plurality of electrodes. For example, the base module may include at least one reference electrode (e.g. 140) and at least one ground electrode (e.g. 130). In some embodiments, the receiving portion 1812 may be located in a region between a ground electrode 130 and a reference electrode 140.

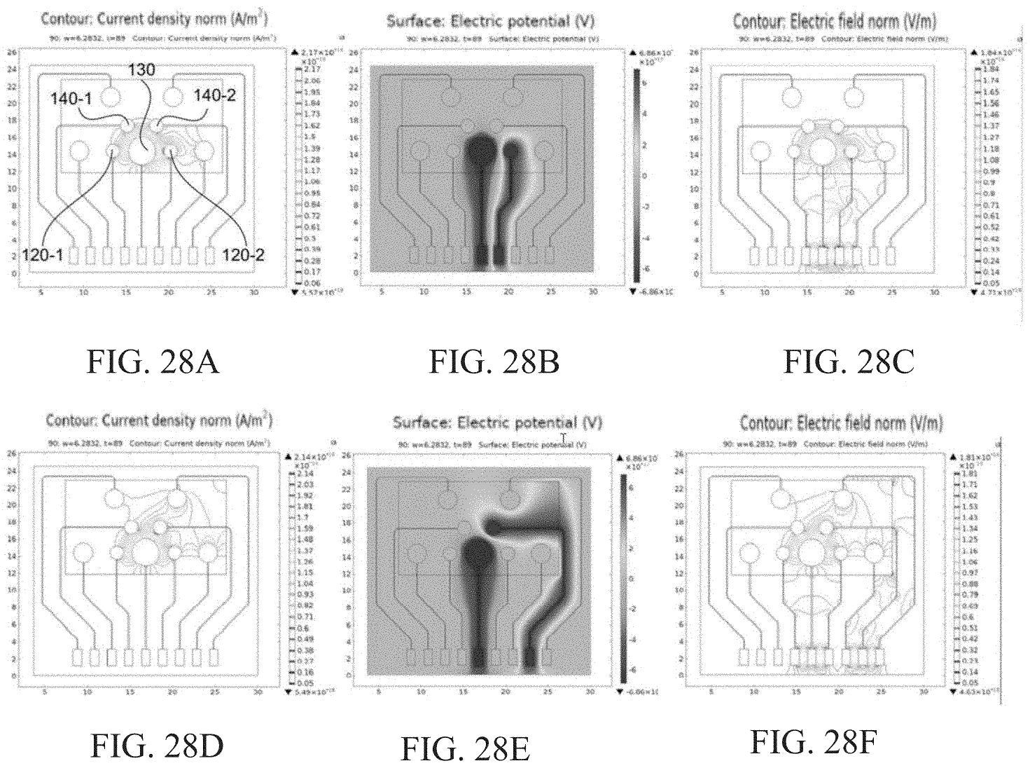

FIG. 28B shows a plurality of discrete sensors 1820-1 through 1820-n that can be interchangeably coupled to the base module of FIG. 28A. The plurality of discrete sensors can be configured to be interchanged and/or mounted onto the base module using a quick release mechanism and/or without the use of tools. FIG. 28C shows an example of a first discrete sensor 1820-1 being coupled to the base module 1810 via the receiving portion 1812.

Referring to FIG. 28B, each of the discrete sensors 1820 may comprise a working electrode 120 having a plurality of semiconducting nanostructures 122 disposed thereon, and a capture reagent 124 attached to the semiconducting nanostructures. The discrete sensors may include the same or different types of semiconducting nanostructures. The discrete sensors may comprise different capture reagents (124-1 through 124-n) that are configured to selectively bind to different target analytes in a fluid sample. The selective binding is configured to effect changes to the electron and ion mobility and charge accumulation in different regions of the semiconducting nanostructures and the fluid sample. The plurality of discrete sensors can be used for determining the presence and concentration of the different target analytes in the fluid sample, as described in many embodiments elsewhere herein.

In some embodiments, a first discrete sensor may be releasably coupled to the base module thereby electrically and mechanically connecting the first discrete sensor to the base module. Next, a fluid sample suspected to contain a first target analyte may be applied to the modular sensing device. The first discrete sensor can be used to determine a presence and concentration of the first target analyte in the fluid sample based on detected changes to electron and ion mobility and charge accumulation specific to the first target analyte. The first discrete sensor may be detached from the base module after the presence and concentration of the first target analyte has been determined.

Next, a second discrete sensor may be releasably coupled to the base module thereby electrically and mechanically connecting the second discrete sensor to the base module. Another fluid sample suspected to contain a second target analyte may be applied to the modular sensing device. The second discrete sensor can be used to determine a presence and concentration of the second target analyte in the fluid sample based on detected changes to the electron and ion mobility and charge accumulation specific to the second target analyte.

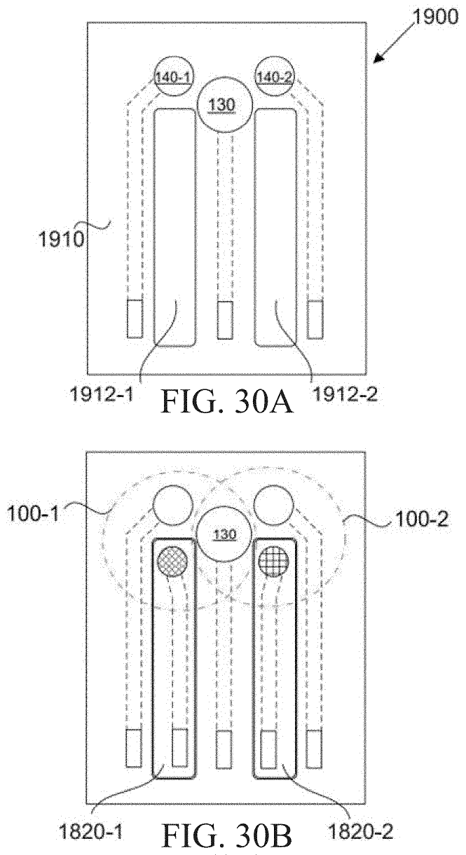

The modular sensing device of FIGS. 29A-C may be modified into a modular sensing array for example as shown in FIGS. 30A and 30B. A modular sensing array 1900 can be configured for simultaneous and multiplexed detection of two or more target analytes in a fluid sample. The array may include a base module 1910 configured to releasably couple to two or more discrete sensors. In the example of FIGS. 30A-B, the base module may comprise (1) a first receiving portion 1912-1 configured to couple to a first discrete sensor 1820-1, and (2) a second receiving portion 1912-2 configured to couple to a second discrete sensor 1820-2. The discrete sensors 1810-1 and 1810-2 are configured to be mechanically and electrically coupled to the base module. Each of the discrete sensors may comprise a working electrode 120 having a plurality of semiconducting nanostructures 122 disposed thereon, and a capture reagent 124 attached to the semiconducting nanostructures. The plurality of discrete sensors comprises different capture reagents that are configured to selectively bind to different target analytes in a fluid sample. The selective binding is configured to effect changes to the electron and ion mobility and charge accumulation in different regions of the semiconducting nanostructures and the fluid sample. The discrete sensors can be used to determine a presence and concentration of at least two different target analytes in the fluid sample based on detected changes to electron and ion mobility and charge accumulation when the discrete sensors are coupled to the base module and the fluid sample is applied to the sensing array.

The base module may comprise at least one reference electrode and at least one counter electrode. For example, the base module may comprise counter electrodes 140-1 and 140-2, and a common reference electrode 130. A first sensing device 1800-1 can be formed by coupling the first discrete sensor 1820-1 to the first receiving portion 1812-1. The first sensing device 1800-1 may comprise the first counter electrode 140-1, the working electrode 120-1, and the reference electrode 130. A second sensing device 1800-2 can be formed by coupling the second discrete sensor 1820-2 to the second receiving portion 1812-2. The second sensing device 1800-2 may comprise the second counter electrode 140-2, the working electrode 120-2, and the reference electrode 130. Accordingly, the first and second sensing devices 1800-1 and 1800-2 may share a common reference electrode. The first sensing device 1800-1 can be configured to determine the presence and concentration of a first target analyte, and the second sensing device 1800-2 can be configured to determine the presence and concentration of a second target analyte, similar to the embodiments described elsewhere herein.

In some embodiments, a method of using a modular sensing array for detecting one or more target analytes in a fluid sample may include providing a base module configured to releasably couple to one or more discrete sensors. The method may also include coupling the one or more discrete sensors to the base module thereby electrically and mechanically connecting said discrete sensors to the base module. The method may further include applying the fluid sample to the modular sensing array, and using the one or more discrete sensors to determine a presence and concentration of the one or more target analytes in the fluid sample based on detected changes to electron and ion mobility and charge accumulation specific to each of the one or more target analytes.

In some embodiments, the above method may include coupling a first discrete sensor and a second discrete sensor to the base module thereby electrically and mechanically connecting the first and second discrete sensors to the base module. A fluid sample suspected to contain a first target analyte and a second target analyte may be applied to the modular sensing array. The first discrete sensor can be to determine a presence and concentration of the first target analyte in the fluid sample based on detected changes to electron and ion mobility and charge accumulation specific to the first target analyte. Similarly, the second discrete sensor can be used to determine a presence and concentration of the second target analyte in the fluid sample based on detected changes to the electron and ion mobility and charge accumulation specific to the second target analyte.

Further provided herein are kits which may include any number of immunoassay test devices and/or reader devices of the disclosure. In one aspect, a kit is provided for determining qualitatively or quantitatively the presence and concentration of at least a first analyte and a second analyte in a fluid sample, the kit comprising: a) a sensing device or array according to one or more embodiments of the disclosure; and b) instructions for using the kit.

In some cases, a kit may provide a sensing device or array to enable a user to conduct a test on more than one occasion. In some cases, a kit may include a plurality of test strips each configured for a single use (i.e., are disposable). A kit may include a plurality of test devices to enable a user to perform a test once a day, once every 2 days, once every 3 days, once every 4 days, once every 5 days, once every 6 days, once every week, once every 2 weeks, once every 3 weeks, once every 4 weeks, once every 5 weeks, once every 6 weeks once every 7 weeks, once every 8 weeks or more.

In some cases, kits may include a plurality of immunoassay test devices, each capable of detecting different analytes. In some embodiments, kits may include a plurality of discrete sensors for detecting different analytes. In a particular embodiment, a kit may include the sensing array disclosed herein, that is capable of detecting the presence of cTnl and/or cTnT, NT-proBNP, and CRP in a biological sample such as blood. In another particular embodiment, a kit may include a sensing array disclosed herein, that is capable of detecting the presence and concentration of alcohol content, EtG, and EtS in a biological sample such as sweat.

In some cases, kits can be provided with instructions. The instructions can be provided in the kit or they can be accessed electronically (e.g., on the World Wide Web). The instructions can provide information on how to use the devices and/or systems of the present disclosure. The instructions can provide information on how to perform the methods of the disclosure. In some cases, the kit can be purchased by a physician or health care provider for administration at a clinic or hospital. In other cases, the kit can be purchased by the subject and self-administered (e.g., at home). In some cases, the kit can be purchased by a laboratory.

Kits may further comprise a diagnostic reader device or wearable device of the disclosure. The diagnostic reader device or wearable device may be configured to be used with the sensing devices or arrays of the disclosure. The diagnostic reader device or wearable device may be configured to be in operable communication with the sensing devices or arrays.

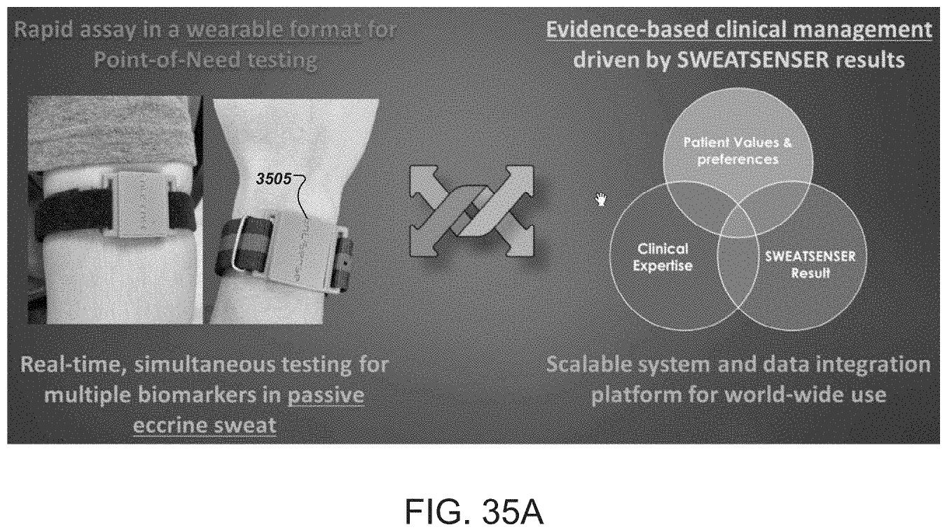

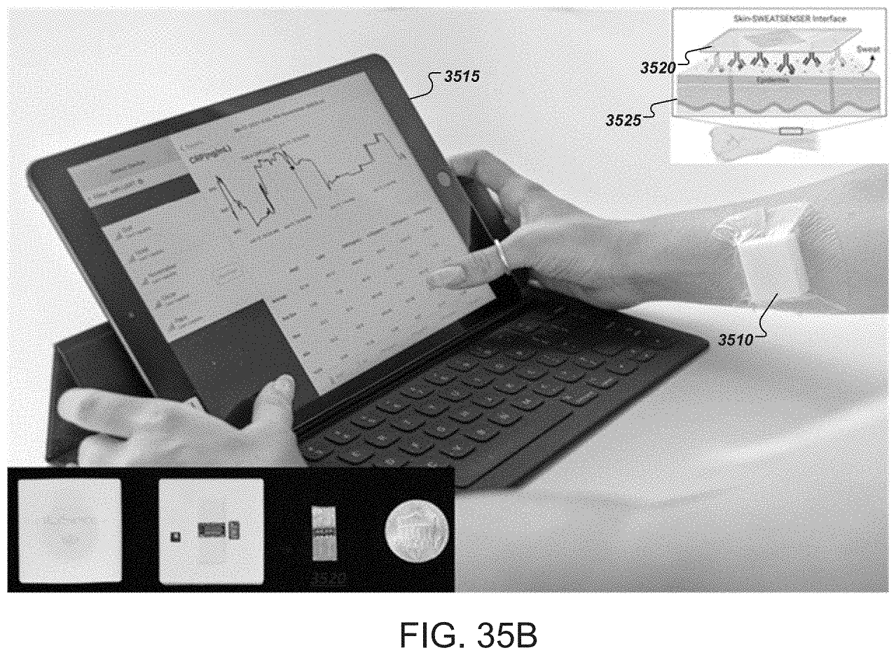

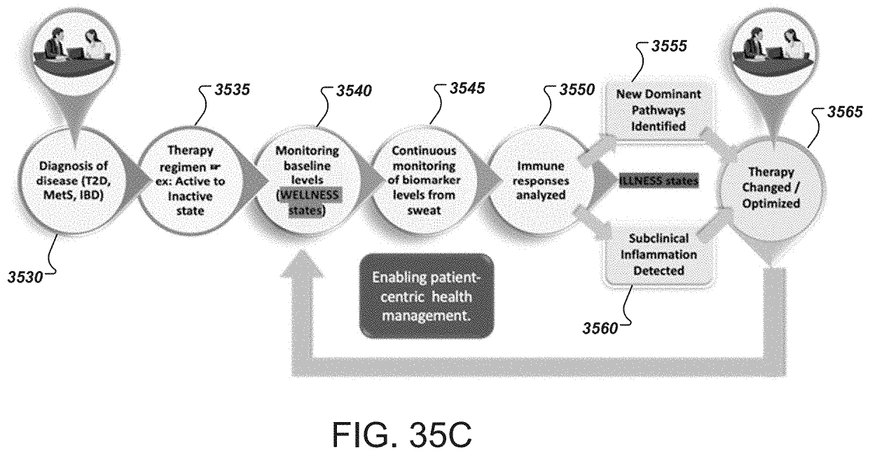

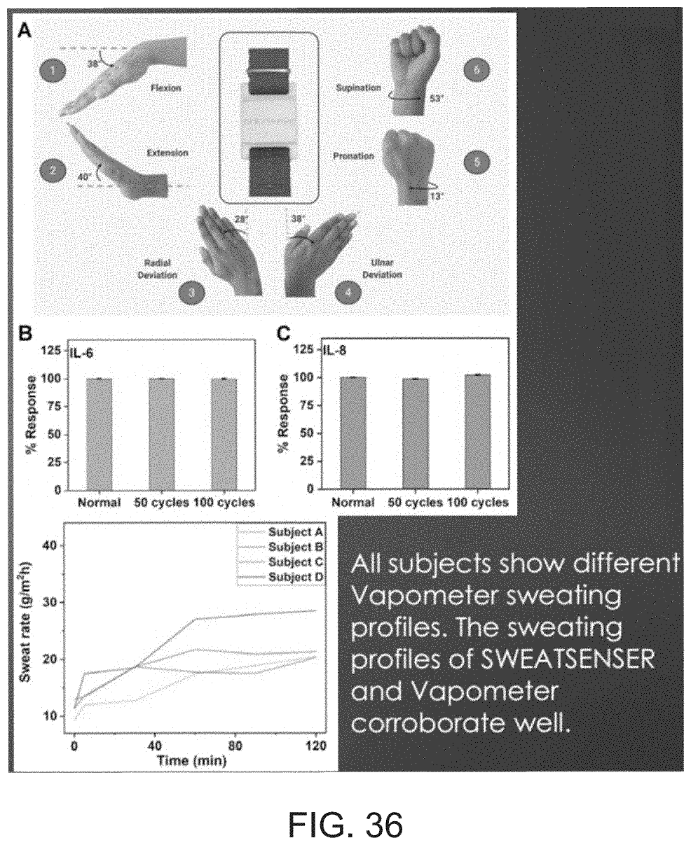

In some implementations, biosensors and biosensor devices (e.g., wearable devices incorporating such sensors) may be included in a system, which includes a health dashboard monitoring system incorporating computer-implemented logic to implement machine-learning and/or predictive analysis methods to report in real-time biomarker levels in biofluids analyzed using affinity-based biosensors devices. FIGS. 35-36 show examples of a biosensor device, which may be utilized with such a health dashboard monitoring system. For instance, FIG. 35A shows an implementation of a wearable armband sensor 3505, incorporating an affinity-based sensor for collecting and generating signals for one or an array of different biomarkers from a sweat sample. While glucose has been named in many of the examples herein, it should be appreciated that one or multiple different biomarkers may be detected in a sample using such a sensor device (and concentrations of the same may be determined using the machine learning models described herein), including (but not limited to) IL6 (pg/mL), IL8 (pg/mL), IL10 (pg/mL), TNFa (pg/mL), IP10 (pg/mL), TRAIL (pg/mL), IL1b (pg/mL), glucose (mg/dL), cortisol (ng/mL), CRP (pg/mL), CALPROTECTIN (ng/mL), galactose (mg/dL), and lactate (mg/dL).

FIG. 35B shows another example implementation of a device 3510 incorporating an affinity-based sensor element (e.g., 3520) that is to collect a sweat sample from the skin 3525 of a subject and generate signals based on the binding of specific, selected biomarkers in the sweat to the sensor element 3520. The device 3510, in this example, may be adhered to the skin of a subject at potentially any suitable or convenient location. Further, the device 3510 may couple to and communicate wirelessly with a personal computing device 3515, which may host software to process data generated by the sensor device 3510 (e.g., to present results generated from a trained and deployed machine learning model present on the sensor device 3510 or the computing device 3515) and deliver additional information to a user (e.g., patient or clinician) relating to the health of a patient based on the determined biomarker concentrations determined through the system.

FIG. 35C is a flow diagram illustrating the example use of biomarker concentration information in connection with delivering health services to a user. For instance, a patient may, through the professional care of a clinician, be diagnosed 3530 with a condition and the monitoring of the progress of that condition may be based on the concentration of one or a collection of biomarkers capable of being measured using a machine-learning-based affinity sensor system (such as described herein). A therapy may be prescribed 3535 in connection with use of the sensor system to monitor biomarker levels in the patient. The results of these biomarker readings may be shared with a software application or service that compares these readings against biomarker levels associated with a wellness level tuned to the specific patient, their diagnosis, and their treatment. For instance, baseline levels may be monitored 3540, biomarkers of the patient may be continuously monitored 3545, immune responses evidenced by these biomarker levels may be analyzed 3550 to determine the progress or changes in illness state (e.g., 3555, 3560) of the patient, to potentially trigger changes or optimizations 3565 to the patient's treatment plan (e.g., by a clinician).

Blood or other body-based biofluids testing methods for detecting biomarkers affecting the quality of life have been prevalent since the innovation of such testing methods and their applications in clinical and non-clinical uses. However, to create a user customized health dashboard using biosensing platforms would require inputs from the user such as calorie intake, exercise, consumption etc. that are measured using sensors on wearable devices for monitoring levels such as sweating rate of the user, body temperature, heart rate, blood oxygen levels and their temporal characteristics, etc. along with the biomarker levels measured in biofluids in real-time. While commercial digital sensors exist for some of these monitors, there isn't one for monitoring and reporting biomarker levels measured in biofluids and specifically sweat, exhaled breath, saliva, etc. in real-time and in a continuous manner. In some implementations, this gap may be overcome utilizing biosensing platform such as discussed herein (e.g., utilizing sweat as a biofluid).

Information obtained from wearable and handheld devices can be converted to meaningful information for suggestive guidance to make informed decisions by the user. A method of conversion of impedance measurements on affinity-based biosensors such as an example sweat-based biosensing platform (e.g., incorporating the features discussed herein) towards reporting biomarker measurements in real-time would bridge the gap for the much-needed information to be generated for the user to make these informed decisions. Such a multi-variable problem can be solved using predictive methods for time-series based analysis. Various models such as auto-regression and neural network-based approaches have been used, as they consider the previous input, the previous output and the current input of the system. Also, these models have been created using interstitial fluid (ISF)-based continuous glucose monitoring systems (CGM), hence these are not based on non-invasively sampled datasets.

Real-time predictive analysis from the outputs of the sensors have been dealt with using either one of or a combination of regression methods, machine learning methods and/or a combination of ordinary differential equations (ODE) and partial differential equations (PDE). However, with an increase in the number of variables, the time and space complexity of the algorithm also increases, resulting in a higher cost of computation. Simplification of ODEs and PDEs has been done to achieve a certain level of linearity at a given operating point using methods such as input-output linearization, however, they come at the cost of customized analysis for a specific use case, adding more complexity to proposing a solution.

In one example, passively expressed eccrine sweat contains a vast array of health information in the metabolites included in eccrine sweat. Proteomic and metabolomic technologies now enable sweat analysis with unprecedented sensitivity and numbers of detected metabolites at the same time (e.g., more than 800 unique proteins and 32,000 endogenous peptides in sweat and opened an exciting field of potential novel, noninvasive biomarkers).

In one example, sweat is collected passively using sweat patches or is actively induced by sweat-sampling devices, such as those discussed herein. In some implementations, active induction is carried out by the topical application of a sweat gland-stimulating substance as well as local current. Despite being noninvasive, current standard sweat sampling remains a challenge, as sample volumes are mostly small. Exercise to induce acute sweating and collect larger amounts of sweat is a potential approach to achieve larger sample volumes but is mostly restricted to healthy subjects. Active induction by external stimulation using local current or by exercise can distort (local elevation or suppression) the target biomarker species concentrations relative to the systemic levels reflective of the disease states in the subject. Hence monitoring in passively expressed eccrine sweat is the only method for establishing a clinically relevant correlation of the disease biomarkers present in circulation which is the current gap and unmet clinical need in accomplishing sweat based disease diagnostics.

The device platform offers real-time, continuous reporting from passively expressed eccrine sweat (1-5 microliters) with no external stimulation and can rapidly detect 3 minutes) and continuously track multiple sweat biomarker levels in a multiplexed manner in a person towards establishing the flare-up and monitoring the progress of illness states in sick subjects. The device platform is based on an electrochemical bio-sensing system that offers real-time, continuous reporting from passively expressed eccrine sweat with no external stimulation. The platform consists of: (i) a disposable and replaceable SWEATSENSER strip that is configured to detect multiple analytes simultaneously from sweat in real-time when worn by the patient; (ii) a wearable Reader onto which the SWEATSENSER strips are mounted and that transduces the outputs of the SWEATSENSER into data consumable by a software application (e.g., integrated into the wearable reader or remote from reader (e.g., transmitted wirelessly from the reader to the computing device (e.g., a smartphone) hosting the application; and (iii) a smart device application that will report the output of the measured cytokines from the patient that the SWEATSENSER strips were configured to detect as plots over time to the wearer/caregiver for information and interpretation. This approach, being 100% non-invasive (e.g., no needles, no punctures, no pain), has no known anticipated medical risks or safety issues to the wearer.

In another example, a handheld biosensor may also be provided to utilize the sensors above. For instance, a handheld READ platform, akin to that of a blood glucometer-like device, can be configured to detect and report multiple biomarkers, all from a single test of the patient's sample specimen (e.g., sweat, saliva, blood, plasma, nasopharyngeal, urine, etc.). The platform may include:
1. A disposable, single-use sensor cartridge with an array of sensing electrodes that are individually configured, and surface functionalized with biomarker specific capture probes to detect multiple biomarkers simultaneously from saliva in real-time (e.g., such as in the sensors solutions discussed above);
2. A handheld palm-sized or smaller form-factor electronic reader onto which the sensors are mounted that transduces the electrical outputs resulting from affinity binding to target biomarkers in saliva to other electronic devices/data servers through an app interface (e.g., configurable to support both wired or wireless communication);
3. A smart app (e.g., configurable to work with and available for Windows/Android/IOS platforms) that will report the output in real-time of the measured biomarkers to the clinician for interpretation and decision making.

The affinity biosensor on which both the wearable and handheld biosensing platforms are based on is designed to monitor the binding of the target analytes and uses specific binding of antibodies, or antibody-related substances, enzymes, peptides, and nucleic acids for biomolecular recognition. The target analytes interact with the sensing electrode functionalized through selective surface treatments applied to the surface for the specific target detection. Electrode stability and immobilization efficiency of biomolecules onto the sensing surface are critical for highly accurate performance (stable and consistent signal-over-noise) of biosensors. The impedance Z of the sensor is determined by applying a voltage perturbation with a small amplitude and detecting the output current response for the specific target detection. The measured impedance associated with target biomolecule binding is a complex value, since the current can differ in terms of not only the amplitude but also it can show a phase shift ϕ compared to the voltage-time function. Therefore, the results of an impedance measurement illustrated using a Bode plot, which plots log |Z| and ϕ as a function of log f, and using a Nyquist plot, which plots Z_{Real} and Z_{imaginary}, are calibrated to report the concentration values of the target in the saliva specimen. All this preprocessing is performed in the electronic reader onto which the sensors are mounted and outputs the impedance measurements over time that is specific to the biomarker levels being sampled within the biofluids.

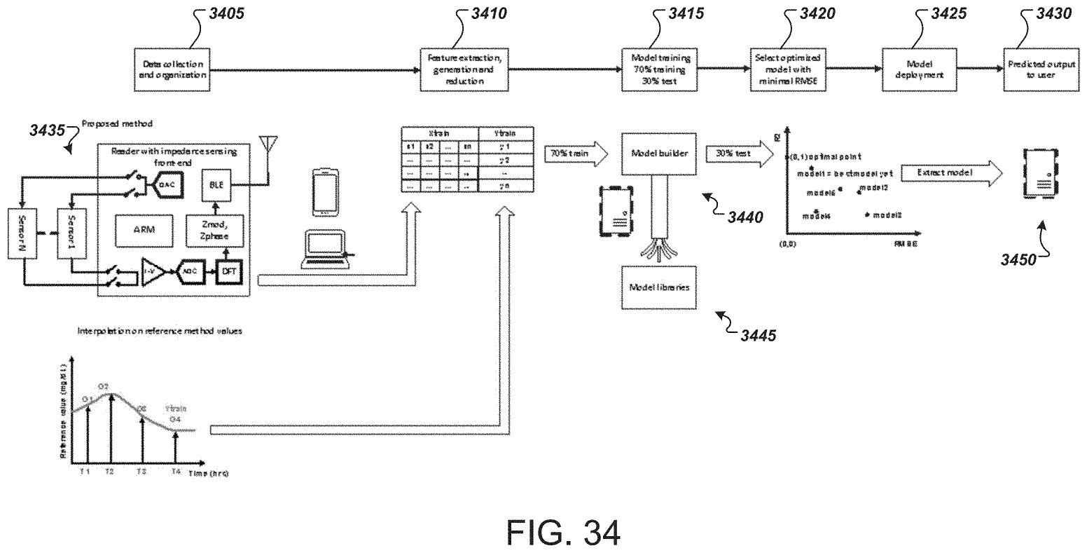

FIG. 34 is a block diagram illustrating aspects of an example machine-learning based biosensor system. For instance, development and training of a proposed machine-learning model for use in connection with a biosensor system may include data collection and organization (3405), with data being collected and correlated from both the affinity-based sensor 3435 (e.g., a time series of readings characterizing the impedance generated at the sensor based on the binding of biomarker material to the sensor's receptors when a test sample (e.g., sweat) is brought into contact with the sensor), supplemental biosensors (e.g., characterizing corresponding biometric characteristics), and verification sensors (e.g., a blood glucose meter for verifying the concentration of glucose in an implementation where glucose is being measured) already tuned to accurately assess the concentration of the biomarker and serve as the ground truth used during supervised training of the machine learning model. Feature extract, generation, and reduction (3410) may be performed and the results used to perform model training (3415) (e.g., 70/30 train/test for example). In some implementations, multiple alternative models may be trained and tested (e.g., by a model builder tool 3440) for an affinity-based sensor's measurement of a corresponding biomarker. The best performing one of the models 3450 (e.g., selected from a model library 3445) may be selected (3420) (e.g., based on the model tested to perform with minimal RMSE (Root Mean Square Error)) and deployed in connection with the affinity-based sensor (3425). The readings generated by the affinity-based sensor may then be provided to the trained model to determine a predicted biomarker concentration output 3530. While some implementations may train the machine learning model to generate predictions from data describing the electrical characteristics of the signals generated by the affinity-based sensor, other implementations may be trained to generate the prediction (e.g., predicted biomarker concentration) from an array of data including both data describing characteristics of the electrical signal generated by the affinity-based sensor as well as other biometric sensors (e.g., describing biometric information of the subject/user).

In some implementations, training may be carried out on a continuing basis. For instance, following initial training and deployment of a machine learning model for an affinity-based sensor, the sensor may be used by a user and continuously trained (e.g., using data from an extraneous sensor for supervised learning) to further improve performance of the machine learning-based affinity sensor system and tune the training to the particular biology of the patient.

The unprocessed data collection and organization are handled at the primary point of data generation, such as a handheld or wearable electronic reader module that is connected to the sensor strips with an analog front end and wired or wireless capability. For a preset sampling frequency, a time series is generated for each sensor channel with time, Z_{mod}, and Zₚₕₐₛₑ and stored on local memory of the electronic reader. These time series are extracted at the end of data collection step or at an on-demand basis and brought to a model builder system that is part of or resides on a smartphone system using an app interface. The model builder may also be a part of or reside on a cloud server or a local computer.

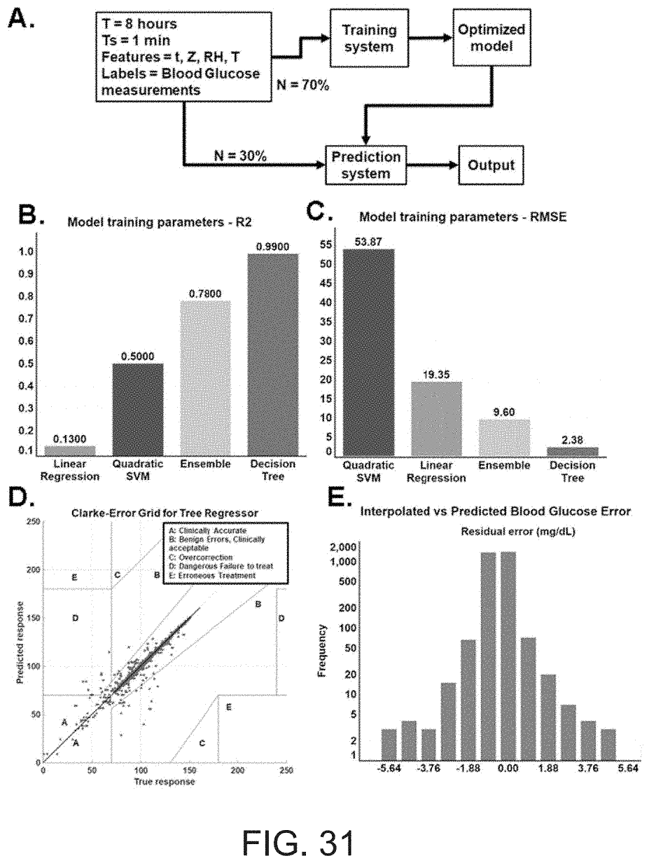

Along with this process of data collected from the biosensing platform, static temporal measurements of the biomarkers of interest are taken in the same body fluids or different body fluids for which a reference method has been pre-established i.e. for example using finger-pricked blood glucometer as the reference method for calibrating the sweat glucose impedance measurements from the biosensor. These reference measurements taken at static time points over the period of testing are extended to a time series curve to map with that of the impedance measurements collected from the electronic reader over the whole testing period via a suitable interpolation technique. These are considered as Yₜᵣₐᵢₙ, whereas the impedance data collected from the electronic reader is considered Xₜᵣₐᵢₙ. Additional features in Xₜᵣₐᵢₙ columns may be created, i.e. running difference of Zₓ, percent change of Zₓ, human subject temperature, skin humidity, caloric intake and demographic information to add as much information to the time series as possible to facilitate a detailed overview of the training data. The interpolation process is shown in FIG. 31 for reference.

The model builder takes in the train split of the data and runs multiple models based on the availability of model libraries. The rest test split data is used to predict and compare the data against the reference method. For a certain number of models that may be created, the error of the prediction with respect to the reference method may be generated using a Root Mean Square Error (RMSE) function. Moreover, the goodness of fit may be established using the R2 (correlation spearman's or pearson's or equivalent used for statistical matching) value. To obtain the best RMSE and R2 combination, the outcome may be plotted in Euclidian space to check their Euclidian distance from the optimal (0,1) point. The model closest to the optimal point may be extracted, deployed and used for predicting concentration of biomarkers in subsequently collected data.

In FIG. 31, diagram (A) below shows the machine learning architectures of the proposed training system for predicting the blood glucose concentrations from the glucose biomarker levels measured in sweat using the affinity based calibrated impedance response from the sweat sensor device. The training was performed on N=20 time series by splitting the collected data into groups of train time series and test time series in a 70:30 splits. The features used were time elapsed, impedance, perspiration (RH) and skin temperature, whereas the labels were discrete blood glucose values from a glucometer converted to a continuous time series using bicubic interpolation. Various commonly known training models such as linear regression, quadratic support vector machine (SVM), bagged ensemble regression and decision tree regression were studied for this application. The selected algorithm for the target biomarker being measured in sweat can be chosen for lowest RMSE/MSE (root mean square error/mean square error) and highest R2 (correlation between measured and prediction). Diagram (B) shows the highest R2 for the decision tree regression system. This is corroborated by the lowest root mean square error of 2.38 mg/dL for the decision tree regression as shown in Diagram (C). The predicted response for the test dataset was further validated to a clinical standard using a Clarke error grid as shown in Diagram (D) where majority of the predicted response is found to be in region A. This proves the clinical feasibility of the predictor system against a known reference method. Diagram (E) is the residual error histogram which was obtained by the difference of the true and the predicted response. The mean of the normal distribution of error is centered close to 0 mg/dL with a span of +/−6 mg/dL.

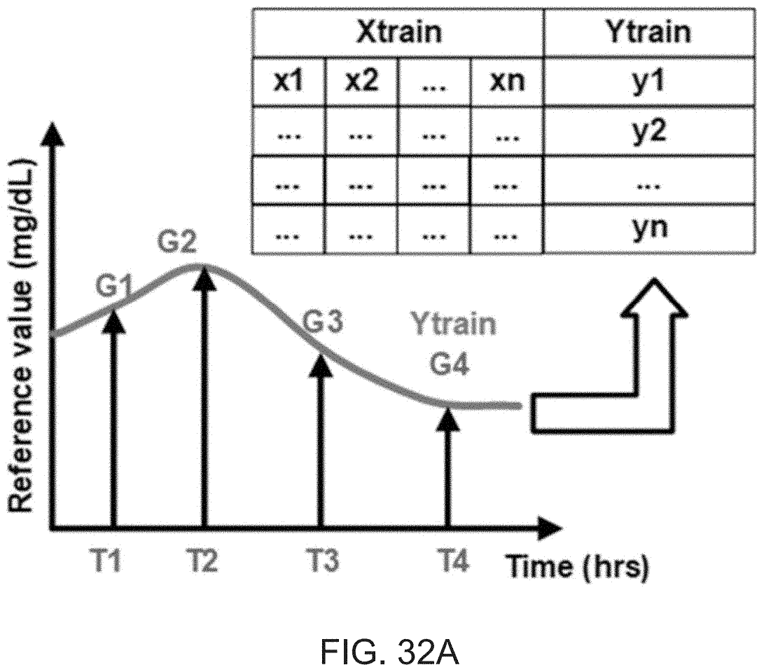

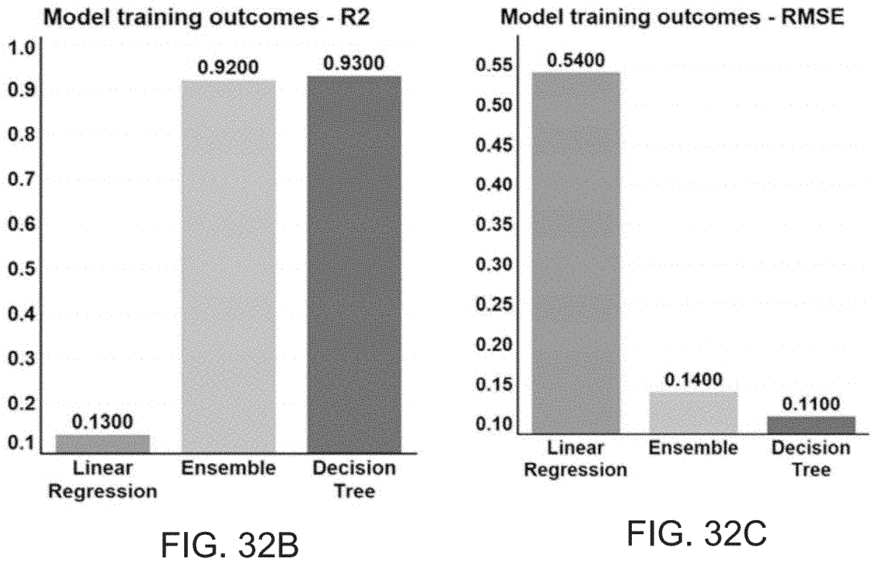

In another example, represented by the block diagram shown in FIG. 32A, a model can be built for continuous signal and the conversion of the measured input parameters to glucose concentrations using discrete datapoints collected from an affinity-based sensor. The glucose concentrations from the sweat collected at discrete timepoint (e.g., and measured using ELISA method) are used to interpolate with the impedance signal matching with those time points to obtain a smooth and continuous time based sweat glucose concentration output from the continuous time-based impedance signal of the wearer. Given the varying nature of the glucose molecule over time we use bicubic method of interpolation. The obtained continuous signal is used as the output parameter for regression building. The continuous signal is obtained for one minute of frequency. This interpolation methodology allows to perform on-demand sampling of the glucose with very good accuracy.

Various regression techniques may be utilized to train a model for prediction of biomarker concentration using an affinity-based sensor, such as described, including techniques such as linear regression, decision tree regression, and ensemble regression algorithms. The graphs of FIGS. 32B-32C show that, in one example, the relative performance of different algorithms. In one example, the ensemble and decision tree regression algorithms performed the best. For instance, the model selection (such as in this example) may be based on success measure criteria such effective R2 value of the model and root mean square error (RMSE) value. The objective here is to achieve RMSE of +/−20% of the expected sweat glucose value. And R2 value greater than 0.8. The results are plotted as the bar graph as seen in the G. 32B. The plotted values are the cross validation mean values for k=10. For simple linear regression the R2 value of 0.12 and RMSE of 0.54 is achieved in the example of FIGS. 32B-32C, both these values fail to satisfy the objective criteria for the model. For the decision tree model and ensemble model similar R2 of 0.93 and 0.94 are observed in this example, meeting the R2 objective criteria. Both the decision tree and ensemble showed comparable RMSE values of 0.1 and 0.15 and hence either would be a good fit for RMSE objective in this example based on this example criteria.

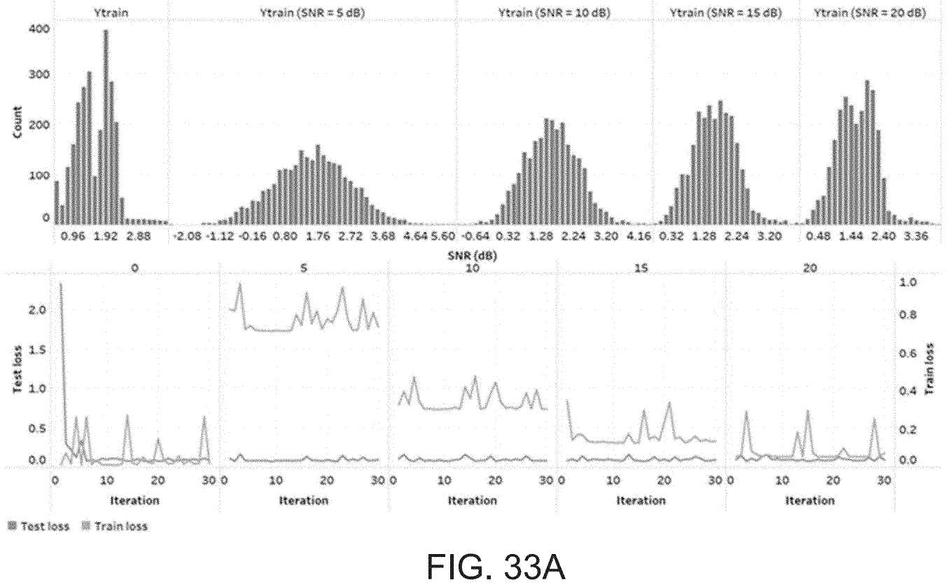

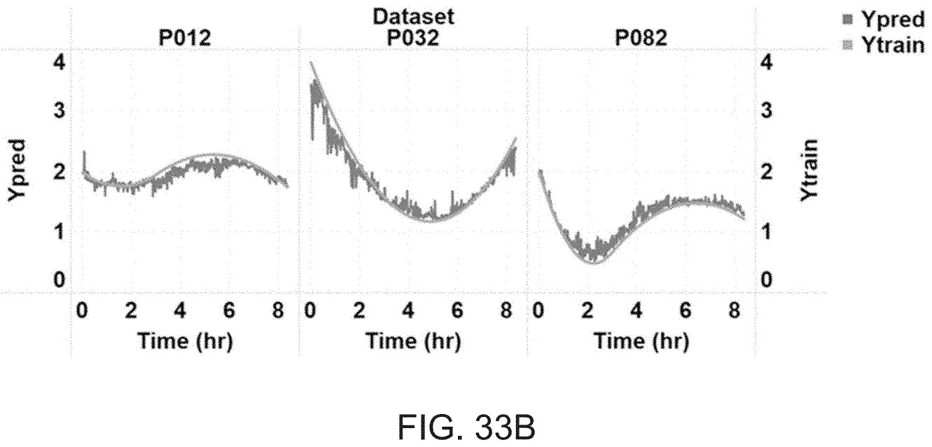

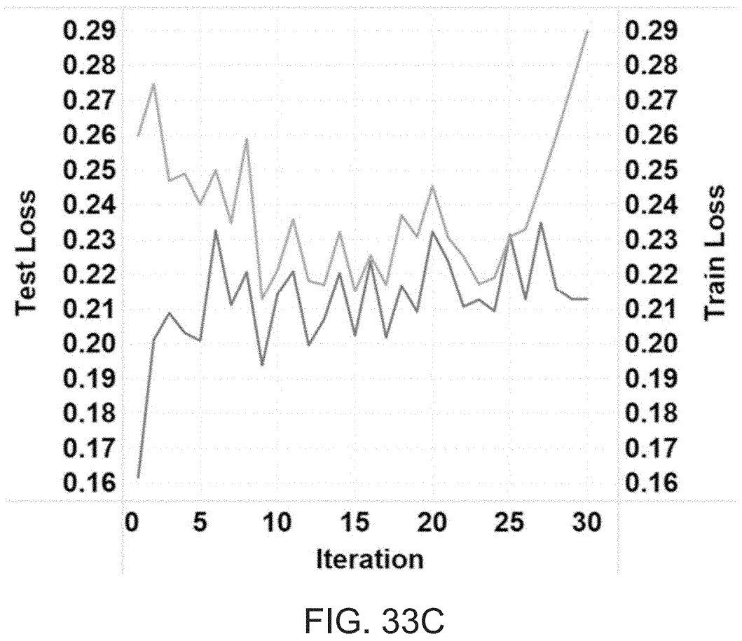

FIG. 33A is a graph showing an example noise addition feature that may be applied during training of a machine learning model (used to predict biomarker concentration from electrical characteristics of a sensor signal) to introduce the variability for generalization. The results obtained from the interpolation are vulnerable to real-world noise from various sources. To address these shortcomings, a Gaussian noise parameter, also known as additive white noise, may be introduced to the results obtained from the interpolation in some implementations. The results obtained after adding white noise resulting in response signals with signal-to-noise (SNR) ratios of 1, 5, 10, 15, and 20 dB were analyzed to establish the optimal levels to use in the model. The objective is to minimize the loss but also to allow room for generalization and avoid overfitting. From FIG. 33A, an SNR of 10 dB met this requirement of balancing the train and test loss with the minimum gap between the predicted and actual output. In the case of higher SNR values, the train and test loss look very similar to the response signal without any noise. In the case of lower SNRs, the train and test loss values do not seem to converge, showing an error of >20%, which in some applications is beyond the acceptable clinical limits. For instance, an SNR of 10 dB may be determined to be the optimal SNR ratio for the generalization of the model training process.

FIG. 33B is a graph showing a set of results obtained from testing example algorithms for sweat glucose reporting on human subjects. The graph of FIG. 33B shows the algorithm test on three subjects. The predicted value follows the trends for the sweat glucose value. The sweat values for the test subjects are converted to continuous curves using the same bicubic interpolation methodology used for building the continuous monitoring set values. The predicted values show the presence of the noise. Decision tree model building has used two types of generalization techniques. In one example, L1 regularization offered by the algorithm is used to reduce the statistical overfit of the model. Additionally, external white noise is added to the level of the SNR=10 dB signal to the training values. The noise addition takes care of variability that might be present in the actual signal. The overall results give a good fit when the predicted signal is compared with the real movement.

As the measure to prevent overfitting, the error vs. epoch graph obtained on the training dataset was overlaid with the utterly unknown dataset used as the test and is plotted in the example graph shown in FIG. 33C. The loss is plotted on the y-axis, and the x-axis is the epoch used for the training. The objective here is to minimize the loss but also to avoid overfitting. As seen in the graph as the initial training epochs, high bias behavior was observed. A more significant difference between the training error and test error indicates the need for more training. As the number of epochs increases, the training loss and test loss both show a declining nature. At epoch 24, the minimum difference between the training error and test error is achieved. Additionally, the training error is constant at the same point, establishing a balance between loss minimization and overfitting risk.

More generally, training by interpolation is a method of “up-sampling” a time series dataset using a reference method where multiple time points cannot be collected due to practical implications of creating training data. In this blood glucose validation example, the reference method, i.e. the finger-prick glucometer measurements cannot be taken more than 4-5 times a day due to inconvenience caused to the human subject. The EnLiSense reader can take more rapid samples, i.e. one sample per minute measurements. Hence, for an 8-hour test period, the reader yields 480 samples, whereas the glucometer yields 4 samples. Due to such a huge difference in the number of Xtrain and Ytrain samples, one can use interpolation to fit a curve on lesser Ytrain points. This will yield an interpolated 480-point Ytrain series per subject, which can be used to perform a point-to-point regression on the time series data.
1. Use of electrical signals from affinity-based biosensors to measure the biomarker levels from biofluids on human subjects for correlating and reporting the equivalent biomarker levels in other biofluids for which a reference method may be already established. For example, correlation of sweat biomarker levels to the equivalent blood biomarker levels.
2. Generation of additional features for training data with respect to the behavior of an affinity-based sensor such as running difference of Zₓ, percent change, demographic information of human user
3. Use of interpolation technique on low-sampling rate reference methods to match sampling rate of reader proposed in 1 and the use of Euclidian coordinates to find optimal points using Euclidian distance of model.
4. Use of the up-sampled time-series in (2) as labels and inputs such as electrical biosensor signal, body temperature, skin humidity, demographic information, etc. as in (1) as features to train a machine-learning regressor and/or classifier. The machine-learning model may be one of the following but not inclusive of any feature-label combination such as point-to-point, series-to-point, series-to-series, matrix-to-point or matrix-to-series classifier and/or regression.
5. Integration of a predictive machine-learning model as per (3) to predict biomarker values, inclusive of but not limited to, in wearable or handheld electronic reader firmware, smartphone application, computer software or cloud-based computation hardware.
6. Implementation of a machine-learning model as per (3), (4) to predict and retrieve biomarker values from a sweat-biosensor enabled reader in-situ, and/or via wired/wireless communication on a smart device, computer, webpage or data repository (including but not limited to localized, centralized or decentralized databases and version-controlled data repositories).

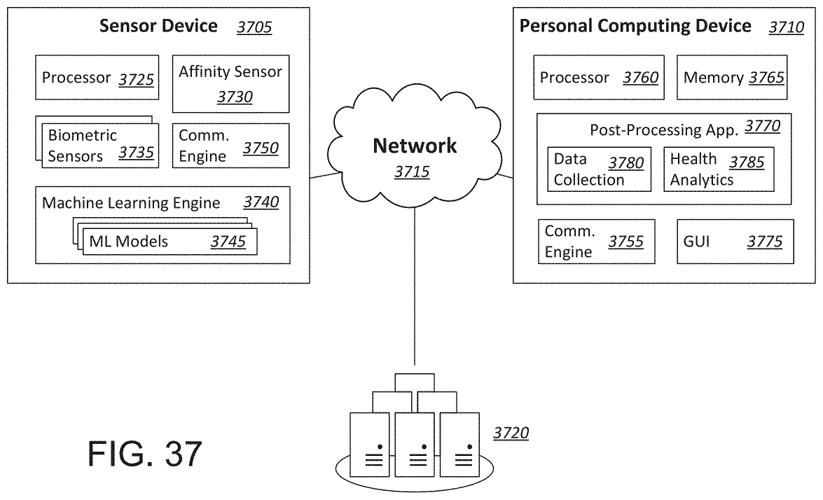

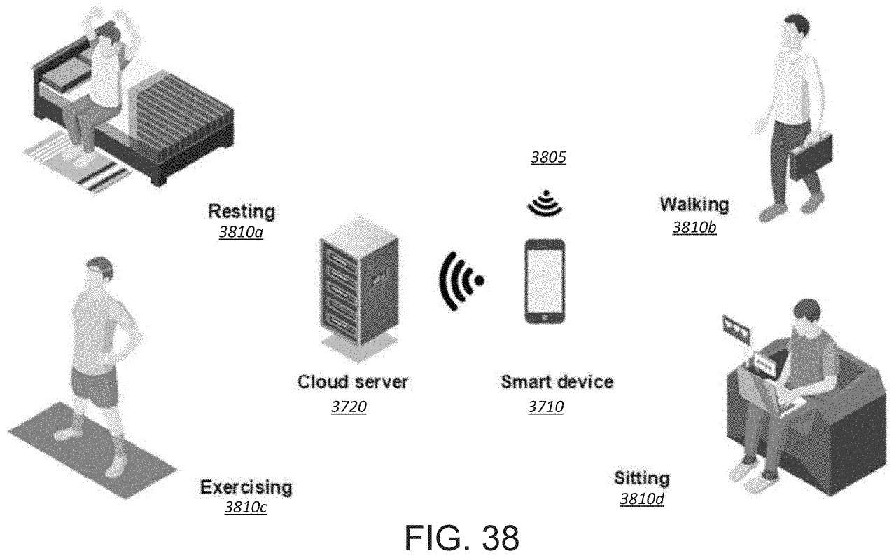

FIG. 37 is a simplified block diagram illustrating an example sensor device 3705, which may, in some implementations, be embodied as a READ device, SWEATSENSER reader, or other wearable sensor device. The sensor device 3705 may be utilized together with a personal computing device 3710, such as a smartphone, smartwatch, laptop, desktop, or other personal computing device. The sensor device 2705 and personal computing device 3710 may communicate over one or more networks, such as wireless networks utilizing WiFi or Bluetooth. A cloud-based service (e.g., hosted in a distributed computing or cloud computing system (e.g., 3720) may likewise be utilized and the personal computing device and/or sensor device may communicatively couple to this cloud system and share data for further storage or analytics.

In one implementation, the sensor device 3705 may include a processor 3725 capable of executing logic and directing operation of the logic of the sensor device. In one example, the sensor device 3705 may possess both affinity-based sensors 3730 (such as discussed above, which are adapted to bind to specific biomarkers and generate electrical signals based on this binding), as well as more generalized biometric sensors 3735 (e.g., to detect temperature, skin moisture level, pulse, blood oxygen content, movement (e.g., steps), among other biometric readings). Arrays, vectors, matrices, or other data sets may be generated at the sensor device to incorporate readings from the affinity sensor(s) 3730 and biometric sensor(s) 3735. Such data sets may capture contemporaneous readings of these combined sensors 3730, 3735 and may be provided as an input to a machine learning engine 3740 for further processing. In some implementations, the readings of the affinity sensor may be electrical characteristics of the electrical signal generated at the sensor 3730 based on the binding of a particular biomarker to the analytes or binding substance utilized in the sensor 3730. The individual electrical characteristics, such as voltage, amperage, impedance, impedance phase shift, etc., may each be provided as a particular data point within the data set, along with particular data points representing biometric values sensed by biometric sensor(s) 3735, among other examples.

The machine learning engine 3740 may provide the data set input to one or more machine learning models (e.g., 3745). A machine learning model 3745 may be trained to determine, from the data set, an amount of the biomarker detected by the affinity-based sensor (e.g., based on the combination of electrical characteristics expressed by the affinity sensor in response to detecting this amount of the biomarker in a body fluid sample (e.g., sweat, saliva, urine, blood, etc.). Different machine learning models 3745 may be provided which have been trained to predict amounts of a biomarker. In instances where the affinity sensor comprises an array of sensors (e.g., each with a unique binding to bind to a respective biomarker), the multiple machine learning models may include respective models trained for determining an amount of a corresponding one of the multiple biomarkers capable of being detected by the affinity sensor, among other example embodiments.

As discussed herein, the sensor device 3705 may continually collect body fluids and generate corresponding signals using the affinity sensor 3730 based on the binding of particular biomarkers present in the body fluid. For instance, a user wearing the sensor device 3705 may sweat in different amounts throughout the day and the affinity sensor may detect and respond to the presence of specific biomarkers present in the wearer's sweat. Further, biometric readings may be captured contemporaneously with the generation of signals by the affinity sensor to identify biometric conditions of the wearer that may relate to how the user is sweating (e.g., body temperature, activity level, heart rate, etc.). Accordingly, the sensor device may continuously generate data sets from the affinity sensor and biometric sensor data to deliver as inputs to the machine learning engine to generate, from machine learning models, predicted values for the amount of a specific biomarker contained in the sweat of the wearer.

Continuing with the example of FIG. 37, the sensor device may share the predicted biomarker amounts determined using the machine learning engine 3740 with a personal computing device. A communication engine 3750 of the sensor device may pair with the personal computing device and/or encrypt or otherwise protect the data generated by the machine learning engine for secure and private sharing of this data with the personal computing device. Likewise, the personal computing device 3710 may possess a communication engine 3755 to effectively receive data from and communicate with the sensor device 3705. The personal computing device may host one or more software applications (e.g., 3770), stored in memory 3765 and executed by a processor 3760 of the personal computing device. Such applications may include health monitoring, clinical, or other health-related applications, which may advantageously use, as an input, a stream of real time readings from the sensor device 3705 identifying biomarker and other biometric information of the sensor device's wearer. In some implementations, the application 3770 may perform post-processing on the data generated by the sensor device, including biomarker amount data generated by the machine learning engine 3740 based on readings of the affinity sensor 3730. The application 3770 may include data collection logic to aggregate data from the sensor device 3705 (and potentially other sources), as well as to organize data received from the sensor device 3705 (e.g., data received in multiple communications over a period of time) to generate time series data describing biometric readings and trends, based on readings from the sensor device 3705, among other examples. The application 3770 may additionally possess health analytics logic to utilize this data to generate recommendations, alerts, displays, metrics, and other information for display to a user (e.g., the wearer of the sensor device, a care giver, or medical service provider). Such information may be presented to the user via a graphical user interface 3775 (or audio interface, tactile interface, etc.) of the computing device 3710. In some implementations, the application 3770 may make use of data or services from one or more cloud services (e.g., hosted in a cloud-based system 3720) to generate enhanced results and services at the personal computing device in association with a user's use of sensor device 3705, among other example uses and embodiments.

FIG. 38 is a block diagram illustrating example use of a sensor device within such a system. For instance, a sensor device may generate results 3805 (e.g., using machine learning analysis of an affinity-based sensor's readings) indicating amounts of one or more biomarker(s) detected in body fluid (e.g., sweat) of a user, which are communicated to a cooperating smart device 3710. The wearable sensor device may be worn by the user throughout the day and during various activities 3810a-d performed by the user.

Diabetes is one of the most common chronic diseases that occurs due to an imbalance in the glucose levels of the body. Continuous monitoring of the glucose level of a patient is critical to managing this disease. Existing glucose monitors (e.g., utilizing blood samples from finger pricks) continue to pose a hurdle to patients' regular and reliable use of such monitors. The sensor device solutions discussed above may be utilized to implement a non-invasive continuous glucose monitoring system based on sweat glucose collected as a body fluid sample and using the machine learning approaches discussed herein. Sweat glucose shows good correlation with blood glucose currently relied upon in managing diabetes and pre-diabetes. Accordingly, reliable, non-invasive measurement of sweat glucose would lead to better healthy lifestyle using such a non-invasive continuous monitoring system. As discussed above, an affinity-based sensor may be an electrochemical sensor, which gives impedance-based responses. These impedance-based responses (describing an electrical signal generated by the affinity-based sensor), may be processed further using machine learning to calculate the sweat glucose concentration. Data pre-processing may also be performed to take care of any missing values or null values that might materialize in the real time data stream (e.g., from the affinity-based sensor readings or the supplemental biometric sensor readings). For example, if the contemporaneous temperature or skin moisture value is missing then these may be replaced by interpolation or by reusing a previously calculated average value, among other example techniques. For instance, a reading of the electrical signal generated at the affinity-based sensor (configured to bind to sweat glucose) may be captured according to a certain interval (e.g., every minute), and corresponding readings of the biometric sensors may also be captured.

Such a sweat sensing platform implemented using the features and solutions discussed herein, utilizes an electrochemical bio-sensing system that offers real-time, continuous reporting from passively expressed eccrine sweat. Some implementations may rapidly detect and continuously track multiple biomarker levels in a multiplexed manner. In one example, the platform may utilize a disposable and replaceable sensor strip and a wearable reader onto which the sensor strips are mounted and that transduces the outputs wirelessly to the data server through a coordinating software application.

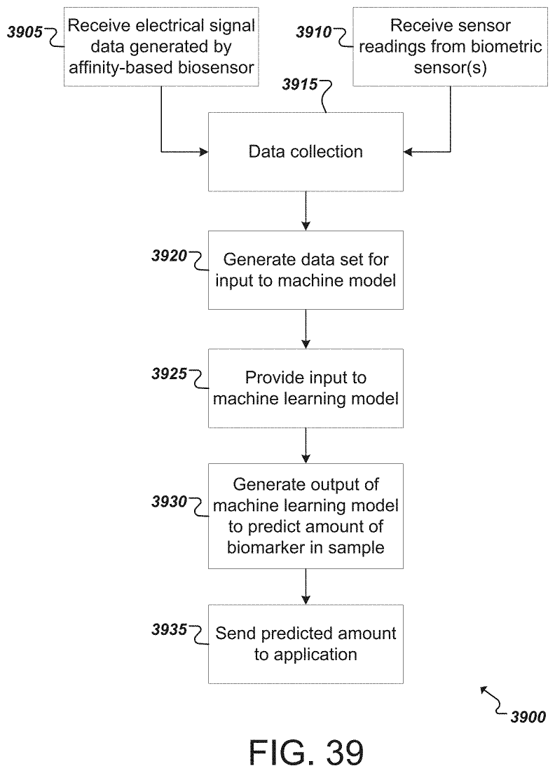

Turning to FIG. 39, a simplified flow diagram 3900 is shown illustrating example processing of sensor data generated by an example sensor device, such as described in the implementations above. For instance, a sensor device may include affinity-based sensors and non-affinity-based biometric sensors. The affinity-based sensors may generate electrical signals based on the binding of targeted biomarkers, but determining the specific amount of the biomarkers from a sample may be challenging from the electrical signals alone. The affinity-based sensor (or post processing circuitry) may identify 3905 electrical characteristics of the electrical signal, such as characteristics of an impedance measured for the affinity-based sensor's signal. Additional information may also be collected contemporaneously with the information about the affinity-based sensor's signal, including biometric information received 3910 from the non-affinity-based biometric sensors. This data may be correlated and collected (at 3915) to generate a data set 3920 for use as an input to be provided 3925 to a machine learning model trained to predict an amount of the biometric detected by the affinity-based sensor from the combined data points included in the data set 3920. As examples, such trained machine learning models may include decision tree regression models, ensemble regression models, neural networks, and other example machine learning models. Such machine learning models, in some implementations, may be trained in a supervised manner (e.g., using other high-precision biomarker sensors to provide a ground truth value for use in training (e.g., glucose reader results to train a machine learning model for use in detecting an amount of glucose detected by a corresponding affinity-based sensor based on characteristics of an electrical signal generated by the affinity-based sensor from sweat containing the amount of glucose), etc. The machine learning model may generate an output 3930 to represent the determined amount of the biomarker, and in some instances, this output may be shared 3935 with an application (e.g., hosted on the sensor device or a cooperating computing device (e.g., a smart phone paired to the sensor device), for use by the application in providing further services relating to the monitoring of this biomarker, among other example uses.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. At least one non-transitory machine-readable storage medium with instructions stored thereon, the instructions executable by a machine to cause the machine to:
detect electrical characteristics of an electrical signal generated by an affinity-based senor, wherein the affinity-based sensor is configured to bind to a particular biomarker within a body fluid sample and generate the electrical signal based on binding to the particular biomarker;
detect one or more biometric characteristics of a subject from one or more other sensors;
provide, as an input to a machine learning model, a data set comprising data describing each of the electrical characteristics and each of the one or more biometric characteristics; and
generate an output of the machine learning model from the input, wherein the output identifies an amount of the particular biomarker present in the body fluid sample based on the input.

2. The storage medium of claim 1, wherein the affinity-based sensor generates a continuous stream of electrical signals and a respective input is generated for each sensor reading in the continuous stream and provided to the machine learning model to generate a corresponding stream of outputs of the machine learning model.

3. The storage medium of claim 1, wherein the instructions are further executable to cause the machine to transmit the output to another computing device for additional processing and presentation of a reading related to the particular biomarker to a user.

4. The storage medium of claim 1, wherein the body fluid sample comprises human eccrine sweat.

5. The storage medium of claim 4, wherein the particular biomarker comprises glucose.

6. The storage medium of claim 1, wherein the body fluid sample comprises one of human saliva, sweat, urine, or aerosol.

7. The storage medium of claim 1, wherein the body fluid sample comprises a less than ten microliter sample.

8. The storage medium of claim 1, wherein the affinity-based sensor comprises a semiconductive material to which a binding substance is suitably immobilized, wherein the binding substance is to bind to the particular biomarker.

9. The storage medium of claim 1, wherein the machine learning model comprises a decision tree regression model.

10. The storage medium of claim 1, wherein the machine learning model comprises an ensemble regression model.

11. The storage medium of claim 1, wherein the electrical characteristics comprise characteristics of electrical impedance measured at the sensor based on the binding to the particular biomarker.

12. The storage medium of claim 11, wherein the electrical characteristics comprise one or both of phase shift or amplitude of the electrical impedance.

13. The storage medium of claim 1, wherein the biometric characteristics comprise at least one of a temperature of a subject or skin humidity of the subject.

14. The storage medium of claim 1, wherein the biometric characteristics are sensed contemporaneously with capture of the body fluid sample.

15. The storage medium of claim 14, wherein the affinity-based sensor and the one or more other sensors are present on a wearable sensor device.

16. A method comprising:
detecting electrical characteristics of an electrical signal generated by an affinity-based senor, wherein the affinity-based sensor is configured to bind to a particular biomarker within a body fluid sample and generate the electrical signal based on binding to the particular biomarker;
detecting one or more biometric characteristics of a subject from one or more other sensors;
providing, as an input to a machine learning model, a data set comprising data describing each of the electrical characteristics and each of the one or more biometric characteristics; and
generating an output of the machine learning model from the input, wherein the output identifies an amount of the particular biomarker present in the body fluid sample based on the input.

17. A system comprising:
means to detect electrical characteristics of an electrical signal generated by an affinity-based senor, wherein the affinity-based sensor is configured to bind to a particular biomarker within a body fluid sample and generate the electrical signal based on binding to the particular biomarker;
means to detect one or more biometric characteristics of a subject from one or more other sensors;
means to provide, as an input to a machine learning model, a data set comprising data describing each of the electrical characteristics and each of the one or more biometric characteristics; and
means to generate an output of the machine learning model from the input, wherein the output identifies an amount of the particular biomarker present in the body fluid sample based on the input.

18. A system comprising:
a processor;
a sensor device comprising:
an affinity-based sensor to:
generate an electrical signal based on presence of a particular biomarker in a body fluid sample provided to the affinity-based sensor, wherein the affinity-based sensor is configured to bind to the particular biomarker within the body fluid sample and generate the electrical signal based on binding to the particular biomarker; and
detect electrical characteristics of the electrical signal; and
one or more other sensors to detect one or more biometric characteristics of a subject from one or more other sensors;
machine learning engine executable by the processor to:
receive an input to a machine learning model, wherein the input comprises a data set comprising data describing each of the electrical characteristics and each of the one or more biometric characteristics; and
generate an output of the machine learning model from the data set, wherein the output identifies a predicted amount of the particular biomarker present in the body fluid sample.

19. The system of claim 18, further comprising an application to accept the output of the machine learning model and generate result data based on the output for presentation to a user.

20. The system of claim 19, wherein the application is run on a computing device separate from the sensor device and the machine learning engine is executed on the computing device.

21. The system of claim 19, wherein the application is run on a computing device separate from the sensor device and the processor and the machine learning engine are present on the sensor device.

22. The system of claim 21, wherein the sensor device comprises a wearable sensor device.
